SEQUENCE LISTING (2 sequences)

SEQ ID NO: 1 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 9 aa, Artificial Sequence
  Synthetic Peptide
IEVMYPPPY

(19) United States
(12) Patent Application Publication
(10) Pub. No.: US 2021/0379057 A1
(43) Pub. Date: Dec. 9, 2021

(54) NUTLIN-3A FOR USE IN TREATING A MYCOBACTERIUM TUBERCULOSIS INFECTION

(71) Applicant: Massachusetts Institute of Technology, Cambridge, MA (US)

(72) Inventors: Alexander K. Shalek, Cambridge, MA (US); Travis K. Hughes, Cambridge, MA (US)

(21) Appl. No.: 17/285,629

(22) PCT Filed: Oct. 16, 2019
(86) PCT No.: PCT/US2019/056599

§ 371 Date: Apr. 15, 2021

Related U.S. Application Data
(60) Provisional application No. 62/746,516, filed on Oct. 16, 2018.

(51) Int. Cl. A61K 31/496; A61K 35/15; A61P 31/06

(52) U.S. Cl. CPC A61K 31/496; A61P 31/06; A61K 35/15

(57) ABSTRACT

Methods and compositions for treating or preventing a disease by modulating a microenvironment of a cell or cell mass in a subject, the method comprising administrating an effective amount of one or more modulating agents that modulate mast cells, plasma cells, Th1-Th17 cells, and/or CD8+ T cells in the subject.

### CROSS-REFERENCE TO RELATED APPLICATIONS

This application claims the benefit of U.S. Provisional Application No. 62/746,516, filed Oct. 16, 2018. The entire contents of the above-identified application are hereby fully incorporated herein by reference.

### TECHNICAL FIELD

The subject matter disclosed herein is generally directed to methods and compositions for modulating microenvironment of a cell or cell mass.

### REFERENCE TO AN ELECTRONIC SEQUENCE LISTING

The contents of the electronic sequence listing (“BROD-2585WP_ST25.txt”; Size is 1488 bytes; was created on Oct. 16, 2019) is herein incorporated by reference in its entirety.

### BACKGROUND

Macrophages are thought to control Mycobacterium tuberculosis (Mtb) infection but have only modest capacity to kill the bacterium. Mechanistically, Mtb survival inside macrophages has been attributed to its ability to disrupt phagolysosomal maturation as well scavenge nutrients from the host. However, it has recently been appreciated that the limited bacterial control exerted by a population of human macrophages may reflect the aggregate effects of simultaneous bacterial clearance and growth. How and where this variation emerges during the interaction between bacterium and pathogen, and whether there are features that distinguish macrophages that successfully clear Mtb from those that do not, remains poorly understood.

Differences between macrophages have historically been understood in terms of polarization state; however, several recent studies point to a much more complex model integrating differentiation, activation, and tissue cues as signals that can influence macrophage state2. However, these paradigms, which have largely been understood using bulk approaches, do not explain the emergent heterogeneity in macrophage functional state that is apparent during Mtb infection. Recently, single-cell transcriptional profiling and dynamic imaging of myeloid cells has revealed surprising cell-to-cell variability in basal transcription and the responses evoked by purified toll-like receptor (TLR) stimuli despite homogeneous culture conditions3-5. It is unknown whether these variations are propagated into durable differences in macrophage fate or functional capacity.

### SUMMARY

In one aspect, the present disclosure includes methods for treatment or prophylaxis of a Mycobacterium tuberculosis (MTB) infection comprising modulating expression of one or more genes in a mast cell, a plasmablast, or a combination thereof, in a subject in need thereof, wherein the one or more genes expresses at a level different in a resolving granuloma in an MTB infected tissue comparing to a level in a progressing granuloma in the MTB infected tissue.

In some embodiments, the modulating comprises upregulating the expression of the one or more genes, wherein the one or more genes expresses at a higher level in a resolving granuloma in an MTB infected tissue comparing to a level in a progressing granuloma in the MTB infected tissue. In some embodiments, the modulating comprises downregulating the expression of the one or more genes, wherein the one or more genes expresses at a lower level in a resolving granuloma in an MTB infected tissue comparing to a level in a progressing granuloma in the MTB infected tissue. In some embodiments, the modulating comprises delivering one or more agonists of the one or more genes to a subject.

In another aspect, the present disclosure includes engineered mast cells or plasmablasts comprising elevated expression of one or more genes that expresses at a level higher in a resolving granuloma in an MTB infected tissue comparing to a level in a progressing granuloma in the MTB infected tissue.

In another aspect, the present disclosure includes engineered mast cells or plasmablasts comprising reduced expression of one or more genes that expresses at a level lower in a resolving granuloma in an MTB infected tissue comparing to a level in a progressing granuloma in the MTB infected tissue.

In another aspect, the present disclosure includes methods of identifying a population of cells correlating to a granuloma characteristic, the method comprising: obtaining a first plurality of cells from one or more granuloma with the characteristic and a second plurality of cells from one or more granuloma without the characteristic; sequencing nucleic acid molecules in the first and the second pluralities of cells using single cell sequencing; clustering genes differently expressed between the first and the second plurality of cells; and identifying the population of cells based on the clustering of the different expressed genes.

In some embodiments, the methods further comprise excluding a cluster of genes expressing only in a single granuloma. In some embodiments, the methods further comprise identifying the population of cells in different subjects. In some embodiments, the granuloma characteristic is progressiveness.

In another aspect, the present disclosure includes methods of determining a characteristic of a granuloma in a subject infected by MTB, the method comprising identifying the population of cells according to methods disclosed herein.

In another aspect, the present disclosure includes methods of treatment or prophylaxis of a Mycobacterium tuberculosis (MTB) infection comprising activating a p53 pathway in macrophages of or from a patient in need thereof.

In another aspect, the present disclosure includes methods of treatment or prophylaxis of a Mycobacterium tuberculosis (MTB) infection comprising delivering a p53 agonist to a patient in need thereof.

In another aspect, the present disclosure includes methods of treatment or prophylaxis of a Mycobacterium tuberculosis (MTB) infection comprising delivering a p53 agonist to a patient's macrophages. In some embodiments, the p53 agonist is a p53 pathway agonist. In some embodiments, the p53 agonist is delivered to the patient's macrophages in vivo. In some embodiments, the macrophages are activated or agonized in vivo. In some embodiments, the macrophages are activated or agonized in a sample from the patient or ex vivo, and optionally, subsequently returned to the patient. In some embodiments, the p53 agonist is delivered to the patient's macrophages in a sample from the patient or ex vivo, and optionally, subsequently returned to the patient. In some embodiments, the p53 pathway activator, p53 agonist or p53 pathway agonist the p53 pathway activator, p53 agonist, or p53 pathway agonist is an MDM2 inhibitor. In some embodiments, the MDM2 inhibitor is nutilin-3a. In some embodiments, the control of MTB infection by macrophages in the patient is promoted. In some embodiments, the macrophage is, or is derived from, a primary human CD14⁺ monocyte-derived macrophage (MDM). In some embodiments, at least one of the following genes are upregulated: TOP2B, SORT1, NUDT3, IRF4, CXCL1.

In another aspect, the present disclosure includes methods of differentiating one or more macrophage subpopulations infected by MTB from one or more uninfected macrophage subpopulations, the method comprising: a. assaying the macrophages for the presence, or overexpression compared to wildtype macrophages, of: i. at least one of cytokine receptors (including IFNGR1, IL1RN), SLAM family members (including SLAM7, SLAMF5), and kinases (including HCK, CAMK1), or ii. at least one of differentiators of macrophage state, including M1 and M2, HLA-DRB1, and CD68, in particular CD86; b. assaying the macrophages for the presence, or overexpression compared to wildtype macrophages, of at least one of: i. at least one of ApoE, CD36, CD52, and IL8 ApoE, CD36, CD52, IL-8; c. identifying the one or more infected macrophage subpopulations based on the assay in a); d. identifying the one or more uninfected macrophage subpopulations based on the assay in b); and optionally, e. separating the one or more infected macrophage subpopulations from the one or more uninfected macrophage subpopulations based on the identifications made in c) and d); wherein separating optionally comprises i. by labelling or tagging one of the infected or the uninfected subpopulations; or ii. by differentially labelling or tagging the infected and the uninfected subpopulations. In some embodiments, identified, and optionally separated, infected macrophage subpopulations are contacted with a p53 agonist or p53 pathway agonist to promote a control phenotype.

In another aspect, the present disclosure includes methods of treatment of a Mycobacterium tuberculosis (MTB) infection, comprising activating the p53 pathway in macrophages of or from the patient to promote a control phenotype.

In another aspect, the present disclosure includes methods prophylaxis of a Mycobacterium tuberculosis (MTB) infection, comprising activating a p53 pathway in macrophages of or from a patient exposed to or at risk of MTB infection, optionally to promote a control phenotype.

In another aspect, the present disclosure includes methods of treatment or prophylaxis of an Mycobacterium tuberculosis (MTB) infection comprising activating a NF-κB pathway in macrophages of or from a patient in need thereof.

In another aspect, the present disclosure includes methods of treatment or prophylaxis of a Mycobacterium tuberculosis (MTB) infection comprising activating a Vitamin D Receptor (VDR) pathway in macrophages of or from a patient in need thereof.

In another aspect, the present disclosure includes a CD14⁺ macrophage model cell or cell line, wherein: at least one of the following genes are upregulated: CD206, CD86, CD32; and/or at least one of the following genes are downregulated: CD163. In some embodiments, the model cell or cell line is or is derived from a primary human CD14⁺ monocyte-derived macrophage (MDM).

In another aspect, the present disclosure provides a method of treating or preventing a disease by modulating a microenvironment of a cell or cell mass in a subject, the method comprising administrating an effective amount of one or more modulating agents that modulates mast cells, plasma cells, Th1-Th17 cells, and/or CD8+ T cells in the subject.

In some embodiments, the one or more modulating agents modulates expression of one or more genes in mast cells. In some embodiments, the modulating agent reduce number or function of mast cells. In some embodiments, the one or more modulating agents increase number or function of Th1-Th17 cells. In some embodiments, the one or more genes in mast cells comprises genes in IL-13 signaling pathway and/or genes in IL-33 signaling pathway. In some embodiments, the one or more genes in mast cells comprises IL-33, IL-1R1, and/or IL-13. In some embodiments, the one or more genes in Th1-Th17 cells. In some embodiments, the one or more genes in Th1-Th17 cells comprises genes in INF-γ signaling pathway and/or genes in TGFβ signaling pathway. In some embodiments, the one or more genes in Th1-Th17 cells comprises INF-γ, INF-γ receptor 2, TGFβ1, TGFβ receptor 3, CCL5, and/or IL-23R. In some embodiments, the one or more genes in Th1-Th17 cells comprises IL-2RG, IFN-γ, IFI27, LAG3, TIGIT, CD8A, NKG7, CCL20, CCL3, and/or CCL5. In some embodiments, the one or more modulating agents modulates expression of: a. GZMA, GZMB, GNLY, and PRF1, b. CCR7, LEF1, and SELL in Naïve T cells, c. FOXP3, IKZF2, and IL1RL1 in regulatory T cells, d. OAS2, MX1, and ISG15 in interferon-responsive cells, e. GZMK, CCL5, and CXCR4 in CD8+ T cells, f. CX3CR1, GZMB, and ZEB2 in CD8+ T cells, g. MKI67 and TOP2A in proliferating T cells, h. APOC1, APOE, and C1QB in alveolar macrophages, i. TIMP1 and IDO1 in monocytes, j. LIPA and MAN2B1 in macrophages, k. MRC1, FABP5, and PPARG in lipid-laden macrophages, 1. CP, CXCL9, and NFKB1 in inflammatory macrophages, m. MKI67 and TOP2A in proliferating macrophages, n. BIRC3, CCR7, and LAMP3 in myeloid dendritic cells, o. BHLHE40, SATB1 and RBPJ in Th17 cells, p. IFNG, CCL4, RORC, IL17A, IL17F, IL1R1, RORA, IRF4, and RBPJ in Th17 cells, q. IL23R, IL7R, NDFIP1, ILI1R1, RORA, IRF4, and RBPJ in Ex-Th17 cells, r. KLF2, TGFBR3, CX3CR1, and GZMB in CD8+ T cells, s. FOXP3, TIGIT, GITR, and GATA3 in ST2+ regulatory T cells, t. IL2RG, IFNG, IFI27, LAG3, TIGIT, CD8A, NKG7, CCL20, CCL3, and CCL5 in Th1-Th17 cell, or u. any combination thereof.

In some embodiments, the one or more modulating agents is comprised in a vaccine formulation. In some embodiments, the disease is bacterial infection, tuberculosis, cancer, chronic rhinosinusitis, asthma, allergy, wound, or a combination thereof. In some embodiments, the disease is a latent disease. In some embodiments, the disease is an active disease. In some embodiments, the cell or cell mass is a granuloma. In some embodiments, the one or more modulating agents comprises an antibody, or antigen binding fragment, an aptamer, affimer, non-immunoglobulin scaffold, small molecule, genetic modifying agent, or a combination thereof.

In another aspect, the present disclosure provides a method of treating a disease in a subject comprising: a. contacting one or more mast cells and/or Th1-Th17 cells with one or more modulating agents, wherein the one or more modulating agents activates i. IL-33, IL-1R1, and genes in IL-13 signaling pathway in the mast cells, and/or ii. INF-γ, INF-γ receptor 2, TGFβ1, TGFβ receptor 3, CCL5, and genes in INF-γ and TGFβ signaling pathway in the Th1-Th17 cells; b. administering the mast cells and/or Th1-Th17 from a) to the subject. In some embodiments, the mast cells and/or Th1-Th17 cells are isolated or derived from the subject.

In another aspect, the present disclosure provides a mast cell or cell line expressing one or more of: IL-33, IL-1R1, and genes in IL-13 signaling pathway.

In another aspect, the present disclosure provides a Th1-Th17 cell expressing one or more of: INF-γ, INF-γ receptor 2, TGFβ1, TGFβ receptor 3, CCL5, and genes in INF-γ and TGFβ signaling pathway.

In another aspect, the present disclosure provides a vaccine comprising the one or more modulating agents herein.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1F show development an in vitro model of Mtb infection.

FIGS. 2A-2E show single cell RNA sequencing and analysis of cells from granulomas.

FIGS. 3A-3H show characterization of macrophages in granulomas.

FIGS. 4A-4F show analysis of gene expression in macrophages in granulomas.

FIG. 5 shows an exemplary method for unbiased definition of the features of restrictive and permissive granulomas.

FIG. 6 shows analysis of the single cell RNAseq results.

FIG. 7 shows Tuberculosis (TB) granuloma atlas.

FIG. 8 shows identification of T cell clusters in NHP with early progression in disease.

FIG. 9 shows cell type identification.

FIG. 10 shows identification of T cell clusters and correlation with control in an NHP with early progression.

FIG. 11 shows identification of T cell clusters and correlation with control in another NHP with early progression.

FIG. 12 shows composition of T cell compartment varies across hosts.

FIG. 13 shows exploration of cell populations that had correlation with progressiveness shared across hosts.

FIG. 14 shows strong correlation in all animals between progressiveness and mast cells or plasmablasts.

FIG. 15 shows an exemplary method used herein.

FIG. 16 shows exemplary serial PET-CT.

FIG. 17 shows an exemplary test for granuloma-level end-point bacterial burden.

FIG. 18 shows an exemplary test for cumulative bacterial burden.

FIGS. 19-21 show exemplary tests for generic cell type identification.

FIGS. 22-23 show exemplary tests for T cell phenotypes.

FIG. 24 shows comparison of the gene signatures in an exemplary method herein with reported signatures.

FIGS. 25-27 show exemplary tests for analyzing normal lungs.

FIGS. 28-29 show exemplary tests for macrophage sub-clusters.

FIGS. 30-32 show exemplary tests for cell-type CFU relationships.

FIGS. 33-34 show exemplary tests for macrophage CFU relationships.

FIG. 35 shows exemplary tests for CFU-CEQ relationships.

FIGS. 36-39 show exemplary tests for T cell phenotypic compositions.

FIG. 40 shows an exemplary test for the distribution of genes that distinguished Th17 and Th1-Th17 cells.

FIG. 41 shows an exemplary test for relationship between effector CD8:Treg ratio to CFU.

FIG. 42 shows an exemplary test for the distribution of canonical T cell signature genes.

FIG. 43 shows an exemplary test for the distribution of Th1 signature genes.

FIG. 44 shows an exemplary test for the distribution of Th2 signature genes.

FIG. 45 shows an exemplary test for the distribution of Th17 signature genes.

FIG. 46 shows an exemplary test for the distribution of T cell exhaustion gene expression by cluster.

FIG. 47 shows an exemplary test for the distribution of exhaustion signatures from Miller et al. Nature Immunology 2019.

FIGS. 48-49 show exemplary tests for signatures of T cell exhaustion.

FIGS. 50-51 show DE of T cell exhaustion gene between high and low burden lesions.

FIG. 52 shows an exemplary test for the distribution of cytotoxic gene expression.

FIG. 53 shows an exemplary cytotoxic expression score.

FIG. 54 shows an exemplary test for Th1-Th17 differential expression.

FIG. 55 shows an exemplary test for NK differential expression.

FIG. 56 shows an exemplary test for effector CD8 differential expression.

FIG. 57 shows an exemplary test for the distribution of expanded TCR clones.

FIG. 58 shows an exemplary test for the clonal sharing across granulomas.

FIG. 59 shows an exemplary test for the clonal sharing across T cell phenotypes.

FIG. 60 shows an exemplary test for the relationship between granuloma age and CFU.

FIG. 61 shows an exemplary test for T cell composition and timing of formation.

FIG. 62 shows an exemplary test for the T cell-CFU relationship, original lesions, highest vs. lowest.

FIG. 63 shows an exemplary test for granuloma ecology.

FIG. 64 shows an exemplary method for generation of receptor-ligand interaction networks.

FIGS. 65-67 show exemplary tests for receptor-ligand interactions increased in high burden lesions.

FIGS. 68-69 show exemplary tests for receptor-ligand interactions increased in low burden lesions.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

In one aspect, embodiments disclosed herein leverage natural control of tuberculosis (TB) infection leveraging cellular pathways and intercellular communications that promote control and/or turn of cellular pathways and intercellular communications that promote persistence and/or expansion of the infection. As shown herein outcomes of TB infection are not homogenous but instead is heterogenous on multiple scales. Single cell approaches have been applied allowing for an unbiased dissection of immune heterogeneity related to TB infection.

In another aspect, the present disclosure provides methods of treating or preventing diseases by modulating microenvironment of a cell or cell mass in a subject. In some embodiments, the methods comprise administering one or more modulating agents (interchangeably used with “agent” herein) to modulate the number and/or function of one or more types of cells (e.g., immune cells) in the subject. In some embodiments, the methods comprise reducing the number and/or function of mast cells. In some embodiments, the methods comprise increasing the number and/or function of Th1-Th17 cells. The modulation of the number and/or function of the cells may occur in desired cell masses or tissues. The modulation of the number and/or function of the cells may regulate host control of bacterial infection.

### Methods of Treatment

In certain embodiments, a method of treatment or prophylaxis of a Mycobacterium tuberculosis (Mtb) infection comprises activating a p53 pathway in macrophages of or from a patient in need thereof. In certain example embodiments, the method comprises delivering a p53 agonist to a patient in need thereof. In certain other example embodiments, the p53 agonist may be delivered to a patient's macrophages in vivo or ex vivo. In certain other embodiments, a method of treatment or prophylaxis of a Mycobacterium tuberculosis (Mtb) infection comprises activating a NF-kB agonist. Examples of NF-kB agonists include betulinic acid, prostratin, PMA, and calcimycin. In certain example embodiments, the method comprises delivering a NF-kB agonist to a patient in need thereof. In certain other example embodiments, the NF-kB agonist may be delivered to a patient's macrophages in vivo or ex vivo. In certain example embodiments, a method of treatment or prophylaxis of a Mycobacterium tuberculosis (Mtb) infection comprises activating a Vitamin D Receptor pathway in macrophages of or from a patient in need thereof. In certain example embodiments, the method comprises delivering a vitamin D receptor pathway agonist to a patient in need thereof. In certain other example embodiments, the vitamin D receptor agonist may be delivered to a patient's macrophages in vivo or ex vivo.

In some embodiments, the methods for treatment or prophylaxis of a Mycobacterium tuberculosis (MTB) infection comprise modulating expression of one or more genes in a mast cell, a plasmablast, or a combination thereof, in a subject in need thereof, wherein the one or more genes expresses at a level different in a resolving granuloma in an MTB infected tissue comparing to a level in a progressing granuloma in the MTB infected tissue. In some embodiments, the methods of identifying a population of cells correlating to a granuloma characteristic comprise: obtaining a first plurality of cells from one or more granuloma with the characteristic and a second plurality of cells from one or more granuloma without the characteristic; sequencing nucleic acid molecules in the first and the second pluralities of cells using single cell sequencing; clustering genes differently expressed between the first and the second plurality of cells; and identifying the population of cells based on the clustering of the different expressed genes.

In some embodiments, the present disclosure provides methods for differentiating one or more macrophage subpopulations infected by MTB from one or more uninfected macrophage subpopulations. In some examples, the methods comprise: assaying the macrophages for the presence, or overexpression compared to wildtype macrophages, of: at least one of cytokine receptors (including IFNGR1, IL1RN), SLAM family members (including SLAM7, SLAMF5), and kinases (including HCK, CAMK1), or at least one of differentiators of macrophage state, including M1 and M2, HLA-DRB1, and CD68, in particular CD86; assaying the macrophages for the presence, or overexpression compared to wt macrophages, of at least one of: at least one of ApoE, CD36, CD52, and IL-8 ApoE, CD36, CD52, IL8, identifying the one or more infected macrophage subpopulations based on the assay in a); identifying the one or more uninfected macrophage subpopulations based on the assay in b); and optionally, separating the one or more infected macrophage subpopulations from the one or more uninfected macrophage subpopulations based on the identifications made in c) and d); wherein separating optionally comprises by labelling or tagging one of the infected or the uninfected subpopulations; or by differentially labelling or tagging the infected and the uninfected subpopulations.

In some embodiments, the present disclosure provides methods of treating or preventing diseases by modulating a microenvironment of a cell or cell mass. The methods may comprise administering one or more modulating agents to modulate the number and/or function of certain types of cells. In some examples, the methods comprise administrating an effective amount of one or more modulating agents that modulates mast cells, plasma cells, Th1-Th17 cells, and/or CD8+ T cells in the subject.

In some embodiments, the present disclosure provides cells or cell lines in which one or more genes is modulated. Such modulation include expression of the one or more genes. The expression of the gene(s) by the modulation may be higher than a counterpart wildtype cell or cell line. Alternatively or additionally, the modulation include suppression of the one or more genes. The suppression of the gene(s) by the modulation may be cause lower expression of the genes compared to a counterpart wildtype cell or cell line. In some cases, the present disclosure provides mast cell or cell lines in which one or more genes is modulated. For examples, the mast cell or cell lines may express one or more target genes herein, e.g., expressing one or more of: IL-33, IL-1R1, and genes in IL-13 signaling pathway. In some cases, the present disclosure provides Th1-Th17 cell or cell lines in which one or more genes is modulated. For examples, the Th1-Th17 cell or cell lines may express one or more target genes herein, e.g., INF-γ, INF-γ receptor 2, TGFβ1, TGFβ receptor 3, CCL5, and genes in INF-γ and TGFβ signaling pathway.

In some embodiments, the modulation of genes or protein may be increasing the expression, activities, and/or stability of the genes or proteins. In certain embodiments, the modulation of genes or protein may be decreasing the expression, activities, and/or stability of the genes or proteins.

### Target Genes and Proteins

The methods herein may comprise modulating the expression and/or function of the one or more target genes and proteins.

### Microenvironment

In some embodiments, altering the expression and/or function of the target genes and proteins may modulate the microenvironment in a cell, cell mass, or tissue in a subject. Microenvironment may refer the sum total of cell-cell, cell-ECM, and cell-soluble factor interactions, in addition geometric and physical properties of the surroundings, that are experienced by a cell. Cell-microenvironment interactions may make possible the levels of tissue specific behavior observed in every higher organism, where there may be billions of cells with identical genetic information that serve as constituents of the different tissues and organs. In order for each tissue or organ to operate successfully within the context of the organism, all cells must be integrated into an architectural and signaling framework such that each cell knows which commands to execute at any given time. Success at this daunting task leads to homeostasis, whereas failure results in a spectrum of dysfunctions, including cancer, other diseases, and aging. Microenvironment properties combine to exert control over the genome in both normal and diseased cells. Isolated cells are known to lose most functional differentiation when separated and placed in traditional cell cultures. However, the cellular identity is not lost permanently, as this can be achieved by controlling the microenvironment of the cells in culture, the cell can “remember” many of their original tissue specific traits. Metastable epigenetic states of cells also may be essential to help maintain the fidelity of phenotypes that are the result of dynamic and reciprocal interactions between cells and their microenvironments.

### Signature Genes/Proteins

The target genes or proteins may be signature genes or proteins of certain types of cells. By modulating the signature genes, the levels and/or activities of one or more types of cells may be modulated.

The signatures or signature genes herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures may be discovered by analysis of expression profiles of single-cells within a population of cells from isolated samples (e.g. tumor samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type.

The signature may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular cell or cell (sub)population state if it is upregulated or only present, detected or detectable in that particular cell or cell (sub)population state (e.g., disease or healthy), or alternatively is downregulated or only absent, or undetectable in that particular cell or cell (sub)population state. In this context, a signature consists of one or more differentially expressed genes/proteins or differential epigenetic elements when comparing different cells or cell (sub)populations, including comparing different gut cell or gut cell (sub)populations, as well as comparing gut cell or gut cell (sub)populations with healthy or disease (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population or subpopulation level, refer to genes that are differentially expressed in all or substantially all cells of the population or subpopulation (such as at least 80%, preferably at least 90%, such as at least 95% of the individual cells). This allows one to define a particular subpopulation of immune cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferably it is meant: induction or alternatively suppression (or upregulation or downregulation) of at least one gene/protein and/or epigenetic element of the signature, such as for instance at least two, at least three, at least four, at least five, at least six, or all genes/proteins and/or epigenetic elements of the signature.

### Examples of Target Genes

The target genes and proteins may include p53 and those in p53 signaling pathway. In some embodiments, examples of target genes include TOP2B, SORT1, NUDT3, IRF4, CXCL1, cytokine receptors (including IFNGR1, IL1RN), SLAM family members (including SLAM7, SLAMF5), and kinases (including HCK, CAMK1), or differentiators of macrophage state, including M1 and M2, HLA-DRB1, and CD68, in particular CD86, ApoE, CD36, CD52, and IL-8 ApoE, CD36, CD52, IL8, CD206, CD86, CD32; CD163. Such genes may be in macrophages, e.g., CD4+ macrophages.

In some embodiments, the target genes may be those in the IL-13 signaling pathway and/or the IL-33 pathway. In certain examples, the target genes include IL-33, IL-1R1, and IL-13. In some embodiments, these genes may be modulated in mast cells.

In some embodiments, the target genes may be those in the INF-γ signaling pathway and/or genes in TGFβ signaling pathway. In certain examples, the target genes include INF-γ, INF-γ receptor 2, TGFβ1, TGFβ receptor 3, CCL5, and IL-23R. In certain examples, the target genes include IL-2RG, IFN-γ, IFI27, LAG3, TIGIT, CD8A, NKG7, CCL20, CCL3, and CCL5. In some embodiments, these genes may be modulated in Th1-Th17 cells.

In some embodiments, the target genes include GZMA, GZMB, GNLY, and PRF1. In some embodiments, the target genes include CCR7, LEF1, and SELL (e.g., modulated in Naïve T cells). In some embodiments, the target genes include FOXP3, IKZF2, and IL1RL1 (e.g., modulated in regulatory T cells). In some embodiments, the target genes include OAS2, MX1, and ISG15 (e.g., modulated in interferon-responsive cells). In some embodiments, the target genes include GZMK, CCL5, and CXCR4 (e.g., modulated in CD8+ T cells). In some embodiments, the target genes include CX3CR1, GZMB, and ZEB2 (e.g., modulated in CD8+ T cells). In some embodiments, the target genes include MKI67 and TOP2A (e.g., modulated in proliferating T cells). In some embodiments, the target genes include APOC1, APOE, and C1QB (e.g., modulated in alveolar macrophages). In some embodiments, the target genes include TIMP1 and IDO1 (e.g., modulated in monocytes). In some embodiments, the target genes include LIPA and MAN2B1 (e.g., modulated in macrophages). In some embodiments, the target genes include MRC1, FABP5, and PPARG (e.g., modulated in lipid-laden macrophages). In some embodiments, the target genes include CP, CXCL9, and NFKB1 (e.g., modulated in inflammatory macrophages). In some embodiments, the target genes include MKI67 and TOP2A (e.g., modulated in proliferating macrophages). In some embodiments, the target genes include BIRC3, CCR7, and LAMP3 (e.g., modulated in myeloid dendritic cells). In some embodiments, the target genes include BHLHE40, SATB1 and RBPJ (e.g., modulated in Th17 cells). In some embodiments, the target genes include IFNG, CCL4, RORC, IL17A, IL17F, IL1R1, RORA, IRF4, and RBPJ (e.g., modulated in Th17 cells). In some embodiments, the target genes include IL23R, IL7R, NDFIP1, ILI1R1, RORA, IRF4, and RBPJ (e.g., modulated in Ex-Th17 cells). In some embodiments, the target genes include KLF2, TGFBR3, CX3CR1, and GZMB (e.g., modulated in CD8+ T cells). In some embodiments, the target genes include FOXP3, TIGIT, GITR, and GATA3 (e.g., modulated in ST2+ regulatory T cells). In some embodiments, the target genes include IL2RG, IFNG, IFI27, LAG3, TIGIT, CD8A, NKG7, CCL20, CCL3, and CCL5 (e.g., modulated in Th1-Th17 cell). In some embodiments, the target genes HIF-1 and those in HIF-1 signaling pathway.

### Contacting or Delivering

The methods comprise contacting and/or delivering one or more modulating agents to a subject. The contacting may take place in vitro, in vivo, ex vivo. In some instances, contacting can be performed by incubating a cell or tissue having a certain phenotype with the candidate modulating agent. In some instances, contacting can be performed by delivering the candidate modulating agent to a subject in need thereof. The step of contacting is performed under conditions and for a time sufficient to allow the modulating agent and the cell, tissue, gene, or gene product to interact.

In some embodiments, the cells or population of cells may be obtained from a biological sample. The biological sample may be obtained from a subject suffering from a disease. The biological sample may be a granuloma sample from a subject suffering from tuberculosis infection.

As used herein, a “biological sample” may contain whole cells and/or tissue and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

### Agonists

The modulating agents described below may be a p53-pathway, NF-kB pathway, or a vitamin D receptor pathway agonist, and can be any composition that induces, represses, or otherwise affects a gene or gene product in said pathways. Modulating agents may be selected in some instances, based on a particular pathway, degree of infection, and/or a gene expression signature that may have been detected. In certain example embodiments, p53 pathway agonist may be a MDM2 inhibitor. In certain example embodiments, the p53 pathway agonist may be one of the MDM2 antagonist described in Vassilev et al. Science 303, 844-848 (2004) or Chene, Nat. Rev. Cancer 3, 102-109 (2003). In certain example embodiments, the MDM2 antagonist is nutlin3-a.

As used herein, modulating, or to modulate, generally means either reducing or inhibiting the expression or activity of, or alternatively increasing the expression or activity of a target gene. In particular, modulating can mean either reducing or inhibiting the activity of, or alternatively increasing a (relevant or intended) biological activity of, a target or antigen as measured using a suitable in vitro, cellular or in vivo assay (which will usually depend on the target involved), by at least 5%, at least 10%, at least 25%, at least 50%, at least 60%, at least 70%, at least 80%, at least 90%, or more, compared to activity of the target in the same assay under the same conditions but without the presence of an agent. An increase or decrease refers to a statistically significant increase or decrease respectively. For the avoidance of doubt, an increase or decrease will be at least 10% relative to a reference, such as at least 10%, at least 20%, at least 30%, at least 40%, at least 50%, at least 60%, at least 70%, at least 80%, at least 90%, at least 95%, at least 97%, at least 98%, or more, up to and including at least 100% or more, in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more. Modulating can also involve effecting a change (which can either be an increase or a decrease) in affinity, avidity, specificity and/or selectivity of a target or antigen, such as a receptor and ligand. Modulating can also mean effecting a change with respect to one or more biological or physiological mechanisms, effects, responses, functions, pathways or activities in which the target or antigen (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist can be determined in any suitable manner and/or using any suitable assay known or described herein (e.g., in vitro or cellular assay), depending on the target or antigen involved. Accordingly, a modulating agent in an amount sufficient to modify a mycobacteria infection in a cell or tissue would provide the agent in an amount to effect a change in the amount of infection compared to the amount of infection in the cell or tissue in the absence of modulating agent, or untreated. The amount of modulating agent will vary according to the pathway, gene, or gene product targeted, the host, the tissue or cell, and the amount or copy number of the mycobacteria infection.

Modulating can, for example, also involve allosteric modulation of the target and/or reducing or inhibiting the binding of the target to one of its substrates or ligands and/or competing with a natural ligand, substrate for binding to the target. Modulating can also involve activating the target or the mechanism or pathway in which it is involved. Modulating can for example also involve effecting a change in respect of the folding or confirmation of the target, or in respect of the ability of the target to fold, to change its conformation (for example, upon binding of a ligand), to associate with other (sub)units, or to disassociate. Modulating can for example also involve effecting a change in the ability of the target to signal, phosphorylate, dephosphorylate, and the like.

### Protein Binding Agents

The modulating agents may be protein binding agents. As used herein, an agent can refer to a protein-binding agent that permits modulation of activity of proteins or disrupts interactions of proteins and other biomolecules, such as but not limited to disrupting protein-protein interaction, ligand-receptor interaction, or protein-nucleic acid interaction. Agents can also refer to DNA targeting or RNA targeting agents. Agents may include a fragment, derivative and analog of an active agent. The terms “fragment,” “derivative” and “analog” when referring to polypeptides as used herein refers to polypeptides which either retain substantially the same biological function or activity as such polypeptides. An analog includes a proprotein which can be activated by cleavage of the proprotein portion to produce an active mature polypeptide. Such agents include, but are not limited to, antibodies (“antibodies” includes antigen-binding portions of antibodies such as epitope- or antigen-binding peptides, paratopes, functional CDRs; recombinant antibodies; chimeric antibodies; humanized antibodies; nanobodies; tribodies; midibodies; or antigen-binding derivatives, analogs, variants, portions, or fragments thereof), protein-binding agents, nucleic acid molecules, small molecules, recombinant protein, peptides, aptamers, avimers and protein-binding derivatives, portions or fragments thereof. An “agent” as used herein, may also refer to an agent that inhibits expression of a gene, such as but not limited to a DNA targeting agent (e.g., CRISPR system, TALE, Zinc finger protein) or RNA targeting agent (e.g., inhibitory nucleic acid molecules such as RNAi, miRNA, ribozyme).

The agents of the present invention may be modified, such that they acquire advantageous properties for therapeutic use (e.g., stability and specificity), but maintain their biological activity.

The properties of certain proteins can be modulated by attachment of polyethylene glycol (PEG) polymers, which increases the hydrodynamic volume of the protein and thereby slows its clearance by kidney filtration. (See, e.g., Clark et al., J. Biol. Chem. 271: 21969-21977 (1996)). Therefore, it is envisioned that certain agents can be PEGylated (e.g., on peptide residues) to provide enhanced therapeutic benefits such as, for example, increased efficacy by extending half-life in vivo. In certain embodiments, PEGylation of the agents may be used to extend the serum half-life of the agents and allow for particular agents to be capable of crossing the blood-brain barrier.

In regards to peptide PEGylation methods, reference is made to Lu et al., Int. J. Pept. Protein Res. 43: 127-38 (1994); Lu et al., Pept. Res. 6: 140-6 (1993); Felix et al., Int. J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., hit. J. Hematol. 68: 1-18 (1998); Roberts et al., J. Pharm. Sci. 87: 1440-45 (1998); and Tan et al., Protein Expr. Purif 12: 45-52 (1998). Polyethylene glycol or PEG is meant to encompass any of the forms of PEG that have been used to derivatize other proteins, including, but not limited to, mono-(C1-10) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG2-NHS-40k (Nektar); mPEG2-MAL-40k (Nektar), SUNBRIGHT GL2-400MA ((PEG)240 kDa) (NOF Corporation, Tokyo), SUNBRIGHT ME-200MA (PEG20 kDa) (NOF Corporation, Tokyo). The PEG groups are generally attached to the peptide (e.g., neuromedin U receptor agonists or antagonists) via acylation or alkylation through a reactive group on the PEG moiety (for example, a maleimide, an aldehyde, amino, thiol, or ester group) to a reactive group on the peptide (for example, an aldehyde, amino, thiol, a maleimide, or ester group).

The PEG molecule(s) may be covalently attached to any Lys, Cys, or K(CO(CH2)2SH) residues at any position in a peptide. In certain embodiments, the neuromedin U receptor agonists described herein can be PEGylated directly to any amino acid at the N-terminus by way of the N-terminal amino group. A “linker arm” may be added to a peptide to facilitate PEGylation. PEGylation at the thiol side-chain of cysteine has been widely reported (see, e.g., Caliceti & Veronese, Adv. Drug Deliv. Rev. 55: 1261-77 (2003)). If there is no cysteine residue in the peptide, a cysteine residue can be introduced through substitution or by adding a cysteine to the N-terminal amino acid.

Substitutions of amino acids may be used to modify an agent of the present invention. The phrase “substitution of amino acids” as used herein encompasses substitution of amino acids that are the result of both conservative and non-conservative substitutions. Conservative substitutions are the replacement of an amino acid residue by another similar residue in a polypeptide. Typical but not limiting conservative substitutions are the replacements, for one another, among the aliphatic amino acids Ala, Val, Leu and Ile; interchange of Ser and Thr containing hydroxy residues, interchange of the acidic residues Asp and Glu, interchange between the amide-containing residues Asn and Gln, interchange of the basic residues Lys and Arg, interchange of the aromatic residues Phe and Tyr, and interchange of the small-sized amino acids Ala, Ser, Thr, Met, and Gly. Non-conservative substitutions are the replacement, in a polypeptide, of an amino acid residue by another residue which is not biologically similar. For example, the replacement of an amino acid residue with another residue that has a substantially different charge, a substantially different hydrophobicity, or a substantially different spatial configuration.

Antibody is used interchangeably with the term immunoglobulin herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

An antigen-binding fragment refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term antibody encompass any Ig class or any Ig subclass (e.g. the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or immunoglobulin class, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

IgG subclass refers to the four subclasses of immunoglobulin class IgG—IgG1, IgG2, IgG3, and IgG4 that have been identified in humans and higher mammals by the heavy chains of the immunoglobulins, V1-γ4, respectively. The term single-chain immunoglobulin or single-chain antibody (used interchangeably herein) refers to a protein having a two-polypeptide chain structure consisting of a heavy and a light chain, said chains being stabilized, for example, by interchain peptide linkers, which has the ability to specifically bind antigen. The term domain refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The variable domains of an antibody heavy chain are referred to interchangeably as heavy chain constant regions, heavy chain constant domains, “VH” regions or “VH” domains).

A region can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

Conformation refers to the tertiary structure of a protein or polypeptide (e.g., an antibody, antibody chain, domain or region thereof). For example, light (or heavy) chain conformation can refer to the tertiary structure of a light (or heavy) chain variable region, and the antibody conformation or antibody fragment conformation refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from non-immunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant cross reactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly cross react with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having VHand C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a blocking antibody or an antibody antagonist is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its downstream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The modulating agent may be receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Nucleic Acid Molecules

The modulating agents may be nucleic acid molecules, in particular those that inhibit a target gene. Exemplary nucleic acid molecules include aptamers, siRNA, artificial microRNA, interfering RNA or RNAi, dsRNA, ribozymes, antisense oligonucleotides, and DNA expression cassettes encoding said nucleic acid molecules. Preferably, the nucleic acid molecule is an antisense oligonucleotide. Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Preferred ASOs include Locked Nucleic Acid (LNA), Peptide Nucleic Acid (PNA), and morpholinos Preferably, the nucleic acid molecule is an RNAi molecule, i.e., RNA interference molecule. Preferred RNAi molecules include siRNA, shRNA, and artificial miRNA. The design and production of siRNA molecules is well known to one of skill in the art (e.g., Hajeri P B, Singh S K. Drug Discov Today. 2009 14(17-18):851-8). The nucleic acid molecule inhibitors may be chemically synthesized and provided directly to cells of interest. The nucleic acid compound may be provided to a cell as part of a gene delivery vehicle. Such a vehicle is preferably a liposome or a viral gene delivery vehicle.

### Small Molecules

In certain embodiments, the one or more modulating agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000 Da, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810). Accordingly, in certain embodiments, the one or more agents comprises a small molecule inhibitor, small molecule degrader (e.g., PROTAC), genetic modifying agent, antibody, antibody fragment, antibody-like protein scaffold, aptamer, protein, or any combination thereof.

As described herein, small molecules targeting epigenetic proteins are currently being developed and/or used in the clinic to treat disease (see, e.g., Qi et al., HEDD: the human epigenetic drug database. Database, 2016, 1-10; and Ackloo et al., Chemical probes targeting epigenetic proteins: Applications beyond oncology. Epigenetics 2017, VOL. 12, NO. 5, 378-400). In certain embodiments, the one or more agents comprise a histone acetylation inhibitor, histone deacetylase (HDAC) inhibitor, histone lysine methylation inhibitor, histone lysine demethylation inhibitor, DNA methyltransferase (DNMT) inhibitor, inhibitor of acetylated histone binding proteins, inhibitor of methylated histone binding proteins, sirtuin inhibitor, protein arginine methyltransferase inhibitor or kinase inhibitor. In certain embodiments, any small molecule exhibiting the functional activity described above may be used in the present invention. In certain embodiments, the DNA methyltransferase (DNMT) inhibitor is selected from the group consisting of azacitidine (5-azacytidine), decitabine (5-aza-2′-deoxycytidine), EGCG (epigallocatechin-3-gallate), zebularine, hydralazine, and procainamide. In certain embodiments, the histone acetylation inhibitor is C646. In certain embodiments, the histone deacetylase (HDAC) inhibitor is selected from the group consisting of vorinostat, givinostat, panobinostat, belinostat, entinostat, CG-1521, romidepsin, ITF-A, ITF-B, valproic acid, OSU-HDAC-44, HC-toxin, magnesium valproate, plitidepsin, tasquinimod, sodium butyrate, mocetinostat, carbamazepine, SB939, CHR-2845, CHR-3996, JNJ-26481585, sodium phenylbutyrate, pivanex, abexinostat, resminostat, dacinostat, droxinostat, and trichostatin A (TSA). In certain embodiments, the histone lysine demethylation inhibitor is selected from the group consisting of pargyline, clorgyline, bizine, GSK2879552, GSK-J4, KDM5-C70, JIB-04, and tranylcypromine. In certain embodiments, the histone lysine methylation inhibitor is selected from the group consisting of EPZ-6438, GSK126, CPI-360, CPI-1205, CPI-0209, DZNep, GSK343, EI1, BIX-01294, UNC0638, EPZ004777, GSK343, UNC1999 and UNC0224. In certain embodiments, the inhibitor of acetylated histone binding proteins is selected from the group consisting of AZD5153 (see e.g., Rhyasen et al., AZD5153: A Novel Bivalent BET Bromodomain Inhibitor Highly Active against Hematologic Malignancies, Mol Cancer Ther. 2016 November; 15(11):2563-2574. Epub 2016 Aug. 29), PFI-1, CPI-203, CPI-0610, RVX-208, OTX015, I-BET151, I-BET762, I-BET-726, dBET1, ARV-771, ARV-825, BETd-260/ZBC260 and MZ1. In certain embodiments, the inhibitor of methylated histone binding proteins is selected from the group consisting of UNC669 and UNC1215. In certain embodiments, the sirtuin inhibitor comprises nicotinamide.

As detailed herein, modulate broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. The modulating agents can be used in an amount sufficient to modify an infection, a change in the amount or degree of infection as compared to in the absence of infection. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of a cell, cell population, or tissue, or any other infected cell or tissue may be modulated without substantially altering other (unintended, undesired) phenotypic aspect(s).

As further detailed herein, a modulating agent broadly encompasses any condition, substance or agent capable of modulating one or more phenotypic aspects of cell or cell population as disclosed herein. Such conditions, substances or agents may be of physical, chemical, biochemical and/or biological nature. A candidate agent refers to any condition, substance or agent that is being examined for the ability to modulate one or more phenotypic aspects of a cell or cell population as disclosed herein in a method comprising applying the candidate agent to the cell or cell population (e.g., exposing the cell, cell population, or tissue to the candidate agent or contacting the cell, cell population, or tissue with the candidate agent) and observing whether the desired modulation takes place. In some instances, the cell population comprises immune cells, in some embodiments, the cell population comprises macrophages.

Agents may include any potential class of biologically active conditions, substances or agents, such as for instance antibodies, proteins, peptides, nucleic acids, oligonucleotides, small molecules, or combinations thereof.

By means of example but without limitation, agents can include low molecular weight compounds, but may also be larger compounds, or any organic or inorganic molecule effective in the given situation, including modified and unmodified nucleic acids such as antisense nucleic acids, RNAi, such as siRNA or shRNA, CRISPR/Cas systems, peptides, peptidomimetics, receptors, ligands, and antibodies, aptamers, polypeptides, nucleic acid analogues or variants thereof. Examples include an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. Agents can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), modified RNA (mod-RNA), single guide RNA etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides, CRISPR guide RNA, for example that target a CRISPR enzyme to a specific DNA target sequence etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a gene within the cell. In some embodiments, the agent is any chemical, entity or moiety, including without limitation synthetic and naturally-occurring non-proteinaceous entities. In certain embodiments, the agent is a small molecule having a chemical moiety. Agents can be known to have a desired activity and/or property, or can be selected from a library of diverse compounds.

### Genetic Modulating Agents

The modulating agents herein may be one or more genetic modification agents. The genetic modulating agents may manipulate nucleic acids (e.g., genomic DNA or mRNA).

### Gene Editing Systems

In some embodiments, the modulating agents may be gene editing systems or components thereof. Examples of gene editing systems include CRISPR-Cas systems, zinc finger nuclease systems, TALEN systems, and meganuclease systems.

### CRISPR-Cas System

The modulating agents may be one or more components of a CRISPR-Cas system. For example, the nucleotide sequences may be or encode guide RNAs. The modulating agents may also encode Cas proteins, variants thereof, or fragments thereof.

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas protein (or interchangeably referred as CRISPR effector protein, CRISPR effector, CRISPR protein, Cas effector protein, or Cas effector), a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

### Cas Effector Proteins

In some examples, a CRISPR-Cas system comprises a Cas effector protein and guide RNA. Examples of Cas proteins include those of Class 1 (e.g., Type I, Type III, and Type IV) and Class 2 (e.g., Type II, Type V, and Type VI) Cas proteins, e.g., Cas9, Cas12 (e.g., Cas12a, Cas12b, Cas12c, Cas12d), Cas13 (e.g., Cas13a, Cas13b, Cas13c, Cas13d), CasX, CasY, Cas14, variants thereof (e.g., mutated forms, truncated forms), homologs thereof, and orthologs thereof. In some examples, the Cas effector protein is Cas9. In some examples, the Cas effector protein is Cas12. In some examples, the Cas effector protein is Cas13. Additional effectors for use according to the invention can be identified by their proximity to cas1 genes, for example, though not limited to, within the region 20 kb from the start of the cas1 gene and 20 kb from the end of the cas1 gene. Other examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof.

The modulating agents may comprise one or more Cas proteins or nucleic acids encoding thereof. The Cas proteins may be CRISPR RNA-Targeting Effector Proteins. In one example embodiment, the CRISPR system effector protein is an RNA-targeting effector protein. In certain embodiments, the CRISPR system effector protein is a Type VI CRISPR system targeting RNA (e.g., Cas13a, Cas13b, Cas13c or Cas13d). Example RNA-targeting effector proteins include Cas13b and C2c2 (now known as Cas13a).

In certain example embodiments, a Cas effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of Orthologous proteins may but need not be structurally related, or are only partially structurally related.

In some embodiments, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the nucleotide sequence may comprise a coding sequence for a CRISPR effector protein. The CRISPR effector protein may be delivered using a nucleic acid molecule encoding the CRISPR effector protein. The coding sequence for a CRISPR effector protein may be a codon optimized CRISPR effector protein. An example of a codon optimized sequence, is in this instance a sequence optimized for expression in eukaryote, e.g., humans (e.g. being optimized for expression in humans), or for another eukaryote, animal or mammal as herein discussed; see, e.g., SaCas9 human codon optimized sequence in WO 2014/093622 (PCT/US2013/074667). It will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the composition and the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox (LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprises one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual HEPN domains.

In one aspect, the invention provides a method of modifying or editing a target transcript in a eukaryotic cell. In some embodiments, the method comprises allowing a CRISPR-Cas effector module complex to bind to the target polynucleotide to effect RNA base editing, wherein the CRISPR-Cas effector module complex comprises a Cas effector module complexed with a guide sequence hybridized to a target sequence within said target polynucleotide, wherein said guide sequence is linked to a direct repeat sequence. In some embodiments, the Cas effector module comprises a catalytically inactive CRISPR-Cas protein. In some embodiments, the guide sequence is designed to introduce one or more mismatches to the RNA/RNA duplex formed between the target sequence and the guide sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present application relates to modifying a target RNA sequence of interest (see, e.g., Cox et al., Science. 2017 Nov. 24; 358(6366):1019-1027). Using RNA-targeting rather than DNA targeting offers several advantages relevant for therapeutic development. First, there are substantial safety benefits to targeting RNA: there will be fewer off-target events because the available sequence space in the transcriptome is significantly smaller than the genome, and if an off-target event does occur, it will be transient and less likely to induce negative side effects. Second, RNA-targeting therapeutics will be more efficient because they are cell-type independent and not have to enter the nucleus, making them easier to deliver.

A further aspect of the present disclosure relates to the method and composition as envisaged herein for use in prophylactic or therapeutic treatment, preferably wherein said target locus of interest is within a human or animal and to methods of modifying an Adenine or Cytidine in a target RNA sequence of interest, comprising delivering to said target RNA, the composition as described herein. In particular embodiments, the CRISPR system and the adenosine deaminase, or catalytic domain thereof, are delivered as one or more polynucleotide molecules, as a ribonucleoprotein complex, optionally via particles, vesicles, or one or more viral vectors. In particular embodiments, the invention thus comprises compositions for use in therapy. This implies that the methods can be performed in vivo, ex vivo or in vitro. In particular embodiments, when the target is a human or animal target, the method is carried out ex vivo or in vitro.

A further aspect of the invention relates to the method as envisaged herein for use in prophylactic or therapeutic treatment, preferably wherein said target of interest is within a human or animal and to methods of modifying an Adenine or Cytidine in a target RNA sequence of interest, comprising delivering to said target RNA, the composition as described herein. In particular embodiments, the CRISPR system and the adenosine deaminase, or catalytic domain thereof, are delivered as one or more polynucleotide molecules, as a ribonucleoprotein complex, optionally via particles, vesicles, or one or more viral vectors.

In one aspect, the invention provides a method of generating a eukaryotic cell comprising a modified or edited gene. In some embodiments, the method comprises (a) introducing one or more vectors into a eukaryotic cell, wherein the one or more vectors drive expression of one or more of: Cas effector module, and a guide sequence linked to a direct repeat sequence, wherein the Cas effector module associate one or more effector domains that mediate base editing, and (b) allowing a CRISPR-Cas effector module complex to bind to a target polynucleotide to effect base editing of the target polynucleotide within said disease gene, wherein the CRISPR-Cas effector module complex comprises a Cas effector module complexed with the guide sequence that is hybridized to the target sequence within the target polynucleotide, wherein the guide sequence may be designed to introduce one or more mismatches between the RNA/RNA duplex formed between the guide sequence and the target sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

In particular embodiments, pre-complexed guide RNA and CRISPR effector protein, (optionally, adenosine deaminase fused to a CRISPR protein or an adaptor) are delivered as a ribonucleoprotein (RNP). RNPs have the advantage that they lead to rapid editing effects even more so than the RNA method because this process avoids the need for transcription. An important advantage is that both RNP delivery is transient, reducing off-target effects and toxicity issues. Efficient genome editing in different cell types has been observed by Kim et al. (2014, Genome Res. 24(6):1012-9), Paix et al. (2015, Genetics 204(1):47-54), Chu et al. (2016, BMC Biotechnol. 16:4), and Wang et al. (2013, Cell. 9; 153(4):910-8).

In particular embodiments, the ribonucleoprotein is delivered by way of a polypeptide-based shuttle agent as described in WO2016161516. WO2016161516 describes efficient transduction of polypeptide cargos using synthetic peptides comprising an endosome leakage domain (ELD) operably linked to a cell penetrating domain (CPD), to a histidine-rich domain and a CPD. Similarly these polypeptides can be used for the delivery of CRISPR-effector based RNPs in eukaryotic cells.

The Cas proteins may include homologues and orthologues of the Cas proteins described herein. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of Homologous proteins may but need not be structurally related, or are only partially structurally related. An “orthologue” of a protein as used herein is a protein of a different species which performs the same or a similar function as the protein it is an orthologue of Orthologous proteins may but need not be structurally related, or are only partially structurally related. Thus, when reference is made to mouse genes and proteins, it is understood that the same is believed to apply to the corresponding ortholog in humans or other species.

Likewise, when referencing a Cas protein, it is understood to likewise apply to orthologs and homologs.

The CRISPR-CRISPR associated (Cas) systems of bacterial and archaeal adaptive immunity are some such systems that show extreme diversity of protein composition and genomic loci architecture. The CRISPR-Cas system loci has more than 50 gene families and there is no strictly universal genes indicating fast evolution and extreme diversity of loci architecture. So far, adopting a multi-pronged approach, there is comprehensive cas gene identification of about 395 profiles for 93 Cas proteins. Classification includes signature gene profiles plus signatures of locus architecture. A new classification of CRISPR-Cas systems is proposed in which these systems are broadly divided into two classes, Class 1 with multisubunit effector complexes and Class 2 with single-subunit effector modules exemplified by the Cas9 protein. Novel effector proteins associated with Class 2 CRISPR-Cas systems may be developed as powerful genome engineering tools and the prediction of putative novel effector proteins and their engineering and optimization is important.

The effector protein may comprise a chimeric effector protein comprising a first fragment from a first effector protein ortholog and a second fragment from a second effector protein ortholog, and wherein the first and second effector protein orthologs are different.

### Guide Molecules

The nucleotide sequences may be guide molecules or comprise coding sequences of guide molecules (or interchangeably referred to as guide sequences or guides). In some cases, a guide molecule is a guide RNA. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

The guide sequence-encoding sequences and/or Cas protein-encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In some embodiments, it is of interest to reduce the susceptibility of the guide molecule to RNA cleavage, such as to cleavage by a Cas protein. Accordingly, in particular embodiments, the guide molecule is adjusted to avoid cleavage by a Cas protein or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′phosphorothioate (MS), S-constrained ethyl (cEt), or 2′-O-methyl 3′thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., Med Chem Comm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucleotides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl (cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (me1Ψ), 5-methoxyuridine (5moU), inosine, 7-methylguanosine, 2′-O-methyl 3′phosphorothioate (MS), S-constrained ethyl (cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sulfonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of the guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas protein (Chen et al. Cell. (2013); 155(7): 1479-1491). In particular embodiments the stem of the stemloop is extended by at least 1, 2, 3, 4, 5 or more complementary basepairs (i.e. corresponding to the addition of 2, 4, 6, 8, 10 or more nucleotides in the guide molecule). In particular embodiments these are located at the end of the stem, adjacent to the loop of the stemloop.

In particular embodiments, the susceptibility of the guide molecule to RNases or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment, the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the complementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the CRISPR-Cas system or complex function. The invention can involve applying the chemical source or energy so as to have the guide function and the CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans; 4/164/rs2), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/journal/v8/n5/full/nchembio.922.html).

A chemical inducible system can be an estrogen receptor (ER) based system inducible by 4-hydroxytamoxifen (40HT) (see, e.g., www.pnas.org/content/104/3/1027.abstract). A mutated ligand-binding domain of the estrogen receptor called ERT2 translocate into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas13 CRISPR-Cas complex or system, and the binding will induce the change of sub-cellular localization of the polypeptide, leading to the entire polypeptide entering the nucleus of cells. Once inside the nucleus, the guide protein and the other components of the Cas13 CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc., as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100 .mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1 V/cm and 20 V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

A further aspect relates to an isolated cell obtained or obtainable from the methods described herein comprising the composition described herein or progeny of said modified cell, preferably wherein said cell comprises a hypoxanthine or a guanine in replace of said Adenine in said target RNA of interest compared to a corresponding cell not subjected to the method. In particular embodiments, the cell is a eukaryotic cell, preferably a human or non-human animal cell, optionally a therapeutic T cell or an antibody-producing B-cell.

In some embodiments, the modified cell is a therapeutic T cell, such as a T cell suitable for adoptive cell transfer therapies (e.g., CAR-T therapies). The modification may result in one or more desirable traits in the therapeutic T cell, as described further herein.

The invention further relates to a method for cell therapy, comprising administering to a patient in need thereof the modified cell described herein, wherein the presence of the modified cell remedies a disease in the patient.

The present invention may be further illustrated and extended based on aspects of CRISPR-Cas development and use as set forth in the following articles and particularly as relates to delivery of a CRISPR protein complex and uses of an RNA guided endonuclease in cells and organisms:
Multiplex genome engineering using CRISPR-Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR-Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi: 10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013);
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR-Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR-Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/nature15521. Epub 2015 Sep. 16.
Cpf1 Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1.
Gao et al, “Engineered Cpf1 Enzymes with Altered PAM Specificities,” bioRxiv 091611; doi: http://dx.doi.org/10.1101/091611 (Dec. 4, 2016).
Cox et al., “RNA editing with CRISPR-Cas13,” Science. 2017 Nov. 24; 358(6366):1019-1027. doi: 10.1126/science.aaq0180. Epub 2017 Oct. 25.

each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and guide RNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR-Cas9 knockout and nucleotide preference at the cleavage site. The authors also found that the sequence preference for CRISPRi/a is substantially different from that for CRISPR-Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Canver et al. (2015) demonstrated a CRISPR-Cas9-based functional investigation of non-coding genomic elements. The authors we developed pooled CRISPR-Cas9 guide RNA libraries to perform in situ saturating mutagenesis of the human and mouse BCL11A enhancers which revealed critical features of the enhancers.
Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.
Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2c1 depends on both crRNA and tracrRNA for DNA cleavage. The third enzyme (C2c2) contains two predicted HEPN RNase domains and is tracrRNA independent.
Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.
Cox et al., (2017) reported the use of catalytically inactive Cas13 (dCas13) to direct adenosine-to-inosine deaminase activity by ADAR2 (adenosine deaminase acting on RNA type 2) to transcripts in mammalian cells. The system, referred to as RNA Editing for Programmable A to I Replacement (REPAIR), has no strict sequence constraints and can be used to edit full-length transcripts. The authors further engineered the system to create a high-specificity variant and minimized the system to facilitate viral delivery.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12 Feb. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

### Base Editing

The modulating agents herein may comprise a Cas protein or a variant thereof (e.g., inactive or dead Cas) fused with a functional domains. The Cas protein may be a dead Cas protein or a Cas nickase protein. For example, the compositions herein may comprise one or more components of a base editing system. In some embodiments, compositions herein comprise nucleotide sequence comprising encoding sequences for one or more components of a base editing system. A base-editing system may comprise a deaminase (e.g., an adenosine deaminase or cytidine deaminase) fused with a Cas protein or a variant thereof. In certain examples, the system comprises a mutated form of an adenosine deaminase fused with a dead CRISPR-Cas or CRISPR-Cas nickase. The mutated form of the adenosine deaminase may have both adenosine deaminase and cytidine deaminase activities. Examples of base editing systems include those described in WO2019071048, WO2019084063, WO2019126716, WO2019126709, WO2019126762, WO2019126774, Cox DBT, et al., RNA editing with CRISPR-Cas13, Science. 2017 Nov. 24; 358(6366):1019-1027; Abudayyeh O O, et al., A cytosine deaminase for programmable single-base RNA editing, Science 26 Jul. 2019: Vol. 365, Issue 6451, pp. 382-386; Gaudelli N M et al., Programmable base editing of A⋅T to G⋅C in genomic DNA without DNA cleavage, Nature volume 551, pages 464-471 (23 Nov. 2017); Komor A C, et al., Programmable editing of a target base in genomic DNA without double-stranded DNA cleavage. Nature. 2016 May 19; 533(7603):420-4.

### TALE Systems

The composition may comprise one or more components of a TALE system. The composition may also comprise nucleotide sequences that are or encode one or more components of a TALE system. As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In some embodiments, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

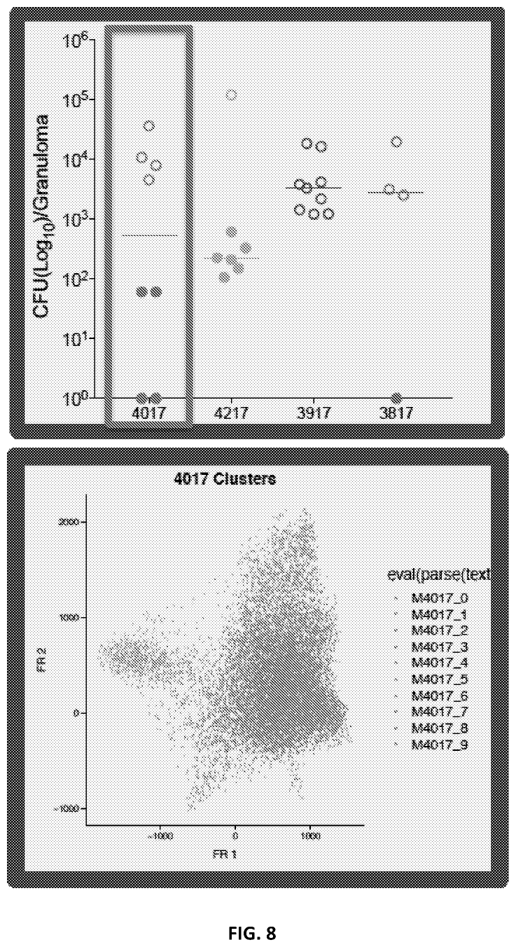

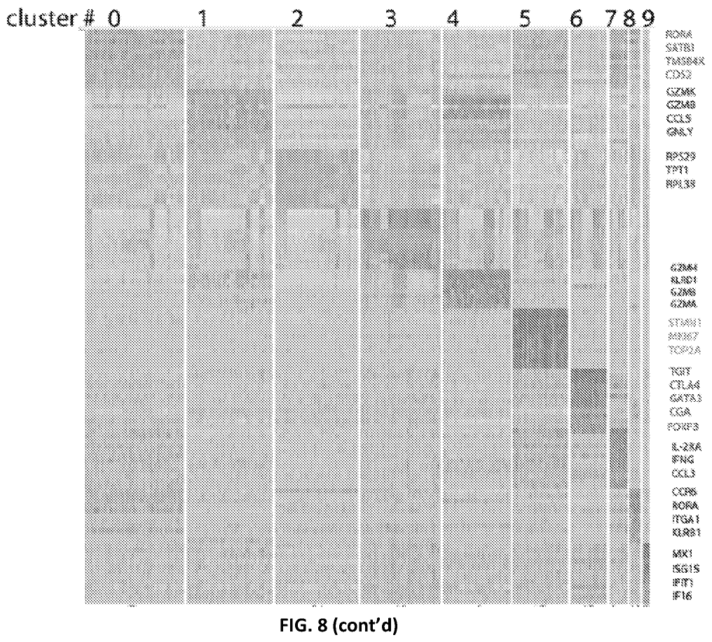

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

M D P I R S R T P S P A R E L L S G P Q P D G V
Q P T A D R G V S P P A G G P L D G L P A R R T
M S R T R L P S P P A P S P A F S A D S F S D L
L R Q F D P S L F N T S L F D S L P P F G A H H
T E A A T G E W D E V Q S G L R A A D A P P P T
M R V A V T A A R P P R A K P A P R R R A A Q P
S D A S P A A Q V D L R T L G Y S Q Q Q Q E K I
K P K V R S T V A Q H H E A L V G H G F T H A H
I V A L S Q H P A A L G T V A V K Y Q D M I A A
L P E A T H E A I V G V G K Q W S G A R A L E A
L L T V A G E L R G P P L Q L D T G Q L L K I A
K R G G V T A V E A V H A W R N A L T G A P L N

An exemplary amino acid sequence of a C-terminal capping region is:

R P A L E S I V A Q L S R P D P A L A A L T N D
H L V A L A C L G G R P A L D A V K K G L P H A
P A L I K R T N R R I P E R T S H R V A D H A Q
V V R V L G F F Q C H S H P A Q A F D D A M T Q
F G M S R H G L L Q L F R R V G V T E L E A R S
G T L P P A S Q R W D R I L Q A S G M K R A K P
S P T S T Q T P D Q A S L H A F A D S L E R D L
D A P S P M H E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### Zn-Finger Nucleases

The composition may comprise one or more Zn-finger nucleases or nucleic acids encoding thereof. In some cases, the nucleotide sequences may comprise coding sequences for Zn-Finger nucleases. Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

The composition may comprise one or more meganucleases or nucleic acids encoding thereof. As disclosed herein editing can be made by way of meganucleases, which are endodeoxyribonucleases characterized by a large recognition site (double-stranded DNA sequences of 12 to 40 base pairs). In some cases, the nucleotide sequences may comprise coding sequences for meganucleases. Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, any of the nucleases, including the modified nucleases as described herein, may be used in the methods, compositions, and kits according to the invention. In particular embodiments, nuclease activity of an unmodified nuclease may be compared with nuclease activity of any of the modified nucleases as described herein, e.g. to compare for instance off-target or on-target effects. Alternatively, nuclease activity (or a modified activity as described herein) of different modified nucleases may be compared, e.g. to compare for instance off-target or on-target effects.

### RNAi

In some embodiments, the genetic modulating agents may be interfering RNAs. In some cases, the nucleotide sequence may comprise coding sequence for one or more interfering RNAs. In certain examples, the nucleotide sequence may be interfering RNA (RNAi). As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

In certain embodiments, a modulating agent may comprise silencing one or more endogenous genes. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Other Examples of Genetic Modulating Agents

The composition may comprise one or more other types of genetic modulating agents. The nucleotide sequences may be or comprise encoding sequences of the one or more genetic modulating agents. Agents useful in the methods as disclosed herein are proteins and/or peptides or fragment thereof, which inhibit the gene expression of a target gene or gene product, or the function of a target protein. Such agents include, for example but are not limited to protein variants, mutated proteins, therapeutic proteins, truncated proteins and protein fragments. Protein agents can also be selected from a group comprising mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. As disclosed herein, a protein which inhibits the function of a target protein may be a soluble dominant negative form of the target protein or a functional fragment or variant thereof which inhibits wild-type full length target protein function.

In certain embodiments, the agents may be small molecules, antibodies, therapeutic antibody, antibody fragment, antibody-like protein scaffold, aptamer, protein, genetic modifying agent or small molecule. The chemical entity or biological product is preferably, but not necessarily a low molecular weight compound, but may also be a larger compound, or any organic or inorganic molecule effective in the given situation, including modified and unmodified nucleic acids such as antisense nucleic acids, RNAi, such as siRNA or shRNA, CRISPR-Cas systems, peptides, peptidomimetics, receptors, ligands, and antibodies, aptamers, polypeptides, nucleic acid analogues or variants thereof. Examples include an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. Agents can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), modified RNA (mod-RNA), single guide RNA etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides, CRISPR guide RNA, for example that target a CRISPR enzyme to a specific DNA target sequence etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, minibodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a gene within the cell. In some embodiments, the agent is any chemical, entity or moiety, including without limitation synthetic and naturally-occurring non-proteinaceous entities. In certain embodiments the agent is a small molecule having a chemical moiety. Agents can be known to have a desired activity and/or property, or can be selected from a library of diverse compounds.

### Exogenous Genes

In some embodiments, the modulating agents may be exogenous genes or functional fragments thereof. When delivered to specific nuclei in multinucleated cells, the exogenous gene may express a product (e.g., protein or nucleic acid) that manipulates the function of the cells or treats a disease related to the function of the cells. For example, the exogenous gene may encode dystrophin or a functional fragment thereof.

### Hormones, Cytokines, Growth Factors

In certain embodiments, an agent may be a hormone, a cytokine, a lymphokine, a growth factor, a chemokine, a cell surface receptor ligand such as a cell surface receptor agonist or antagonist, or a mitogen.

Non-limiting examples of hormones include growth hormone (GH), adrenocorticotropic hormone (ACTH), dehydroepiandrosterone (DHEA), cortisol, epinephrine, thyroid hormone, estrogen, progesterone, testosterone, or combinations thereof.

Non-limiting examples of cytokines include lymphokines (e.g., interferon-γ, IL-2, IL-3, IL-4, IL-6, granulocyte-macrophage colony-stimulating factor (GM-CSF), interferon-γ, leukocyte migration inhibitory factors (T-LIF, B-LIF), lymphotoxin-alpha, macrophage-activating factor (MAF), macrophage migration-inhibitory factor (MIF), neuroleukin, immunologic suppressor factors, transfer factors, or combinations thereof), monokines (e.g., IL-1, TNF-alpha, interferon-a, interferon-β, colony stimulating factors, e.g., CSF2, CSF3, macrophage CSF or GM-CSF, or combinations thereof), chemokines (e.g., beta-thromboglobulin, C chemokines, CC chemokines, CXC chemokines, CX3C chemokines, macrophage inflammatory protein (MIP), or combinations thereof), interleukins (e.g., IL-1, IL-2, IL-3, IL-4, IL-5, IL-6, IL-7, IL-8, IL-9, IL-10, IL-11, IL-12, IL-13, IL-14, IL-15, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or combinations thereof), and several related signaling molecules, such as tumour necrosis factor (TNF) and interferons (e.g., interferon-a, interferon-β, interferon-γ, interferon-λ, or combinations thereof).

Non-limiting examples of growth factors include those of fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, glucocorticoids, or combinations thereof.

Non-limiting examples of mitogens include phytohaemagglutinin (PHA), concanavalin A (conA), lipopolysaccharide (LPS), pokeweed mitogen (PWM), phorbol ester such as phorbol myristate acetate (PMA) with or without ionomycin, or combinations thereof.

Non-limiting examples of cell surface receptors the ligands of which may act as agents include Toll-like receptors (TLRs) (e.g., TLR1, TLR2, TLR3, TLR4, TLR5, TLR6, TLR7, TLR8, TLR9, TLR10, TLR11, TLR12 or TLR13), CD80, CD86, CD40, CCR7, or C-type lectin receptors.

### Pharmaceutical Compositions

Pharmaceutical compositions or vaccines are also contemplated within the scope of the disclosure. In some cases, the one or more modulating agents may be comprised in a pharmaceutical composition or formulation, or a vaccine. One aspect of the invention provides for a composition, pharmaceutical composition or vaccine directed to MTB infected cells.

A “pharmaceutical composition” refers to a composition that usually contains an excipient, such as a pharmaceutically acceptable carrier that is conventional in the art and that is suitable for administration to cells or to a subject. Pharmaceutically acceptable as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8(2000), Wang W. “Lyophilization and development of solid protein pharmaceuticals.” Int. J. Pharm. 203(1-2):1-60(2000), Charman, W N “Lipids, lipophilic drugs, and oral drug delivery-some emerging concepts.” J Pharm Sci. 89(8):967-78(2000), Powell et al. “Compendium of excipients for parenteral formulations” PDAJ Pharm Sci Technol. 52:238-311(1998) and the citations therein for additional information related to formulations, excipients and carriers well known to pharmaceutical chemists.

The medicaments are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

The modulating agents disclosed herein (e.g., antibodies) may be used in a pharmaceutical composition when combined with a pharmaceutically acceptable carrier. Such compositions comprise a therapeutically-effective amount of the agent and a pharmaceutically acceptable carrier. Such a composition may also further comprise (in addition to an agent and a carrier) diluents, fillers, salts, buffers, stabilizers, solubilizers, and other materials well known in the art. Compositions comprising the agent can be administered in the form of salts provided the salts are pharmaceutically acceptable. Salts may be prepared using standard procedures known to those skilled in the art of synthetic organic chemistry.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or prodrug formulations. It will be understood that, as used herein, references to specific agents (e.g., neuromedin U receptor agonists or antagonists), also include the pharmaceutically acceptable salts thereof.

Methods of administrating the pharmacological compositions, including agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor or infected tissue), thus requiring only a fraction of the systemic dose. See, for example, Goodson, In: Medical Applications of Controlled Release, 1984. (CRC Press, Boca Raton, Fla.).

The amount of the agents which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. In general, the daily dose range of a drug lie within the range known in the art for a particular drug or biologic. Effective doses may be extrapolated from dose-response curves derived from in vitro or animal model test systems. Ultimately the attending physician will decide on the appropriate duration of therapy using compositions of the present invention. Dosage will also vary according to the age, weight and response of the individual patient.

Methods for administering antibodies for therapeutic use is well known to one skilled in the art. In certain embodiments, small particle aerosols of antibodies or fragments thereof may be administered (see e.g., Piazza et al., J. Infect. Dis., Vol. 166, pp. 1422-1424, 1992; and Brown, Aerosol Science and Technology, Vol. 24, pp. 45-56, 1996). In certain embodiments, antibodies are administered in metered-dose propellant driven aerosols. In certain embodiments, antibodies may be administered in liposomes, i.e., immunoliposomes (see, e.g., Maruyama et al., Biochim. Biophys. Acta, Vol. 1234, pp. 74-80, 1995). In certain embodiments, immunoconjugates, immunoliposomes or immunomicrospheres containing an agent of the present invention is administered by inhalation.

In certain embodiments, antibodies may be topically administered to mucosa, such as the oropharynx, nasal cavity, respiratory tract, gastrointestinal tract, eye such as the conjunctival mucosa, vagina, urogenital mucosa, or for dermal application. In certain embodiments, antibodies are administered to the nasal, bronchial or pulmonary mucosa. In order to obtain optimal delivery of the antibodies to the pulmonary cavity in particular, it may be advantageous to add a surfactant such as a phosphoglyceride, e.g. phosphatidylcholine, and/or a hydrophilic or hydrophobic complex of a positively or negatively charged excipient and a charged antibody of the opposite charge.

Other excipients suitable for pharmaceutical compositions intended for delivery of antibodies to the respiratory tract mucosa may be a) carbohydrates, e.g., monosaccharides such as fructose, galactose, glucose. D-mannose, sorbiose, and the like; disaccharides, such as lactose, trehalose, cellobiose, and the like; cyclodextrins, such as 2-hydroxypropyl-β-cyclodextrin; and polysaccharides, such as raffinose, maltodextrins, dextrans, and the like; b) amino acids, such as glycine, arginine, aspartic acid, glutamic acid, cysteine, lysine and the like; c) organic salts prepared from organic acids and bases, such as sodium citrate, sodium ascorbate, magnesium gluconate, sodium gluconate, tromethamine hydrochloride, and the like: d) peptides and proteins, such as aspartame, human serum albumin, gelatin, and the like; e) alditols, such mannitol, xylitol, and the like, and f) polycationic polymers, such as chitosan or a chitosan salt or derivative.

For dermal application, the antibodies of the present invention may suitably be formulated with one or more of the following excipients: solvents, buffering agents, preservatives, humectants, chelating agents, antioxidants, stabilizers, emulsifying agents, suspending agents, gel-forming agents, ointment bases, penetration enhancers, and skin protective agents.

Examples of solvents are e.g. water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof.

Examples of buffering agents are e.g. citric acid, acetic acid, tartaric acid, lactic acid, hydrogenphosphoric acid, diethyl amine etc. Suitable examples of preservatives for use in compositions are parabenes, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Examples of humectants are glycerin, propylene glycol, sorbitol, lactic acid, urea, and mixtures thereof.

Examples of antioxidants are butylated hydroxy anisole (BHA), ascorbic acid and derivatives thereof, tocopherol and derivatives thereof, cysteine, and mixtures thereof.

Examples of emulsifying agents are naturally occurring gums, e.g. gum acacia or gum tragacanth; naturally occurring phosphatides, e.g. soybean lecithin, sorbitan monooleate derivatives: wool fats; wool alcohols; sorbitan esters; monoglycerides; fatty alcohols; fatty acid esters (e.g. triglycerides of fatty acids); and mixtures thereof.

Examples of suspending agents are e.g. celluloses and cellulose derivatives such as, e.g., carboxymethyl cellulose, hydroxyethylcellulose, hydroxypropylcellulose, hydroxypropylmethylcellulose, carraghenan, acacia gum, arabic gum, tragacanth, and mixtures thereof.

Examples of gel bases, viscosity-increasing agents or components which are able to take up exudate from a wound are: liquid paraffin, polyethylene, fatty oils, colloidal silica or aluminum, zinc soaps, glycerol, propylene glycol, tragacanth, carboxyvinyl polymers, magnesium-aluminum silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans, hyaluronates (e.g. hyaluronate gel optionally containing sodium chloride), and alginates including propylene glycol alginate.

Examples of ointment bases are e.g. beeswax, paraffin, cetanol, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

Examples of hydrophobic or water-emulsifying ointment bases are paraffins, vegetable oils, animal fats, synthetic glycerides, waxes, lanolin, and liquid polyalkylsiloxanes. Examples of hydrophilic ointment bases are solid macrogols (polyethylene glycols). Other examples of ointment bases are triethanolamine soaps, sulphated fatty alcohol and polysorbates.

Examples of other excipients are polymers such as carmelose, sodium carmelose, hydroxypropylmethylcellulose, hydroxyethylcellulose, hydroxypropylcellulose, pectin, xanthan gum, locust bean gum, acacia gum, gelatin, carbomer, emulsifiers like vitamin E, glyceryl stearates, cetanyl glucoside, collagen, carrageenan, hyaluronates and alginates and chitosans.

The dose of antibody required in humans to be effective in the treatment of TB infection differs with the type and severity of the TB to be treated, the age and condition of the patient, etc. Typical doses of antibody to be administered are in the range of 1 μg to 1 g, preferably 1-1000 μg, more preferably 2-500, even more preferably 5-50, most preferably 10-20 g per unit dosage form. In certain embodiments, infusion of antibodies of the present invention may range from 10-500 mg/m².

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transduction with viral (typically lentivirus, adeno associated virus (AAV) and adenovirus) vectors.

In certain embodiments, an agent that reduces a gene signature as described herein is used to treat a subject in need thereof having a TB infection.

The pharmaceutical composition can be applied parenterally, rectally, orally or topically. Preferably, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infuse. The skilled person will understand that compositions which are to be administered orally or topically will usually not comprise cells, although it may be envisioned for oral compositions to also comprise cells, for example when gastro-intestinal tract indications are treated. Each of the cells or active components (e.g., modulants, immunomodulants, antigens) as discussed herein may be administered by the same route or may be administered by a different route. By means of example, and without limitation, cells may be administered parenterally and other active components may be administered orally.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propyleneglycol or polyethylene glycol may be included. The composition may include one or more cell protective molecules, cell regenerative molecules, growth factors, anti-apoptotic factors or factors that regulate gene expression in the cells. Such substances may render the cells independent of their environment. Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

If desired, cell preparation can be administered on a support, scaffold, matrix or material to provide improved tissue regeneration. For example, the material can be a granular ceramic, or a biopolymer such as gelatine, collagen, or fibrinogen. Porous matrices can be synthesized according to standard techniques (e.g., Mikos et al., Biomaterials 14: 323, 1993; Mikos et al., Polymer 35:1068, 1994; Cook et al., J. Biomed. Mater. Res. 35:513, 1997). Such support, scaffold, matrix or material may be biodegradable or non-biodegradable. Hence, the cells may be transferred to and/or cultured on suitable substrate, such as porous or non-porous substrate, to provide for implants.

For example, cells that have proliferated, or that are being differentiated in culture dishes, can be transferred onto three-dimensional solid supports in order to cause them to multiply and/or continue the differentiation process by incubating the solid support in a liquid nutrient medium of the invention, if necessary. Cells can be transferred onto a three-dimensional solid support, e.g. by impregnating the support with a liquid suspension containing the cells. The impregnated supports obtained in this way can be implanted in a human subject. Such impregnated supports can also be re-cultured by immersing them in a liquid culture medium, prior to being finally implanted. The three-dimensional solid support needs to be biocompatible so as to enable it to be implanted in a human. It may be biodegradable or non-biodegradable.

The cells or cell populations can be administered in a manner that permits them to survive, grow, propagate and/or differentiate towards desired cell types (e.g. differentiation) or cell states. The cells or cell populations may be grafted to or may migrate to and engraft within the intended organ. In certain embodiments, a pharmaceutical cell preparation as taught herein may be administered in a form of liquid composition. In embodiments, the cells or pharmaceutical composition comprising such can be administered systemically, topically, within an organ or at a site of organ dysfunction or lesion.

The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

A further aspect of the invention provides a modulating infection in a population of infected cells as taught herein. The terms “cell population” or “population” denote a set of cells having characteristics in common. The characteristics may include in particular the one or more marker(s) or gene or gene product signature(s) as taught herein. The cells as taught herein may be comprised in a cell population. By means of example, the specified cells may constitute at least 40% (by number) of all cells of the cell population, for example, at least 45%, preferably at least 50%, at least 55%, more preferably at least 60%, at least 65%, still more preferably at least 70%, at least 75%, even more preferably at least 80%, at least 85%, and yet more preferably at least 90%, at least 95%, at least 96%, at least 97%, at least 98%, at least 99%, or even 100% of all cells of the cell population.

The isolated cells, cells, or populations thereof as disclosed throughout this specification may be suitably cultured or cultivated in vitro. The term “in vitro” generally denotes outside, or external to, a body, e.g., an animal or human body. The term encompasses “ex vivo”. The terms “culturing” or “cell culture” are common in the art and broadly refer to maintenance of cells and potentially expansion (proliferation, propagation) of cells in vitro. Typically, animal cells, such as mammalian cells, such as human cells, are cultured by exposing them to (i.e., contacting them with) a suitable cell culture medium in a vessel or container adequate for the purpose (e.g., a 96-, 24-, or 6-well plate, a T-25, T-75, T-150 or T-225 flask, or a cell factory), at art-known conditions conducive to in vitro cell culture, such as temperature of 37° C., 5% v/v CO2 and >95% humidity. The term “medium” as used herein broadly encompasses any cell culture medium conducive to maintenance of cells, preferably conducive to proliferation of cells. Typically, the medium will be a liquid culture medium, which facilitates easy manipulation (e.g., decantation, pipetting, centrifugation, filtration, and such) thereof

### Adoptive Cell Transfer

In certain example embodiments, the methods and compositions herein may be used for adoptive cell transfer. For example, a cells, e.g., macrophage or macrophage population, mast cells, and/or Th1-Th17 cells, may be isolated from the subject and modulated as described above and delivered back to the subject.

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAPi (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190 KD bcr-abl); Pml/RARa (promyelocytic leukaemia/retinoic acid receptor a); PRANE (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (Dl), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD 11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3):

IEVMYPPPYLDNEKSNGTIIHVKGKHLCPSPLFPGPSKPFWVLVVVG
GVLACYSLLVTVAFIIFWVRSKRSRLLHSDYMNMTPRRPGPTRKHYQ
PYAPPRDFAAYRS)) (SEQ ID NO: 1).

Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ ID NO:2) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY and continuing all the way to the carboxy-terminus of the protein. The sequence is reproduced herein:

(SEQ ID NO: 1)
IEVMYPPPYLDNEKSNGTIIHVKGKHLCPSPLFPGPSKPFWVLVVVG
GVLACYSLLVTVAFIIFWVRSKRSRLLHSDYMNMTPRRPGPTRKHYQ
PYAPPRDFAAYRS.

Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signalling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signalling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+ Th1 cells and CD8+ CTLs to induce a synergistic antitumour response (see, e.g., Li et al., Adoptive cell therapy with CD4+ T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4⁺ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, 3-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, 3-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14⁺ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of 125I labeled β2-microglobulin (β2m) into MHC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

### Differentiating Macrophage Subpopulations

In certain embodiments, a method for differentiating one or more macrophage subpopulations infected by Mtb from one or more uninfected macrophage subpopulations may comprise assaying the macrophage or macrophage population for the presence, or overexpression compared to wild type macrophages of at least one of cytokine receptors, SLAM family members, and kinases and/or at least differentiation of macrophage state, wherein identification of the presence or overexpression indicates an infected macrophage. Example cytokine receptors may include IFNGR1, and ILRN. Example SLAM family members may include SLAM 7, SLAM5. Example kinases may include HCK, and CAMK1. Example differentiators of macrophage state may include M1, M2, HLA-DRB1 and CD86. The method may further comprise, or alternatively comprise, assaying the macrophage or population of macrophages for the presence, or overexpression compared to wild type macrophages of at least one of ApoE, CD36, CD52, and IL8 ApoE, CD36, CD52, and/or IL8, wherein detection of the present or overexpression indicates an uninfected macrophage population. Accordingly, the method may further comprise separating the infected and uninfected sub-populations. Separating the macrophage may comprise labelling or tagging one of the infected or the uninfected subpopulations or by differentially labelling or tagging the infected and the uninfected subpopulations. The method may further comprise modulating the infected or uninfected macrophage with one of the modulators described above to promote a control phenotype.

### Model Cell Lines

In certain aspects, embodiments disclosed herein are directed to model macrophage cell lines. The macrophage cell line may be isolated using the method described above. In certain example embodiments, the macrophage may be a CD14+ macrophage. In certain other example embodiments the CD14+ macrophage may have at least one of the following genes regulated: CD206, CD86, and CD32; and/or at lease CD163 down-regulated. In certain example embodiments, the model cell line may be derived from a primary human CD14+ cell line.

### Method of Detecting

The methods of diagnosing comprise a step of detecting a gene expression profile in one or more cells or tissue associated with Mycobacterium tuberculosis infection. The step of detecting can, in one embodiment, comprise whether one or more genes is overexpressed or underexpressed compared to a cell that is not infected. In some preferred embodiments, the cells are immune cells, in some particular embodiments, the cells are macrophages.

In one embodiment, the signature genes may be detected by immunofluorescence, immunohistochemistry, fluorescence activated cell sorting (FACS), mass cytometry (CyTOF), Drop-seq, RNA-seq, scRNA-seq, InDrop, single cell qPCR, MERFISH (multiplex (in situ) RNA FISH) and/or by in situ hybridization. Other methods including absorbance assays and colorimetric assays are known in the art and may be used herein.

All gene name symbols provided herein refer to the gene as commonly known in the art. The examples described herein refer to the human gene names and it is to be understood that the present invention also encompasses genes from other organisms (e.g., mouse genes). Gene symbols may be those referred to by the HUGO Gene Nomenclature Committee (HGNC) or National Center for Biotechnology Information (NCBI). Any reference to the gene symbol is a reference made to the entire gene or variants of the gene. The signature as described herein may encompass any of the genes described herein. In certain embodiments, the gene signature includes surface expressed and secreted proteins. Not being bound by a theory, surface proteins may be targeted for detection and isolation of cell types, or may be targeted therapeutically to modulate an immune response.

In certain embodiments, the gene signature is detected in a bulk sample, whereby the gene signature is detected by deconvolution of bulk expression data such that gene expression is assigned to infected cells and non-infected cells in the sample. In certain embodiments, detecting the gene signature comprises detecting downregulation of the down signature and/or upregulation of the up signature, and wherein not detecting the gene signature comprises detecting upregulation of the down signature and/or downregulation of the up signature.

The step of detecting can, in one embodiment, comprise whether one or more genes is underexpressed compared to a cell that is not infected.

The step of detecting can include detecting whether one or more genes is overexpressed and whether one or more genes is underexpressed in a cell or tissue in a subject comprising a Mycobacterium tuberculosis infection as compared to a cell that is not infected.

In one embodiment, the method comprises detecting or quantifying MTB infected cells in a biological sample. A marker, for example, a gene or gene product, for example a peptide, polypeptide, protein, or nucleic acid, or a group of two or more markers, is detected or measured qualitatively or quantitatively in a tested object (e.g., in or on a cell, cell population, tissue, organ, or organism, e.g., in a biological sample of a subject) when the presence or absence and/or quantity of said marker or said group of markers is detected or determined in the tested object, preferably substantially to the exclusion of other molecules and analytes, e.g., other genes or gene products.

In one particular embodiment, signature genes and biomarkers related to MTB infection and TB symptoms may be identified by comparing single cell expression profiles obtained from uninfected cells and cells infected with detectable copies of MTB, such as MTB strain expressing fluorescence markers.

Various aspects and embodiments of the invention may involve analyzing gene signatures, protein signature, and/or other genetic or epigenetic signature based on single cell analyses (e.g. single cell RNA sequencing) or alternatively based on cell population analyses, as is defined herein elsewhere.

In one embodiment, the method comprises detecting or quantifying MTB infected cells in a biological sample. A marker, for example a gene or gene product, for example a peptide, polypeptide, protein, or nucleic acid, or a group of two or more markers, is “detected” or “measured” in a tested object (e.g., in or on a cell, cell population, tissue, organ, or organism, e.g., in a biological sample of a subject) when the presence or absence and/or quantity of said marker or said group of markers is detected or determined in the tested object, preferably substantially to the exclusion of other molecules and analytes, e.g., other genes or gene products.

In one embodiment, the method comprises detecting or quantifying a sub-population of cells harboring persistent or latent infection in a biological sample. A marker, for example a gene or gene product, for example a peptide, polypeptide, protein, or nucleic acid, or a group of two or more markers, is detected or measured in a tested object (e.g., in or on a cell, cell population, tissue, organ, or organism, e.g., in a biological sample of a subject) when the presence or absence and/or quantity of said marker or said group of markers is detected or determined in the tested object, preferably substantially to the exclusion of other molecules and analytes, e.g., other genes or gene products.

In one embodiment, the method comprises detecting or quantifying MTB infected cells in a biological sample. A marker, for example a gene or gene product, for example a peptide, polypeptide, protein, or nucleic acid, or a group of two or more markers, is “detected” or “measured” in a tested object (e.g., in or on a cell, cell population, tissue, organ, or organism, e.g., in a biological sample of a subject) when the presence or absence and/or quantity of said marker or said group of markers is detected or determined in the tested object, preferably substantially to the exclusion of other molecules and analytes, e.g., other genes or gene products.

In one embodiment, the method comprises detecting or quantifying MTB infection state or MTB copy numbers in TB cells in a biological sample. A marker, for example a gene or gene product, for example a peptide, polypeptide, protein, or nucleic acid, or a group of two or more markers, is “detected” or “measured” in a tested object (e.g., in or on a cell, cell population, tissue, organ, or organism, e.g., in a biological sample of a subject) when the presence or absence and/or quantity of said marker or said group of markers is detected or determined in the tested object, preferably substantially to the exclusion of other molecules and analytes, e.g., other genes or gene products.

In some embodiments, overexpression of a gene associated with a pathway is provided, in some instances, the pathway can be selected from p53-pathwy, NFkB pathway or vitamin D receptor pathway.

In a preferred embodiment, the method comprises detecting or quantifying pathogen in an easily obtainable sample such as blood or body fluid as a proxy or surrogate indicative of infection states of the tested subpopulation of cells, a different subpopulation of cells, a different tissue, or the whole organism. Particularly preferred cells are immune cells, more particularly macrophages.

In certain embodiments, the cell types disclosed herein may be detected, quantified or isolated using a technique selected from the group consisting of flow cytometry, mass cytometry, fluorescence activated cell sorting (FACS), fluorescence microscopy, affinity separation, magnetic cell separation, microfluidic separation, RNA-seq (e.g., bulk or single cell), quantitative PCR, MERFISH (multiplex (in situ) RNA FISH) and combinations thereof. The technique may employ one or more agents capable of specifically binding to one or more gene products expressed or not expressed by the cells, preferably on the cell surface of the cells. The one or more agents may be one or more antibodies. Other methods including absorbance assays and colorimetric assays are known in the art and may be used herein.

Depending on factors that can be evaluated and decided on by a skilled person, such as, inter alia, the type of a marker (e.g., peptide, polypeptide, protein, or nucleic acid), the type of the tested object (e.g., a cell, cell population, tissue, organ, or organism, e.g., the type of biological sample of a subject, e.g., whole blood, plasma, serum, tissue biopsy), the expected abundance of the marker in the tested object, the type, robustness, sensitivity and/or specificity of the detection method used to detect the marker, etc., the marker may be measured directly in the tested object, or the tested object may be subjected to one or more processing steps aimed at achieving an adequate measurement of the marker.

In other example embodiments, detection of a marker may include immunological assay methods, wherein the ability of an assay to separate, detect and/or quantify a marker (such as, preferably, peptide, polypeptide, or protein) is conferred by specific binding between a separable, detectable and/or quantifiable immunological binding agent (antibody) and the marker. Immunological assay methods include without limitation immunohistochemistry, immunocytochemistry, flow cytometry, mass cytometry, fluorescence activated cell sorting (FACS), fluorescence microscopy, fluorescence based cell sorting using microfluidic systems, immunoaffinity adsorption based techniques such as affinity chromatography, magnetic particle separation, magnetic activated cell sorting or bead based cell sorting using microfluidic systems, enzyme-linked immunosorbent assay (ELISA) and ELISPOT based techniques, radioimmunoassay (RIA), Western blot, etc.

In certain example embodiments, detection of a marker or signature may include biochemical assay methods, including inter alia assays of enzymatic activity, membrane channel activity, substance-binding activity, gene regulatory activity, or cell signaling activity of a marker, e.g., peptide, polypeptide, protein, or nucleic acid.

In other example embodiments, detection of a marker may include mass spectrometry analysis methods. Generally, any mass spectrometric (MS) techniques that are capable of obtaining precise information on the mass of peptides, and preferably also on fragmentation and/or (partial) amino acid sequence of selected peptides (e.g., in tandem mass spectrometry, MS/MS; or in post source decay, TOF MS), may be useful herein for separation, detection and/or quantification of markers (such as, preferably, peptides, polypeptides, or proteins). Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000, ISBN 089603609x; Biemann 1990. Methods Enzymol 193: 455-79; or Methods in Enzymology, vol. 402: “Biological Mass Spectrometry”, by Burlingame, ed., Academic Press 2005, ISBN 9780121828073) and may be used herein. MS arrangements, instruments and systems suitable for biomarker peptide analysis may include, without limitation, matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; atmospheric pressure photoionization mass spectrometry (APPI-MS); APPI-MS/MS; and APPI-(MS)n. Peptide ion fragmentation in tandem MS (MS/MS) arrangements may be achieved using manners established in the art, such as, e.g., collision induced dissociation (CID). Detection and quantification of markers by mass spectrometry may involve multiple reaction monitoring (MRM), such as described among others by Kuhn et al. 2004 (Proteomics 4: 1175-86). MS peptide analysis methods may be advantageously combined with upstream peptide or protein separation or fractionation methods, such as for example with the chromatographic and other methods.

In other example embodiments, detection of a marker may include chromatography methods. In a one example embodiment, chromatography refers to a process in which a mixture of substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography may be columnar. While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993. Exemplary types of chromatography include, without limitation, high-performance liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immunoaffinity, immobilised metal affinity chromatography, and the like.

In certain embodiments, further techniques for separating, detecting and/or quantifying markers may be used in conjunction with any of the above described detection methods. Such methods include, without limitation, chemical extraction partitioning, isoelectric focusing (IEF) including capillary isoelectric focusing (CIEF), capillaryisotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

In certain examples, such methods may include separating, detecting and/or quantifying markers at the nucleic acid level, more particularly RNA level, e.g., at the level of hnRNA, pre-mRNA, mRNA, or cDNA. Standard quantitative RNA or cDNA measurement tools known in the art may be used. Non-limiting examples include hybridization-based analysis, microarray expression analysis, digital gene expression profiling (DGE), RNA-in-situ hybridization (RISH), Northern-blot analysis and the like; PCR, RT-PCR, RT-qPCR, end-point PCR, digital PCR or the like; supported oligonucleotide detection, pyrosequencing, polony cyclic sequencing by synthesis, simultaneous bi-directional sequencing, single-molecule sequencing, single molecule real time sequencing, true single molecule sequencing, hybridization-assisted nanopore sequencing, sequencing by synthesis, single-cell RNA sequencing (sc-RNA seq), or the like. By means of an example, methods to profile the RNA content of large numbers of individual cells have been recently developed. The cell of origin is determined by a cellular barcode. In certain embodiments, special microfluidic devices have been developed to encapsulate each cell in an individual drop, associate the RNA of each cell with a ‘cell barcode’ unique to that cell/drop, measure the expression level of each RNA with sequencing, and then use the cell barcodes to determine which cell each RNA molecule came from. In these regards, reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO 2014210353 A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; and Rosenberg et al., 2017, “Scaling single cell transcriptomics through split pool barcoding” bioRxiv preprint first posted online Feb. 2, 2017, doi: dx.doi.org/10.1101/105163, all the contents and disclosure of each of which are herein incorporated by reference in their entirety.

In certain embodiments, the invention involves single nucleus RNA sequencing. In this regard, reference is made to Swiech et al., 2014, “In vivo interrogation of gene function in the mammalian brain using CRISPR-Cas9” Nature Biotechnology Vol. 33, pp. 102-106; and Habib et al., 2016, “Div-Seq: Single-nucleus RNA-Seq reveals dynamics of rare adult newborn neurons” Science, Vol. 353, Issue 6302, pp. 925-928, both of which are herein incorporated by reference in their entirety.

The methods of diagnosing optionally comprise detected whether gene expression profile is overexpressed compared to a cell or tissue that is not infected, or whether the gene expression profile is underexpressed compared to a cell or tissue that is not infected. In some instances, the cell or tissue in the subject and the cell or tissue that is not infected is of the same cell type or tissue type. In some embodiments, the gene expression profile is correlated with the copy number of TB in the cell. In some instances, the gene expression profile can be indicative of higher copy number or lower copy number of TB within a cell. The step of detecting overexpression or under expression of particular genes can be performed simultaneously with a gene expression profile. Alternatively, presence of particular genes can be detected initially, with further comparison and/or quantitation, including computation of overexpression and under expression occurring subsequent to initial detection.

### Theragnostics

Upon diagnosis of an infection, optionally including determination of latent or active infection and/or relative copy numbers of mycobacteria in cells or tissue, treatment regimens can be administered.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

An effective amount or therapeutically effective amount can refer to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

Considerations for latent TB infection treatment can include the subject to be treated, including whether the subject has a suppressed or lowered immune systems, including HIV-infected persons, organ transplant recipients, young children, and other persons who are immunosuppressed (e.g. taking the equivalent of >15 mg/day of prednisone for 1 month or longer, taking TNF-a antagonists. Other persons to consider for latent infection treatment can include persons with fibrotic changes on chest radiograph consistent with old TB, recent contacts of an individual with TB, residents and employees of high-risk settings (e.g. nursing homes, homeless shelters, health care facilities), mycobacteriology laboratory personnel, persons from high-prevalence countries and injection drug users.

Several regimens for latent infections are recommended, and can depend on other factors, including health and age of the subject (cdc.gov Treatment Options for Latent Tuberculosis Infection, incorporated herein by reference). Treatment regimens can include isoniazid (INH) for a duration of 6 months or 9 months, requiring a minimum number of doses administered. INH can be administered 9 months daily (270 minimum doses) and is a standard treatment regimen, preferred for HIV-infected people taking antiretroviral therapy, and children aged 2-11; or INH can be administered twice weekly over 9 months (76 minimum doses). INH can also be used in certain instances for 6 months, administered daily (180 minimum doses) or twice weekly (52 minimum doses). INH can also be used in combination with Rifapentine for a duration of three months, dosed once weekly for a minimum of 12 doses. Rifampin (RIF) is recommended administered daily for 4 months for at least 120 minimum doses. In some instances, treatment for latent infection can be based on the methods of detection provided herein, including use of the gene expression signatures as detailed in Tables 1 and 2.

Treatment for active infections can, in some embodiments, include additional testing to determine if the TB infection is drug susceptible or drug resistant prior to treatment. When drug susceptible, a combination of drugs including ethambutol, INH, pyrazinamide and RIF can be used for an initial intensive phase of treatment, followed by administration of INH and RIF for a continuation phase usually given for either 4 or 7 months. Drug-resistant TB, multidrug-resistant TB and extensively drug-resistant TB may require combinations of first-line treatments as discussed, as well as floroquinolones, bedaquiline fumarate, ofloxacin, cycloserine, and/or including injectable second-line drugs such as amikacin, kanamycin, or capreomycin. In some instances, treatment for active infection can be based on the methods of detection provided herein, including use of the gene expression signatures as detailed in Tables 1 and 2.

In some embodiments, the treatment can include administering one or modulating agents of a host gene or gene products from the genes listed in Tables 1 and 2, or modulation of a gene or pathways as disclosed herein and as detailed in the examples. Treatment can be based in whole or in part on characterizations of copy number per cell or population of cell; the genes, gene products and pathways associated with multiplicity of infection that are detected, the degree of under expression or overexpression of certain genes or gene products, or the relative number of differentially expressed genes or gene products.

In some embodiments, methods of monitoring treatment of a M. tuberculosis infection in a subject is provided. Methods of monitoring may comprise one or more steps of detecting, in some instance, at time intervals. The time intervals may be prior to infection and subsequent to infection, during an active infection, prior to treatment and subsequent to beginning treatment, or some combination thereof.

### Monitoring Disease

the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

The invention provides a method for monitoring infection in a subject and for determining the severity of a disease or condition by comparing the gene profiles from a healthy subject or reference control with one from a subject suspected of having a disease or condition, or monitoring the progression of the disease.

Method embodiments are also provided for monitoring a subject having no symptoms of disease to determine onset of or diagnose a disease comprising implanting the detector unit on or in the subject and monitoring changes, or velocity of change in the level or presence of one or more biomolecule markers associated with the disease wherein a change, or alterations in velocity of change in the level or presence of the one or more biomolecule markers indicates presence of the disease.

In another aspect, a method is provided for monitoring a subject to predict response to treatment for a disease comprising implanting the detector unit on or in the subject and monitoring changes in the level or presence of one or more biomolecule markers associated with a disease wherein a change, or alterations in velocity of change in the level or presence of the one or more biomolecule markers associated with treatment resistance of the disease indicates the presence or absence of resistance of the subject to a disease treatment.

The step of detecting for the purposes of monitoring can, in one embodiment, comprise whether one or more genes is overexpressed compared to a cell that is not infected. The step of detecting can, in one embodiment, comprise whether one or more genes is underexpressed compared to a cell that is not infected. The step of detecting can also comprise a gene expression profile of one or more genes, as described herein, and may include overexpressed and underexpressed genes in the gene expression profile.

In one embodiment, the change in the level or presence of the one or more biomolecule markers associated with the disease is compared to normal levels in the subject or a population of healthy or normal subjects where the change, or alterations in velocity of change in the level or presence of the one or more biomolecules indicates the presence of the disease.

### Examples of Diseases

The methods and compositions herein may be used for treating, preventing, and diagnosing a variety of diseases.

### Infectious Diseases

In some embodiments, the diseases may be infectious diseases. In one example, the disease is tuberculosis, e.g., caused by Mycobacterium tuberculosis.

Infections or infectious diseases include viral infectious diseases, such as AIDS, Chickenpox (Varicella), Common cold, Cytomegalovirus Infection, Colorado tick fever, Dengue fever, Ebola hemorrhagic fever, Hand, foot and mouth disease, Hepatitis, Herpes simplex, Herpes zoster, HPV, Influenza (Flu), Lassa fever, Measles, Marburg hemorrhagic fever, Infectious mononucleosis, Mumps, Norovirus, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease and Yellow fever; bacterial infectious diseases, such as Anthrax, Bacterial Meningitis, Botulism, Brucellosis, Campylobacteriosis, Cat Scratch Disease, Cholera, Diphtheria, Epidemic Typhus, Gonorrhea, Impetigo, Legionellosis, Leprosy (Hansen's Disease), Leptospirosis, Listeriosis, Lyme disease, Melioidosis, Rheumatic Fever, MRSA infection, Nocardiosis, Pertussis (Whooping Cough), Plague, Pneumococcal pneumonia, Psittacosis, Q fever, Rocky Mountain Spotted Fever (RMSF), Salmonellosis, Scarlet Fever, Shigellosis, Syphilis, Tetanus, Trachoma, Tuberculosis, Tularemia, Typhoid Fever, Typhus and Urinary Tract Infections; parasitic infectious diseases, such as African trypanosomiasis, Amebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amebic infection, Giardiasis, Gnathostomiasis, Hymenolepiasis, Isosporiasis, Kala-azar, Leishmaniasis, Malaria, Metagonimiasis, Myiasis, Onchocerciasis, Pediculosis, Pinworm Infection, Scabies, Schistosomiasis, Taeniasis, Toxocariasis, Toxoplasmosis, Trichinellosis, Trichinosis, Trichuriasis, Trichomoniasis and Trypanosomiasis; fungal infectious disease, such as Aspergillosis, Blastomycosis, Candidiasis, Coccidioidomycosis, Cryptococcosis, Histoplasmosis, Tinea pedis (Athlete's Foot) and Tinea cruris; prion infectious diseases, such as Alpers' disease, Fatal Familial Insomnia, Gerstmann-Straussler-Scheinker syndrome, Kuru and Variant Creutzfeldt-Jakob disease.

The diseases may be caused by a microbial infection, e.g., by bacteria, viruses, fungi, and other microorganisms. Examples of infectious bacteria (including mycobacteria) include Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, Actinomyces israelli, and Salmonella spp. Examples of infectious fungi include: Cryptococcus neoformans, Histoplasma capsulatum, Coccidioides immitis, Blastomyces dermatitidis, Chlamydia trachomatis, and Candida albicans.

### Immune-Related Diseases

The diseases may be immune-related diseases. Immune-related diseases include those diseases, diseases or conditions that have an immune component and those that are substantially or entirely immune system-mediated. Autoimmune diseases are those wherein the animal's own immune system mistakenly attacks itself, thereby targeting the cells, tissues, and/or organs of the animal's own body. For example, the autoimmune reaction is directed against the nervous system in multiple sclerosis and the gut in Crohn's disease. In other autoimmune diseases such as systemic lupus erythematosus (lupus), affected tissues and organs may vary among individuals with the same disease. One person with lupus may have affected skin and joints whereas another may have affected skin, kidney, and lungs. Ultimately, damage to certain tissues by the immune system may be permanent, as with destruction of insulin-producing cells of the pancreas in Type 1 diabetes mellitus. Specific autoimmune diseases that may be ameliorated using the compounds and methods of this invention include without limitation, autoimmune diseases of the nervous system (e.g., multiple sclerosis, myasthenia gravis, autoimmune neuropathies such as Guillain-Barre, and autoimmune uveitis), autoimmune diseases of the blood (e.g., autoimmune hemolytic anemia, pernicious anemia, and autoimmune thrombocytopenia), autoimmune diseases of the blood vessels (e.g., temporal arteritis, anti-phospholipid syndrome, vasculitides such as Wegener's granulomatosis, and Behcet's disease), autoimmune diseases of the skin (e.g., psoriasis, dermatitis herpetiformis, pemphigus vulgaris, and vitiligo), autoimmune diseases of the gastrointestinal system (e.g., Crohn's disease, ulcerative colitis, primary biliary cirrhosis, and autoimmune hepatitis), autoimmune diseases of the endocrine glands (e.g., Type 1 or immune-mediated diabetes mellitus, Grave's disease. Hashimoto's thyroiditis, autoimmune oophoritis and orchitis, and autoimmune disease of the adrenal gland); and autoimmune diseases of multiple organs (including connective tissue and musculoskeletal system diseases) (e.g., rheumatoid arthritis, systemic lupus erythematosus, scleroderma, polymyositis, dermatomyositis, spondyloarthropathies such as ankylosing spondylitis, and Sjogren's syndrome). In addition, other immune system mediated diseases, such as graft-versus-host disease and allergic diseases, are also included in the definition of immune diseases herein. Because a number of immune diseases are caused by inflammation, there is some overlap between diseases that are considered immune diseases and inflammatory diseases. For the purpose of this invention, in the case of such an overlapping disease, it may be considered either an immune disease or an inflammatory disease.

Immune related diseases may include allergic diseases. The term “allergic disease” means a disease, condition or disease associated with an allergic response against normally innocuous substances. These substances may be found in the environment (such as indoor air pollutants and aeroallergens) or they may be non-environmental (such as those causing dermatological or food allergies). Allergens can enter the body through a number of routes, including by inhalation, ingestion, contact with the skin or injection (including by insect sting). Many allergic diseases are linked to atopy, a predisposition to generate the allergic antibody IgE. Because IgE is able to sensitize mast cells anywhere in the body, atopic individuals often express disease in more than one organ. For the purpose of this invention, allergic diseases include any hypersensitivity that occurs upon re-exposure to the sensitizing allergen, which in turn causes the release of inflammatory mediators. Allergic diseases include without limitation, allergic rhinitis (e.g., hay fever), sinusitis, rhinosinusitis, chronic or recurrent otitis media, drug reactions, insect sting reactions, latex reactions, conjunctivitis, urticaria, anaphylaxis and anaphylactoid reactions, atopic dermatitis, asthma and food allergies.

Immune related diseases may include inflammatory diseases. An “inflammatory disease” means a disease, disease or condition characterized by inflammation of body tissue or having an inflammatory component. These include local inflammatory responses and systemic inflammation. Examples of such inflammatory diseases include: transplant rejection, including skin graft rejection; chronic inflammatory diseases of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung diseases such as asthma, adult respiratory distress syndrome, and chronic obstructive airway disease; inflammatory diseases of the eye including corneal dystrophy, trachoma, onchocerciasis, uveitis, sympathetic ophthalmitis and endophthalmitis; chronic inflammatory diseases of the gums, including gingivitis and periodontitis; tuberculosis; leprosy; inflammatory diseases of the kidney including uremic complications, glomerulonephritis and nephrosis; inflammatory diseases of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis; autoimmune diseases, immune-complex vasculitis, systemic lupus and erythematodes; systemic lupus erythematosus (SLE); and inflammatory diseases of the heart such as cardiomyopathy, ischemic heart disease hypercholesterolemia, atherosclerosis; as well as various other diseases with significant inflammatory components, including preeclampsia; chronic liver failure, brain and spinal cord trauma, and cancer. There may also be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent used in cancer chemotherapy.

Immune related diseases may include autoimmune diseases. Examples of autoimmune conditions include rheumatoid arthritis, rheumatoid spondylitis, osteoarthritis and gouty arthritis, allergies, multiple sclerosis, autoimmune diabetes, autoimmune uveitis, Kidney syndrome, multi-system autoimmune diseases, autoimmune hearing loss, type I diabetes, ankylosing spondylitis, Behcet's syndrome, dermatomyositis, Graves' disease, juvenile rheumatoid arthritis, multiple sclerosis, psoriatic arthritis, Reiter's syndrome, rheumatoid arthritis, Sjogren's syndrome, systemic lupus erythematosus, Wegener's granulatomatosis, myasthenia gravis, ankylosing spondylitis, celiac disease, Crohn's disease, Hashimoto's thyroiditis, or autoimmune uveitis), graft versus host disease, and allograft rejection.

### Cancers

The diseases may be cancers, e.g., solid tumors. The cancer may include solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

Cancers in which expression of an EMT program occurs may include, breast cancer, colon cancer, lung cancer, prostate cancer, testicular cancer, brain cancer, skin cancer, rectal cancer, gastric cancer, esophageal cancer, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers. Thus, the disclosure is generally applicable to any type of cancer in which expression of an EMT program occurs.

The cancer may include liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

### Injury and Wound Healing

The methods and compositions may be used for treating injuries and wounds, and modulate the wound healing process. The injury herein may be an acute injury. As used herein, the term “acute injury” includes injuries that have occurred suddenly or recently occurred. For example, an acute injury may have occurred suddenly, e.g., due to a traumatic event (external or internal), infections (e.g., caused by bacterial viruses, fungi and parasites), stroke (cerebral circulatory disturbance and intracerebral or subarachnoid haemorrhage), intoxications, and traumatic lesions. The injury herein may be a chronic injury or disease. As used herein, the term “chronic injury” an injury disease that has a slow, insidious onset and generally a long duration.

The present application also provides aspects and embodiments as set forth in the following numbered Statements:

1. A method for treatment or prophylaxis of a Mycobacterium tuberculosis (MTB) infection comprising modulating expression of one or more genes in a mast cell, a plasmablast, or a combination thereof, in a subject in need thereof, wherein the one or more genes expresses at a level different in a resolving granuloma in an MTB infected tissue comparing to a level in a progressing granuloma in the MTB infected tissue.

2. The method of statement 1, wherein the modulating comprises upregulating the expression of the one or more genes, wherein the one or more genes expresses at a higher level in a resolving granuloma in an MTB infected tissue comparing to a level in a progressing granuloma in the MTB infected tissue.

3. The method of statement of 1 or 2, wherein the modulating comprises downregulating the expression of the one or more genes, wherein the one or more genes expresses at a lower level in a resolving granuloma in an MTB infected tissue comparing to a level in a progressing granuloma in the MTB infected tissue.

4. The method of any one of the statements above, wherein the modulating comprises delivering one or more agonists of the one or more genes to a subject.

5. An engineered mast cell or plasmablast comprising elevated expression of one or more genes that expresses at a level higher in a resolving granuloma in an MTB infected tissue comparing to a level in a progressing granuloma in the MTB infected tissue.

6. An engineered mast cell or plasmablast comprising reduced expression of one or more genes that expresses at a level lower in a resolving granuloma in an MTB infected tissue comparing to a level in a progressing granuloma in the MTB infected tissue.

7. A method of identifying a population of cells correlating to a granuloma characteristic, the method comprising: a. obtaining a first plurality of cells from one or more granuloma with the characteristic and a second plurality of cells from one or more granuloma without the characteristic; b. sequencing nucleic acid molecules in the first and the second pluralities of cells using single cell sequencing; c. clustering genes differently expressed between the first and the second plurality of cells; and d. identifying the population of cells based on the clustering of the different expressed genes.

8. The method of statement 7, further comprising excluding a cluster of genes expressing only in a single granuloma.

9. The method of statement 7 or 8, further comprising identifying the population of cells in different subjects.

10. The method of any one of statements 7-9, wherein the granuloma characteristic is progressiveness.

11. A method of determining a characteristic of a granuloma in a subject infected by MTB, the method comprising identifying the population of cells according to any one of statements 7-10.

12. A method of treatment or prophylaxis of a Mycobacterium tuberculosis (MTB) infection comprising activating a p53 pathway in macrophages of or from a patient in need thereof.

13. A method of treatment or prophylaxis of a Mycobacterium tuberculosis (MTB) infection comprising delivering a p53 agonist to a patient in need thereof.

14. A method of treatment or prophylaxis of a Mycobacterium tuberculosis (MTB) infection comprising delivering a p53 agonist to a patient's macrophages.

15. The method of any one of statements 12-14, wherein the p53 agonist is a p53 pathway agonist.

16. The method of statement 15, wherein the p53 agonist is delivered to the patient's macrophages in vivo.

17. The method of any one of statements 12-16, wherein the macrophages are activated or agonized in vivo.

18. The method of any one of statements 12-17, wherein the macrophages are activated or agonized in a sample from the patient or ex vivo, and optionally, subsequently returned to the patient.

19. The method of any one of statements 12-18, wherein the p53 agonist is delivered to the patient's macrophages in a sample from the patient or ex vivo, and optionally, subsequently returned to the patient.

20. The method of any one of statements 12-19, wherein the p53 pathway activator, p53 agonist, or p53 pathway agonist is an MDM2 inhibitor.

21. The method of any one of statements 12-20, wherein the MDM2 inhibitor is nutilin-3a.

22. The method of any one of statements 12-21, wherein control of MTB infection by macrophages in the patient is promoted.

23. The method of any one of statements 12-22, wherein the macrophage is, or is derived from, a primary human CD14+ monocyte-derived macrophage (MDM).

24. The method of any one of statements 12-23, wherein at least one of the following genes are upregulated: TOP2B, SORT1, NUDT3, IRF4, CXCL1.

25. A method of differentiating one or more macrophage subpopulations infected by MTB from one or more uninfected macrophage subpopulations, the method comprising: a. assaying the macrophages for the presence, or overexpression compared to wt macrophages, of: i. at least one of cytokine receptors (including IFNGR1, IL1RN), SLAM family members (including SLAM7, SLAMF5), and kinases (including HCK, CAMK1), or ii. at least one of differentiators of macrophage state, including M1 and M2, HLA-DRB1, and CD68, in particular CD86; b. assaying the macrophages for the presence, or overexpression compared to wt macrophages, of at least one of: i. at least one of ApoE, CD36, CD52, and IL-8 ApoE, CD36, CD52, IL8, c. identifying the one or more infected macrophage subpopulations based on the assay in a); d. identifying the one or more uninfected macrophage subpopulations based on the assay in b); and optionally, e. separating the one or more infected macrophage subpopulations from the one or more uninfected macrophage subpopulations based on the identifications made in c) and d); wherein separating optionally comprises i. by labelling or tagging one of the infected or the uninfected subpopulations; or ii. by differentially labelling or tagging the infected and the uninfected subpopulations.

26. The method of statement 25, wherein identified, and optionally separated, infected macrophage subpopulations are contacted with a p53 agonist or p53 pathway agonist to promote a control phenotype.

27. The method of treatment of a Mycobacterium tuberculosis (MTB) infection of any one of statements 12-26, comprising activating the p53 pathway in macrophages of or from the patient to promote a control phenotype.

28. A method prophylaxis of a Mycobacterium tuberculosis (MTB) infection, comprising activating a p53 pathway in macrophages of or from a patient exposed to or at risk of MTB infection, optionally to promote a control phenotype.

29. A method of treatment or prophylaxis of an Mycobacterium tuberculosis (MTB) infection comprising activating a NF-κB pathway in macrophages of or from a patient in need thereof.

30. A method of treatment or prophylaxis of a Mycobacterium tuberculosis (MTB) infection comprising activating a Vitamin D Receptor (VDR) pathway in macrophages of or from a patient in need thereof.

31. A CD14+ macrophage model cell or cell line, wherein: at least one of the following genes are upregulated: CD206, CD86, CD32; and/or at least one of the following genes are downregulated: CD163.

32. The model cell or cell line of any one of statements 12-31, which is or is derived from a primary human CD14+ monocyte-derived macrophage (MDM).

33. A method of treating or preventing a disease by modulating a microenvironment of a cell or cell mass in a subject, the method comprising administrating an effective amount of one or more modulating agents that modulate mast cells, plasma cells, Th1-Th17 cells, and/or CD8+ T cells in the subject.

34. The method of statement 33, wherein the modulating agent reduce number or function of mast cells.

35. The method of any one of statements 33-34, wherein the one or more modulating agents modulates expression of one or more genes in mast cells.

36. The method of any one of statements 33-35, wherein the one or more genes in mast cells comprises genes in IL-13 signaling pathway and/or genes in IL-33 signaling pathway.

37. The method of any one of statements 33-36, wherein the one or more genes in mast cells comprises IL-33, IL-1R1, and/or IL-13.

38. The method of any one of statements 33-37, wherein the one or more modulating agents increase number or function of Th1-Th17 cells.

39. The method of any one of statements 33-38, wherein the one or more genes in Th1-Th17 cells.

40. The method of any one of statements 33-39, wherein the one or more genes in Th1-Th17 cells comprises genes in INF-γ signaling pathway and/or genes in TGFβ signaling pathway.

41. The method of any one of statements 33-40, wherein the one or more genes in Th1-Th17 cells comprises INF-γ, INF-γ receptor 2, TGFβ1, TGFβ receptor 3, CCL5, and/or IL-23R.

42. The method of any one of statements 33-41, wherein the one or more genes in Th1-Th17 cells comprises IL-2RG, IFN-γ, IFI27, LAG3, TIGIT, CD8A, NKG7, CCL20, CCL3, and/or CCL5.

43. The method of any one of statements 33-42, wherein the one or more modulating agents modulates expression of: a. GZMA, GZMB, GNLY, and PRF1, b. CCR7, LEF1, and SELL in Naïve T cells, c. FOXP3, IKZF2, and IL1RL1 in regulatory T cells, d. OAS2, MX1, and ISG15 in interferon-responsive cells, e. GZMK, CCL5, and CXCR4 in CD8+ T cells, f. CX3CR1, GZMB, and ZEB2 in CD8+ T cells, g. MKI67 and TOP2A in proliferating T cells, h. APOC1, APOE, and C1QB in alveolar macrophages, i. TIMP1 and IDO1 in monocytes, j. LIPA and MAN2B1 in macrophages, k. MRC1, FABP5, and PPARG in lipid-laden macrophages, l. CP, CXCL9, and NFKB1 in inflammatory macrophages, m. MKI67 and TOP2A in proliferating macrophages, n. BIRC3, CCR7, and LAMP3 in myeloid dendritic cells, o. BHLHE40, SATB1 and RBPJ in Th17 cells, p. IFNG, CCL4, RORC, IL17A, IL17F, IL1R1, RORA, IRF4, and RBPJ in Th17 cells, q. IL23R, IL7R, NDFIP1, ILI1R1, RORA, IRF4, and RBPJ in Ex-Th17 cells, r. KLF2, TGFBR3, CX3CR1, and GZMB in CD8+ T cells, s. FOXP3, TIGIT, GITR, and GATA3 in ST2+ regulatory T cells, t. IL2RG, IFNG, IFI27, LAG3, TIGIT, CD8A, NKG7, CCL20, CCL3, and CCL5 in Th1-Th17 cell, or u. any combination thereof.

44. The method of any one of statements 33-43, wherein the one or more modulating agents is comprised in a vaccine formulation.

45. The method of any one of statements 33-44, wherein the disease is bacterial infection, tuberculosis, cancer, chronic rhinosinusitis, asthma, allergy, wound, or a combination thereof.

46. The method of any one of statements 33-45, wherein the disease is a latent disease.

47. The method of any one of statements 33-46, wherein the disease is an active disease.

48. The method of any one of statements 33-47, wherein the cell or cell mass is a granuloma.

49. The method of any one of statements 33-48, wherein the one or more modulating agents comprises an antibody, or antigen binding fragment, an aptamer, affimer, non-immunoglobulin scaffold, small molecule, genetic modifying agent, or a combination thereof.

50. A method of treating a disease in a subject comprising: a. contacting one or more mast cells and/or Th1-Th17 cells with one or more modulating agents, wherein the one or more modulating agents activates i. IL-33, IL-1R1, and genes in IL-13 signaling pathway in the mast cells, and/or ii. INF-γ, INF-γ receptor 2, TGFβ1, TGFβ receptor 3, CCL5, and genes in INF-γ and TGFβ signaling pathway in the Th1-Th17 cells; b. administering the mast cells and/or Th1-Th17 from a) to the subject.

51. The method of statement 50, wherein the mast cells and/or Th1-Th17 cells are isolated or derived from the subject.

52. A mast cell or cell line expressing one or more of: IL-33, IL-1R1, and genes in IL-13 signaling pathway.

53. A Th1-Th17 cell expressing one or more of: INF-γ, INF-γ receptor 2, TGFβ1, TGFβ receptor 3, CCL5, and genes in INF-γ and TGFβ signaling pathway.

54. A vaccine comprising the one or more modulating agents in any one of statements 33 to 51.

55. A pharmaceutical formulation or vaccine comprising one or more modulating agents or cell or cell line in any one of statements 1 to 54 for use as a medicament.

56. A pharmaceutical formulation or vaccine comprising one or more modulating agents or cell or cell line in any one of statements 1 to 54 for use in the treatment of a disease.

The invention is further described in the following examples, which do not limit the scope of the invention described in the claims.

### EXAMPLES

### Example 1

Several human pathogens—including Mycobacterium tuberculosis (Mtb)—survive in macrophages, innate immune cells that represent the first line of defense against invading intracellular organisms. In this example, Applicants leveraged high-throughput single-cell RNA sequencing to define the emergent heterogeneity in the transcriptional responses of human macrophages to Mtb infection. Applicants found that infection activated different transcriptional networks in distinct subpopulations of cells, and that the activity of these networks correlated with control of Mtb. Using this data, Applicants nominated and tested small molecules that manipulate these transcriptional networks to enhance bacterial control.

Macrophages may control Mtb infection but have only modest capacity to kill the bacterium1. Mechanistically, Mtb survival inside macrophages has been attributed to its ability to disrupt phagolysosomal maturation as well scavenge nutrients from the host. However, it has recently been appreciated that the limited bacterial control exerted by a population of human macrophages may reflect the aggregate effects of simultaneous bacterial clearance and growth. How and where this variation emerges during the interaction between bacterium and pathogen, and whether there are features that distinguish macrophages that successfully clear Mtb from those that do not, remained poorly understood.

Differences between macrophages have been understood in terms of polarization state; however, several recent studies point to a much more complex model integrating differentiation, activation, and tissue cues as signals that can influence macrophage state2. However, these paradigms, which have largely been understood using bulk approaches, do not explain the emergent heterogeneity in macrophage functional state that is apparent during Mtb infection. Recently, single-cell transcriptional profiling and dynamic imaging of myeloid cells has revealed surprising cell-to-cell variability in basal transcription and the responses evoked by purified toll-like receptor (TLR) stimuli despite homogeneous culture conditions3-5. It is unknown whether these variations are propagated into durable differences in macrophage fate or functional capacity.

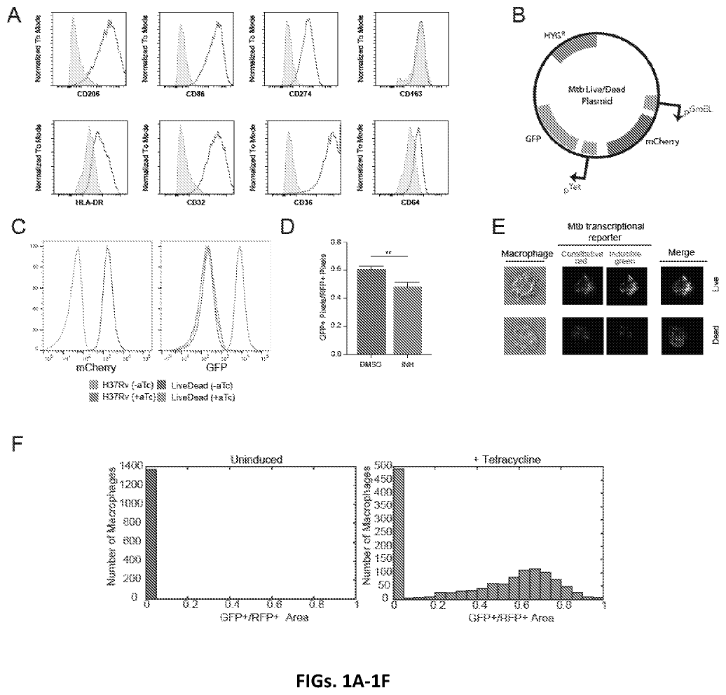

To understand the intrinsic capacity of macrophages to clear the bacterium, Applicants developed an in vitro model of Mtb infection. Primary human CD14+ monocyte-derived macrophages (MDMs) were matured for eight days in the absence of polarizing stimuli (Methods). By flow cytometry, these cells were characterized by high expression of CD206, CD86, and CD32, and low expression of CD163 (FIG. 1A). To track bacterial fate, Applicants used Mtb expressing a live-dead reporter (FIGS. 1B, 1C), allowing assessment of bacterial survival at the single-cell level by high-throughput quantitative microscopy. To test the performance of the reporter strain, Applicants infected MDMs at a low multiplicity of infection (MOI) with the Mtb reporter strain and treated with the microbicidal antibiotic isoniazid, which resulted in a decrease in the GFP+/RFP+ pixel area that correlated well with bacterial killing as measured by CFU albeit with a compressed dynamic range (FIG. 1D).

Applicants next focused on macrophage control of Mtb in the first five days post-infection as there was little evidence of macrophage death as measured by LDH release in this time period. Despite homogenous induction of the transcriptional reporter in broth, single-cell analysis of bacterial fate revealed a population of macrophages that harbored transcriptionally silent bacteria (RFPhi, GFPlo) and a population of macrophages that harbored transcriptionally active bacteria (RFPhi, GFPhi), suggesting the emergence of restrictive and permissive macrophage subpopulations (FIGS. 1D, 1D).

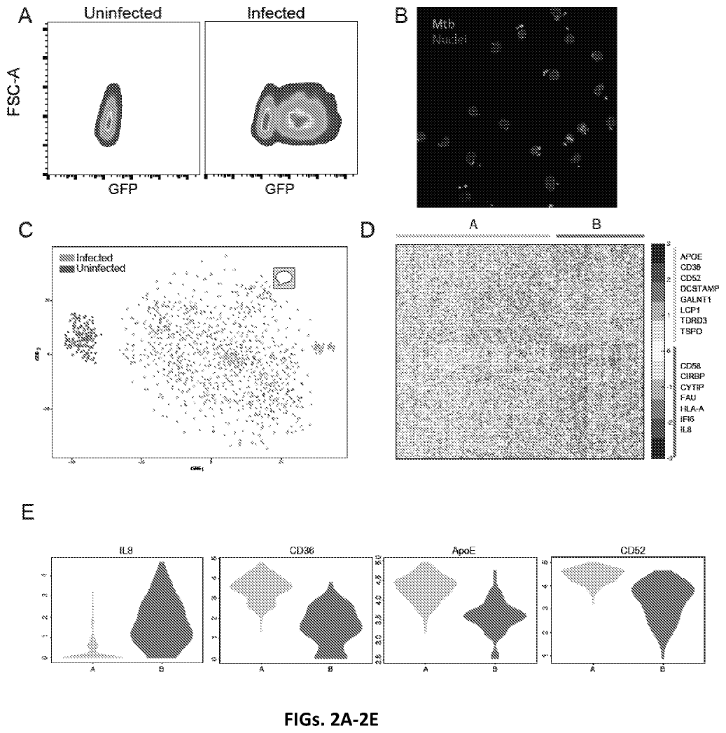

To test whether heterogeneity in the initial transcriptional responses of macrophages to infection gives rise to differences in antimicrobial capacity, Applicants used single-cell RNA sequencing to characterize the transcriptional responses of Mtb-infected macrophages at an early time point (24 hr) following infection. To sample the number of cells required to robustly define the subpopulation architecture of those response, Applicants employed Seq-Well, a single-cell transcriptional profiling platform that allows RNA-seq analysis of thousands of infected cells in challenging settings such as a BSL3 facility6. Macrophages were infected at very low multiplicity of infection (MOI) with Mtb expressing GFP, such that most cells contained zero or one bacterium. Applicants isolated infected cells by fluorescence activated cell sorting and confirmed that sorted cells were homogeneously infected at a low MOI by microscopy (FIGS. 2A, 2B). At 24 hours post-infection, approximately 12,000 GFP positive cells were loaded onto a Seq-Well array for single-cell RNA sequencing analysis. After exclusion of low-quality cells (<5,000 unique transcripts), we analyzed a total of 2053 infected cells. To assess pre-existing heterogeneity in the macrophage population, 443 cells from the same donor that were not subject to infection but otherwise processed similarly were analyzed in parallel (uninfected).

Clustering of uninfected and infected macrophages using t-distributed stochastic neighbor embedding (t-SNE) clearly distinguished uninfected and infected cells (FIG. 2C). Iterative clustering of uninfected macrophages revealed two subpopulations of uninfected cells. Genes distinguishing uninfected clusters of cells included ApoE, CD36, CD52, and IL8 (FIG. 2D, 2E). These genes may modulate the innate immune response to bacterial ligands but do not represent canonical macrophage population markers nor do they directly map to any of the emergent clusters after infection7-9. Applicants focused on transcriptional heterogeneity in Mtb-infected cells, recognizing that pre-existing heterogeneity might contribute to the complexity of the immune response to Mtb infection.

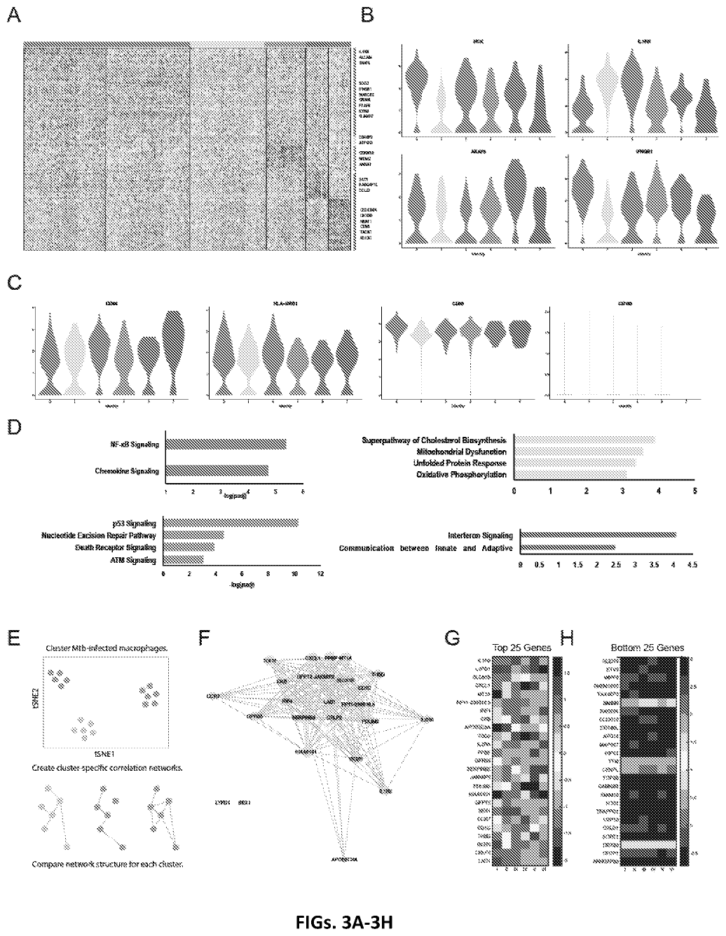

Despite transcriptional heterogeneity in the macrophages' resting state, it is possible that infection with a pathogen like Mtb is a sufficiently strong stimulus to drive a uniform response. However, in the infected macrophages, unsupervised clustering analysis revealed six (I-VI) distinct subpopulations of cells (FIG. 3A). Clustering the infected cells according to the cluster-defining genes from the uninfected cells did not confer subpopulation architecture to the infected population or vice versa. This suggests that there is an infection-mediated transition that diversifies population architecture. The genes that differentiated the infected macrophage subpopulations include cytokine receptors (IFNGR1, IL1RN), SLAM family members (SLAM7, SLAMF5), and kinases (HCK, CAMK1) (FIGS. 3A and 3B). Of the genes commonly used to differentiate macrophage states (e.g. M1 and M2) including CD86, HLA-DRB1, and CD68, only CD86 conferred discriminatory power to distinguish one infected macrophage subpopulation (FIG. 3C).

Applicants next identified shared features amongst the cluster-defining genes using gene ontology analysis to identify candidate functional states that differ between macrophage subpopulations. Pathway analysis of the cluster defining gene sets identified functionally distinct programs including ones associated with metabolism and interferon signaling (FIG. 3D). One cluster was defined by putative NF-κB activation and chemokine signaling, suggestive of the canonical response to TLR stimulation10. Other clusters reflected interferon signaling and metabolic remodeling. These are both responses to infection that have been described in bulk assays in the setting of Mtb infection11-13. However, the data suggest that these responses are distinct paths that a given cell may pursue rather than simultaneous responses. Finally, one cluster was defined by an apparent DNA damage response and p53 activation.

To define the biological circuits that differentiate Mtb infected macrophage activity and drive population architecture, we subdivided our single-cell data based on cluster identity and constructed gene-gene correlation networks for each subpopulation of cells (Methods). Edges were drawn between genes with statistically significant correlation values resulting in six transcriptional networks (FIGS. 3E, 3F). To compare network architecture between clusters, Applicants calculated the eigenvector centrality of each gene in each of the networks as a way of estimating its relative importance in the correlation network. Applicants then compared the z-scored eigenvector centrality for each gene across clusters to assess conservation of network architecture across clusters (FIGS. 3G, 3H). Genes with conserved centrality across each cluster included TOP2B, SORT1, and NUDT3. Genes whose centrality varied considerably across clusters included IRF4 and CXCL1, both genes whose function is to tune the inflammatory environment and state of macrophages. Pathway analysis of the genes whose centrality was most variable across clusters indicated that these genes were enriched for NF-κB targets, suggesting that the variability in network structure was driven by the activity of this transcription factor. NF-κB mediated transcriptional responses are the dominant transcriptional network identified in Mtb-infected cells through bulk transcriptional profiling, but high expression of NF-kB target genes was found in a subpopulation of cells. Indeed, the other clusters of infected macrophages were defined in part by muted expression of NF-κB target genes. Previous studies using purified TLR stimuli have demonstrated that upon ligand stimulation, a small fraction of cells engage inflammatory responses at low doses or early time points, demonstrating TLR response inhomogeneity3,14. Furthermore, upon TLR stimulation, published studies describe nuclear oscillation of NF-kB with individual cells characterized by varying amplitude and total number of oscillations, which is correlated with distinct transcriptional programs4,15,16. These data provide a potential mechanism for variability in the expression of NF-kB regulated genes in the setting of Mtb infection.

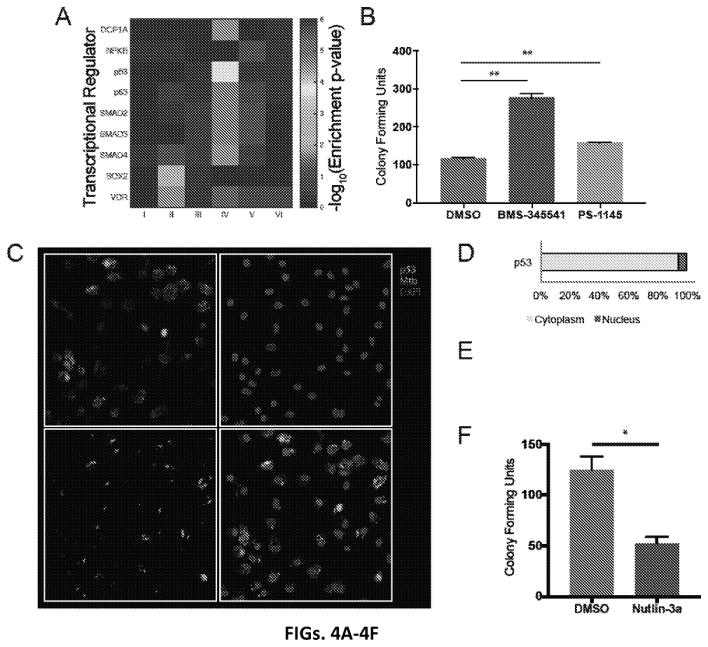

Applicants sought to infer additional transcription factors driving the cluster architecture of the Mtb infected macrophages through a gene set enrichment analysis (GSEA) of published chromatin binding data sets. Cluster II defining genes were highly enriched for NF-κB target genes, consistent with the gene-gene correlation network analysis (FIG. 4A). Cluster II defining genes were also enriched for genes regulated by the vitamin D receptor (VDR), which is broadly consistent with the known interactions between TLR2 signaling and vitamin D signaling that has been defined through bulk analyses17. Activation of NF-κB signaling may drive bacterial control via pro-inflammatory signaling. Consistent with this model, mice deficient in p50, an NF-KB subunit, quickly succumb to Mtb18. In the in vitro model, inhibiting the NF-κB transcriptional pathway using the I kappa B kinase inhibitors, BMS-345541 and PS-1145, worsened bacterial control (FIG. 4B).

In cluster IV, GSEA suggested enhanced activity of an alternative transcriptional circuit defined by high expression of p53 target genes. While p53 has been extensively studied and characterized in the setting of cancer and the DNA damage response, emerging data demonstrate a role for p53 in shaping immune responses19-21. Previous work in bulk assays has suggested that mycobacterial infection can modestly regulate p53 expression both at the transcript and protein level22. Applicants found evidence of subtle transcriptional regulation of p53 following Mtb infection in bulk analysis of infected macrophages, consistent with more robust regulation in a subpopulation of cells. As a complementary strategy, Applicants stained for p53 protein expression in Mtb infected macrophages. Consistent with the single-cell RNA sequencing analysis, we identified a subpopulation of infected macrophages with enrichment of p53 in the nucleus (FIGS. 4C, 4D).

Network analysis captures evidence for differential transcription factor activity. To assess how p53 contributes to bacterial control, Applicants treated infected cells with nutlin-3a, which chemically disrupts the interaction between p53 and MDM2, the ubiquitin ligase responsible for degradation of p5323,24. This resulted in enhanced expression of p53 (FIG. 4E). Strikingly, nutlin-3a treatment of Mtb infected macrophages significantly increased Mtb control without increasing macrophage death (FIGS. 4F, 4G). Future work will be required to dissect the antimicrobial mechanism by which this occurs.

Collectively, the data provided a molecular framework to interpret previous studies, which showed that macrophages exhibited variability in Mtb control and phagolysosomal fusion. The results suggested that not all macrophages were poised for execution of antimicrobial programs upon infection, despite an apparently homogeneous environment. Some macrophages activated defined NF-κB and VDR dependent responses. However, activation of distinct transcriptional modules in different subpopulations of infected cells co-occurred with muted NF-κB response, and indeed the majority of macrophages did not demonstrate sustained transcription of NF-κB-regulated genes.

The emergence of a dominant p53 response in a subpopulation of NF-κB low cells may reflect the extensive transcriptional crosstalk that has been described between the NF-κB and p53 pathways in cancer cells, where the relative level of each transcription factor may modulate the activity of the other. While NF-κB and p53 have a predominantly antagonistic relationship in tumor cells, their relationship appears more complex in immune circuits. Recent studies demonstrate that in primates, p53 is integrated into the TLR signaling network through promoter binding upstream of many TLR genes20. Like NF-κB, p53 also demonstrates a pattern of oscillatory nuclear localizations, suggesting an interesting hypothesis that the period, phase, and amplitude of NF-κB and p53 responses may influence whether they act collaboratively or antagonistically25.

These data highlight the discrete nature of the macrophage transcriptional response to Mtb infection. By transcription factor enrichment, Applicants identified a subpopulation of cells with high expression of p53-regulated genes, and observe variability in nuclear localization of p53 in infected macrophages. By analogy, genes associated with interferon signaling and metabolic remodeling were enriched for in specific cluster-defining gene sets. Interferon signaling and metabolic remodeling are both described as key macrophage responses to infection12,13. The data suggested specific subpopulations of macrophages may be producing or responding to type I interferons, or undergoing metabolic remodeling, as opposed to a uniform response across all Mtb-infected macrophages. These data reframed pathways identified in bulk analyses as parallel options for the macrophage, not a suite of responses that are simultaneously triggered in a single cell.

In other myeloid cells, there is also emergent heterogeneity in response to viral stimuli, suggesting potential conservation of this feature across all myeloid cells. Moreover, a pathogen like Mtb may engage in coordinated responses that reinforce or manipulate emergent macrophage states. Cell-to-cell heterogeneity in bacterial metabolic or replicative state could drive differences in macrophage responses. Finally, Applicants demonstrate through use of chemical tools, that biasing the activity of these transcription factors or their expression level can modulate Mtb survival. These studies laid a path towards rational design of combinatorial immunomodulatory strategies to improve macrophage clearance of pathogens.

Materials and Methods

Mycobacterium tuberculosis (Mtb) culture—3583:eGFP H37Rv or the live-dead transcriptional reporter strain (GroEL:mCherry, tet:GFP) was grown in Difco Middlebrook 7H9 media supplemented with 10% OADC, 0.2% glycerol, 0.05% Tween-80 and Hygromycin B (50 ug/ml) to an OD of 0.6.

Monocyte-derived Macrophage Isolation—Human monocytes were isolated from human buffy coats using a standard Ficoll gradient and subsequent CD14+ cell positive selection (Stemcell Technologies, Tukwila, Wash.). Selected monocytes were cultured in low-adherence flasks (Corning) for 8 days with RPMI media (Invitrogen) supplemented with 10% heat inactivated FCS (Invitrogen) prior to infection with Mtb.

Mtb Infection—Mtb culture was pelleted and washed twice with RPMI+10% FCS and filtered through a 5 um syringe filter. MDM were infected at a multiplicity of infection (MOI) of 1:1. Cells were infected for four hours prior to washing twice with PBS and preparation for cell sorting. A sham population was performed in parallel (sham).

Macrophage Fluorescence Activated Cell Sortin—Mtb-infected macrophages were detached from low attachment plates using pre-warmed trypsin. Cells were pelleted and resuspended in FACS buffer (1×PBS, 2% FCS, 1 mM EDTA) and sorted on an Aria IIu. Two populations were isolated for single-cell RNA sequencing analysis. From the infected cells, approximately 500,000 GFP+ cells were sorted directly into cell culture media (RPMI+10% FCS). From the sham infection 500,000 cells were sorted directly into cell culture media (RPMI +10% FCS). Cells were reseeded into low adherence plates for an additional 20 hours prior to single cell RNA sequencing.

Single-Cell RNA-Sequencing—100,000 barcoded mRNA capture beads (ChemGenes, Macosko-2011-10) were loaded into the PDMS array, and the arrays were rocked back and forth to ensure optimal loading of beads into microwells as previously described. Once the beads had settled into wells, the arrays were repeatedly rinsed to remove excess beads on the surface of the arrays. The arrays were washed with 1×PBS and then 5 mL of RPM1+10% FCS. The media was then aspirated, followed immediately with dropwise addition of 1.5×10⁴ cells onto the surface of the array. After periodic gentle rocking and washing, a polycarbonate membrane (Sterlitech) was attached to the membrane surface.

After membrane attachment, the array was transferred to a dish containing lysis buffer for cell lysis in (5M GTCN, 1% 2-mercaptoethanol, 1 mM EDTA, and 0.1% Sarkosyl in 1×PBS, pH 6.0) and allowed to rock at room temperature for 20 minutes. The lysis buffer was then aspirated and arrays were washed once with 5 mL of hybridization buffer (2M NaCl, 1×PBS, 0.5% Tween20 (pH 7.5)). The hybridization buffer was aspirated and replaced with another 5 mL, followed by rocking for 40 minutes at room temperature. Beads were removed from microwell arrays by scraping the array with a microscope slide.

Recovered beads were transferred to a 1.5 mL tube and reverse transcription was performed using Maxima H-reverse transcriptase (Invitrogen). The beads were then incubated at room temperature for 30 minutes with end-over-end rotation, followed by 90 minutes at 50° C. with rotation. Beads were then treated with ExoI to digest unoccupied primer sites (NEB). Reaction was incubated at 37° C. for 45 minutes with end-over-end rotation.

Beads were washed and aliquoted into 2000 bead aliquots for PCR amplification using KAPA HiFi PCR Mastermix (KAPA Biosystems). The pooled PCR library was purified using Agencourt AMPure XP beads (Beckman Coulter) at a 0.6× volumetric ratio and quantified using Qubit Fluorometric Quantitation (Thermo Fisher)

Tagmentation was performed on a total of 800 pg of pooled cDNA library. Tagmented and amplified sequences were purified using Agencourt AMPure XP beads at a 0.6× volumetric ratio and quantified using Qubit Fluorometric Quantitation. The size distribution of purified sequencing libraries was determined using the Agilent D1000 Screen Tape System (Agilent Genomics). The average fragment size of sequenced libraries was between 400 and 700 base pairs in length. Seq-Well libraries were all sequenced on an Illumina NextSeq500 at a final concentration of 2.4 pM. 20 bases were allocated (12 bp cell barcode and 8 bp UMI) for Read 1, which was primed using Custom Read 1 Primer.

Transcriptome Alignment—Read alignment was performed as in Macosko et al., Cell, 2015. Briefly, for each NextSeq sequencing run, raw sequencing data was converted to FASTQ files using bcl2fastq2 that were demultiplexed by Nextera N700 indices corresponding to individual samples. Reads were first aligned to both HgRC19 and mm10, and individual reads were tagged according to the 12-bp barcode sequence and the 8-bp UMI contained in read 1 of each fragment. Following alignment reads were binned and collapsed onto 12-bp cell barcodes that correspond to individual beads using DropSeq tools (mccarrolllab.com/dropseq/). Barcodes were collapsed with a single-base error tolerance (Hamming distance=1) with additional provision for single insertions or deletions. An identical collapsing scheme (Hamming distance=1) was then applied to unique molecular identifiers to obtain quantitative counts of individual mRNA molecules.

Data Normalization—Digital gene expression matrices were obtained by collapsing filtered and mapped reads by 8-bp unique molecular identifier sequence within each cell barcode. From each cell, we performed library-size normalization UMI-collapsed gene expression values for each cell barcode were scaled by the total number of transcripts and multiplied by 10,000. Scaled expression data were then natural-log transformed prior to analysis using Seurat.

Data Analysis—Following sequence alignment, we identified a total of 12,000 infected and 22,000 uninfected cells with greater than 1,000 mapped transcripts. Applicant focused on a subset of macrophages with greater than 5,000 detected transcripts (Infected: 2,053 cells, Uninfected: 443 cells). Cells with greater than 4% mitochondrial RNA transcripts were omitted from subsequent analysis. Principal components analysis was performed among a set of variables genes, defined by genes with log mean expression greater than 0.0125 and dispersion (variance/mean) greater than 0.0125. Applicant performed t-SNE clustering using the first 15 principal components. Applicant identified 7 distinct clusters of cells in the t-SNE plot using the FindClusters function in Seurat with resolution=1.1. Applicant analyzed cluster-defining genesets using Ingenuity Pathway Analysis, ClueGO, and Enrichr to identify transcriptional programs or transcription factors significantly enriched in a particular gene set.

Flow Cytometry Analysis—Uninfected macrophages were blocked with anti-mouse FcR antibody (CD16/CD32, Biolegend) for 15 min at room temperature in FACS buffer (PBS with 2% FCS and 1 mM EDTA). Subsequently, cells were surface stained with appropriate antibody panels CD36-APC (Biolegend) or CD52-PE/Cy7 (Biolegend) at room temperature. Cells were washed three times with FACS buffer prior to analysis on Aria IIu.

GFP:Mtb infected macrophages were blocked with anti-mouse FcR antibody (CD16/CD32, BioLegend) for 15 min at room temperature in FACS buffer (PBS with 2% FCS and 1 mM EDTA). Subsequently, cells were surface stained with appropriate antibody panels SLAMF7-PE/Cy7 (Biolegend) or IFNGR1-APC (Miltenyi) at room temperature. Cells were washed three times with FACS buffer prior to analysis on Aria IIu.

Confocal Microscopy—Mtb-infected macrophages were fixed for 2 hr with 2% PFA at room temperature. After fixation, the samples were blocked in 0.3% Triton-X 100/5% donkey serum serum/1×PBS for 60 min at RT, washed, and incubated with anti-p53 (Cell Signaling Technology Clone DO-7; 1:100) overnight at 4° C. The samples were washed again and stained with a Alexa Fluor 647-conjugated donkey anti-mouse secondary antibody (Thermo Fisher; 1:1000) for 1 hr at RT. Images were obtained using a Zeiss LSM 510 Confocal Microscope fitted with a 20× objective.

Live Dead Imaging—Macrophages were plated in a 96-well plate and infected with the transcriptional reporter Mtb strain at an MOI of 1:1. After four hours, cells were washed to remove extracellular bacteria and then replenished with media containing DMSO, nutlin-3a, PS-1145, or BMS-34551 (Sigma Aldrich). Infection proceeded for 3 days prior to the addition of 200 ng/mL anhydrotetracycline to each well to induce transcriptionally viable cells to express GFP. On day 5, cells were fixed for 2 hr with 2% PFA at room temperature. Images were obtained via Operetta High-Content Imaging Fluorescence Microscope (Perkin-Elmer) outfitted with 20×NA objective. Total Mtb bacterial burden was determined based on mCherry+ pixels. Transcriptionally active Mtb bacterial burden was determined based on GFP+ pixels. Between 1.5×10⁴ and 4×10⁴ cells per condition over technical triplicates per donor were analyzed using Columbus (Perkin-Elmer). Bacterial survival was calculated as a ratio of live to total bacteria (the number of GFP+ pixels (live) divided by the number of mCherry+ pixels (total burden).

### REFERENCES

1. Rohde, K. H., Veiga, D. F. T., Caldwell, S., Balázsi, G. & Russell, D. G. Linking the Transcriptional Profiles and the Physiological States of Mycobacterium tuberculosis during an Extended Intracellular Infection. PLOS Pathog. 8, e1002769 (2012).
2. Xue, J. et al. Transcriptome-Based Network Analysis Reveals a Spectrum Model of Human Macrophage Activation. Immunity 40, 274-288 (2014).
3. Shalek, A. K. et al. Single-cell RNA-seq reveals dynamic paracrine control of cellular variation. Nature 510, 363-369 (2014).
4. Lane, K. et al. Measuring Signaling and RNA-Seq in the Same Cell Links Gene Expression to Dynamic Patterns of NF-κB Activation. Cell Syst. 4, 458-469.e5 (2017).
5. Regot, S., Hughey, J. J., Bajar, B. T., Carrasco, S. & Covert, M. W. High-Sensitivity Measurements of Multiple Kinase Activities in Live Single Cells. Cell 157, 1724-1734 (2014).
6. Gierahn, T. M. et al. Seq-Well: portable, low-cost RNA sequencing of single cells at high throughput. Nat. Methods 14, 395-398 (2017).
7. Silverstein, R. L. & Febbraio, M. CD36, a Scavenger Receptor Involved in Immunity, Metabolism, Angiogenesis, and Behavior. Sci Signal 2, re3-re3 (2009).
8. Baitsch, D. et al. Apolipoprotein E induces antiinflammatory phenotype in macrophages. Arterioscler. Thromb. Vasc. Biol. 31, 1160-1168 (2011).
9. Koch, A. E. et al. Interleukin-8 as a macrophage-derived mediator of angiogenesis. Science 258, 1798-1801 (1992).
10. Kawai, T. & Akira, S. Signaling to NF-kappaB by Toll-like receptors. Trends Mol. Med. 13, 460-469 (2007).
11. Stanley, S. A., Johndrow, J. E., Manzanillo, P. & Cox, J. S. The Type I IFN Response to Infection with Mycobacterium tuberculosis Requires ESX-1-Mediated Secretion and Contributes to Pathogenesis. J. Immunol. 178, 3143-3152 (2007).
12. Novikov, A. et al. Mycobacterium tuberculosis triggers host type I interferon signaling to regulate IL-1β production in human macrophages. J. Immunol. Baltim. Md 1950 187, 2540-2547 (2011).
13. Kelly, B. & O'Neill, L. A. Metabolic reprogramming in macrophages and dendritic cells in innate immunity. Cell Res. 25, 771-784 (2015).
14. Gottschalk, R. A. et al. Distinct NF-κB and MAPK Activation Thresholds Uncouple Steady-State Microbe Sensing from Anti-pathogen Inflammatory Responses. Cell Syst. 2, 378-390 (2016).
15. Hughey, J. J., Gutschow, M. V., Bajar, B. T. & Covert, M. W. Single-cell variation leads to population invariance in NF-κB signaling dynamics. Mol. Biol. Cell 26, 583-590 (2015).
16. Kellogg, R. A., Tian, C., Etzrodt, M. & Tay, S. Cellular Decision Making by Non-Integrative Processing of TLR Inputs. Cell Rep. 19, 125-135 (2017).
17. Liu, P. T. et al. Toll-Like Receptor Triggering of a Vitamin D-Mediated Human Antimicrobial Response. Science 311, 1770-1773 (2006).
18. Yamada, H., Mizuno, S., Reza-Gholizadeh, M. & Sugawara, I. Relative Importance of NF-κB p50 in Mycobacterial Infection. Infect. Immun. 69, 7100-7105 (2001).
19. Madenspacher, J. H. et al. p53 integrates host defense and cell fate during bacterial pneumonia. J. Exp. Med. 210, 891-904 (2013).
20. Menendez, D. et al. The Toll-Like Receptor Gene Family Is Integrated into Human DNA Damage and p53 Networks. PLOS Genet. 7, e1001360 (2011).
21. Menendez, D., Shatz, M. & Resnick, M. A. Interactions between the tumor suppressor p53 and immune responses. Curr. Opin. Oncol. 25, 85-92 (2013).
22. Cruz, A. et al. IL-17A Promotes Intracellular Growth of Mycobacterium by Inhibiting Apoptosis of Infected Macrophages. Front. Immunol. 6, (2015).
23. Vassilev, L. T. et al. In Vivo Activation of the p53 Pathway by Small-Molecule Antagonists of MDM2. Science 303, 844-848 (2004).
24. Chène, P. Inhibiting the p53-MDM2 interaction: an important target for cancer therapy. Nat. Rev. Cancer 3, 102-109 (2003).
25. Paek, A. L., Liu, J. C., Loewer, A., Forrester, W. C. & Lahav, G. Cell-to-Cell Variation in p53 Dynamics Leads to Fractional Killing. Cell 165, 631-642 (2016).
26. Kaufmann, E. et al. BCG Educates Hematopoietic Stem Cells to Generate Protective Innate Immunity against Tuberculosis. Cell 172, 176-190.e19 (2018).

### Example 2

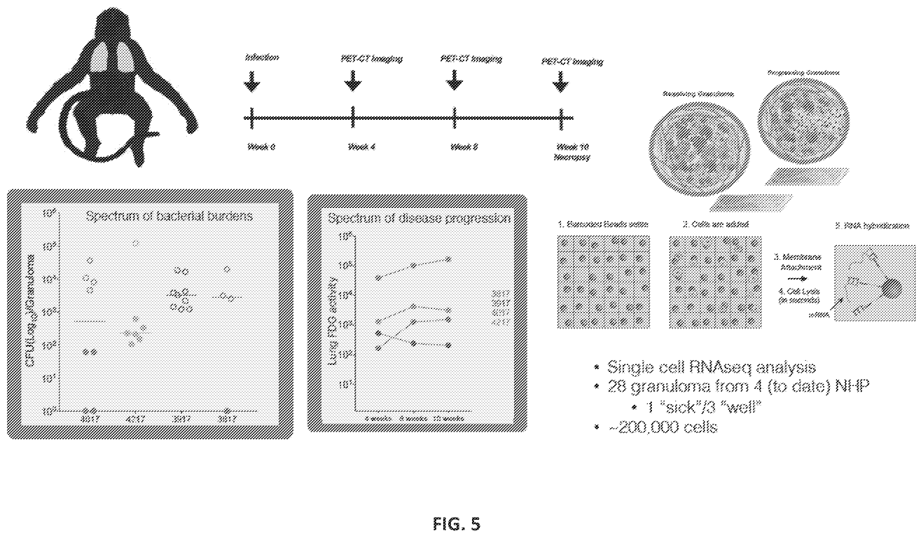

FIG. 5 shows an exemplary method for unbiased definition of the features of restrictive and permissive granulomas. Single cell RNAseq was performed on cells from granulomas were performed and the sequences were analyzed. In brief, barcoded beads were settled on a chip. About 200,000 cells from 28 granuloma from 4 non-human primates (NHP) were added to the chip. The 4 non-human primates were infected by MTB. Among the 4, 1 had restrictive granulomas and 3 had permissive granulomas. After the cell were added, membrane was attached and the cells were lysed (in seconds). mRNA hybridization was performed and the nucleic acids molecules were sequenced and analyzed using single cell RNAseq technology. Single cell RNAseq may be performed according to methods described in Gierahn et al., Nature Methods volume 14, pages 395-398 (2017). The sequencing and analysis may also be performed with other methods, e.g., scpTCR sequencing.

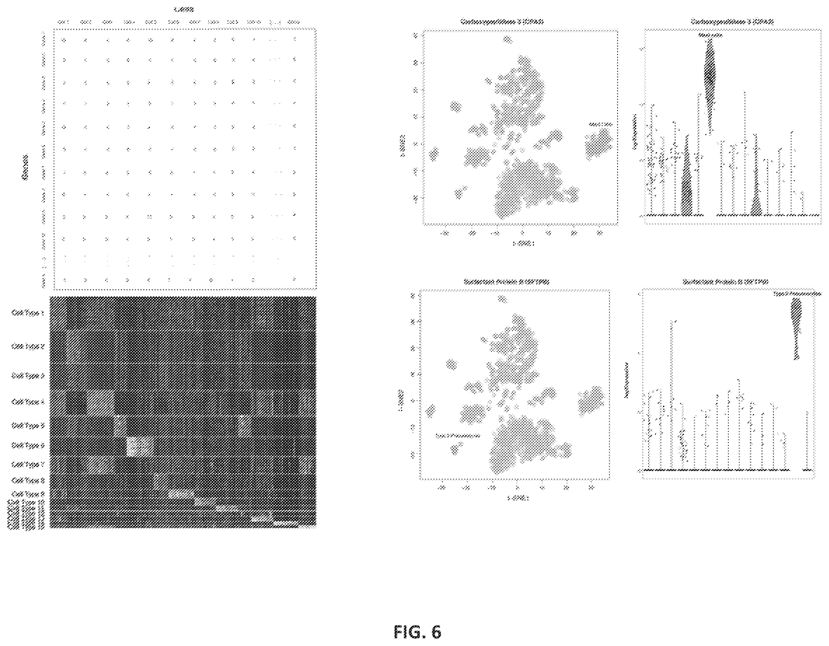

FIG. 6 shows analysis of the single cell RNAseq results. The analysis comprises data-driven cell identification, including identification and analysis of differentially expressed genes, hierarchical clustering, excluding clusters only found in a single granuloma, Principal Component Analysis (PCA) and dimensionality reduction, and enrichment based on cell type identification.

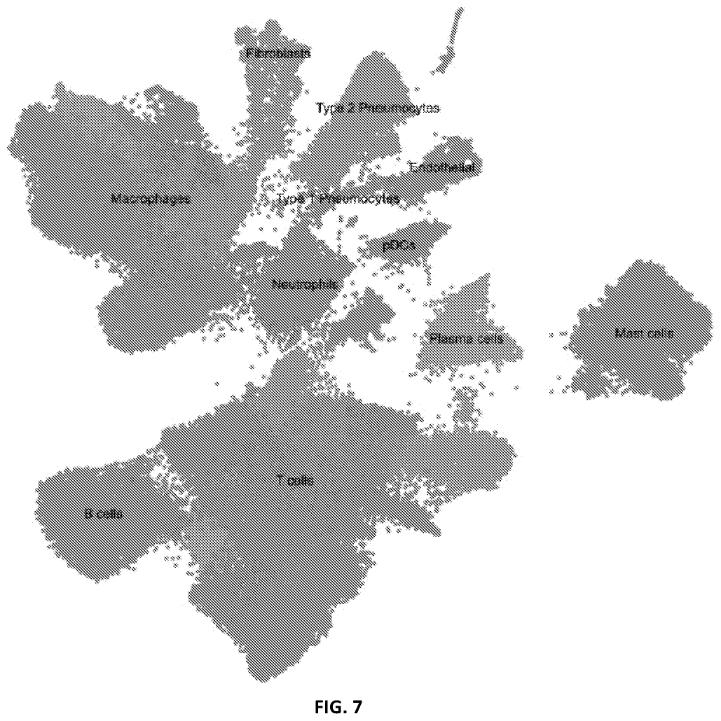

FIG. 7 shows Tuberculosis (TB) granuloma atlas. About 180,000 cells in 26 granulomas from 4 non-human primates were analyzed.

FIG. 8 shows identification of T cell clusters in NHP with early progression in disease.

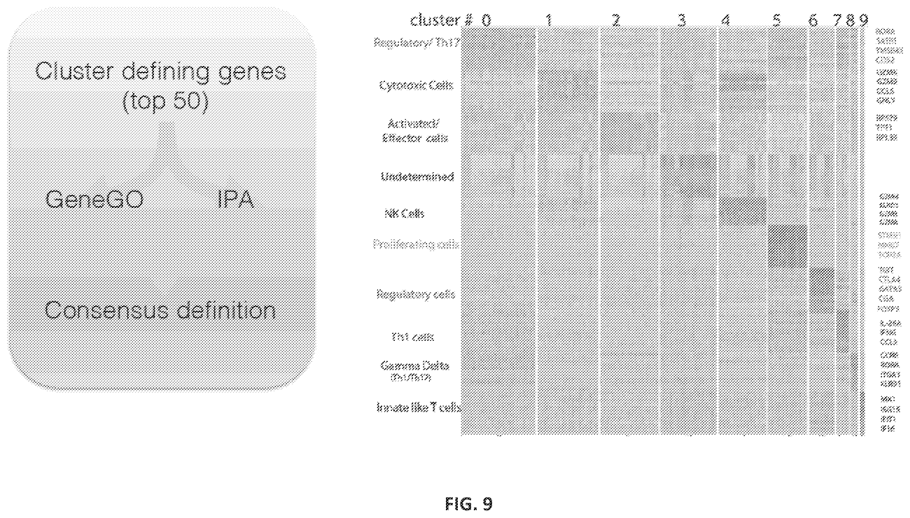

FIG. 9 shows cell type identification.

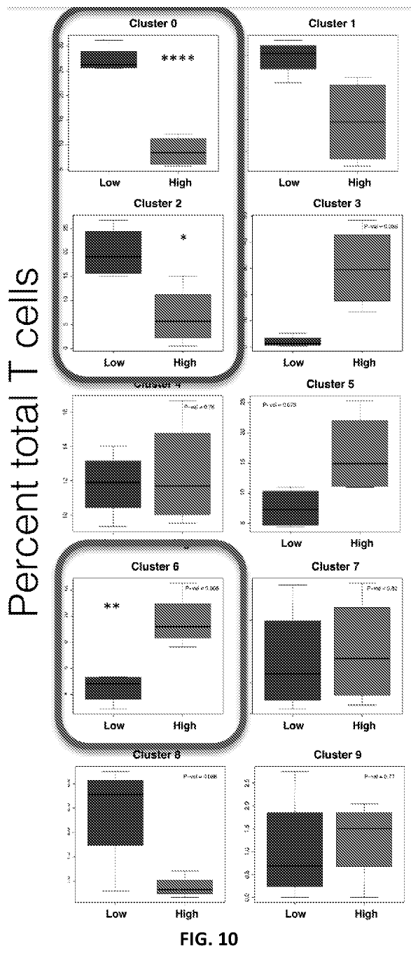

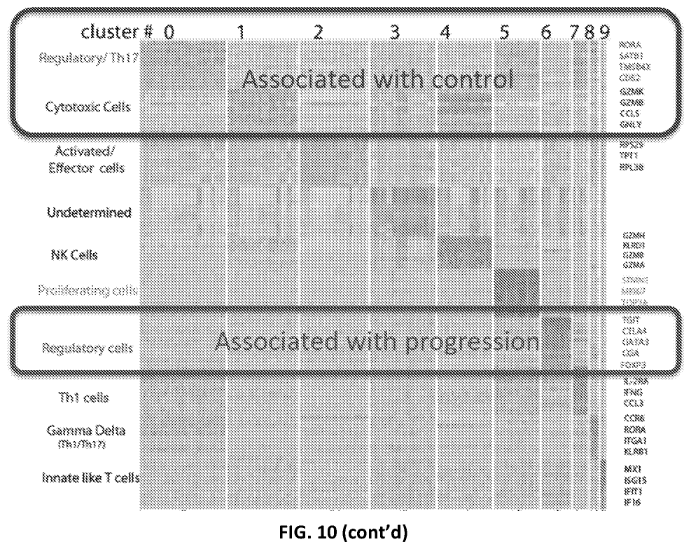

FIG. 10 shows identification of T cell clusters and correlation with control in an NHP with early progression.

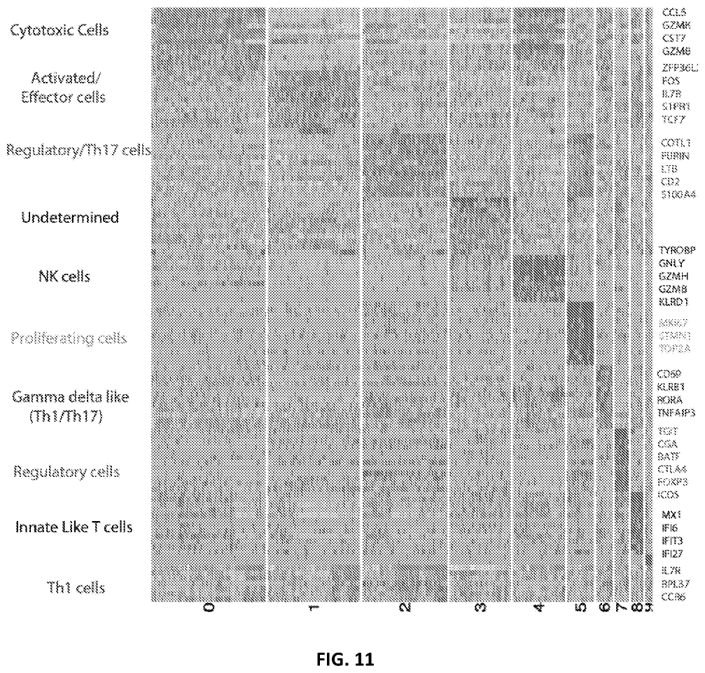

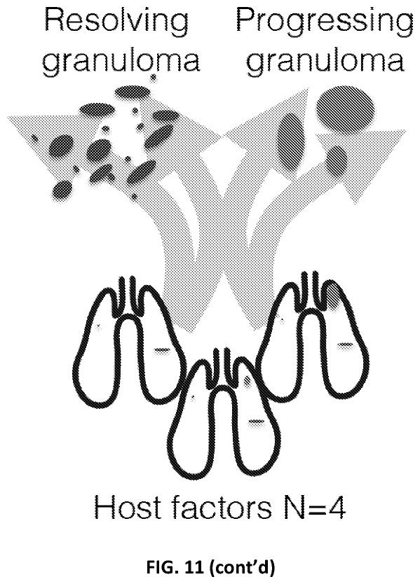

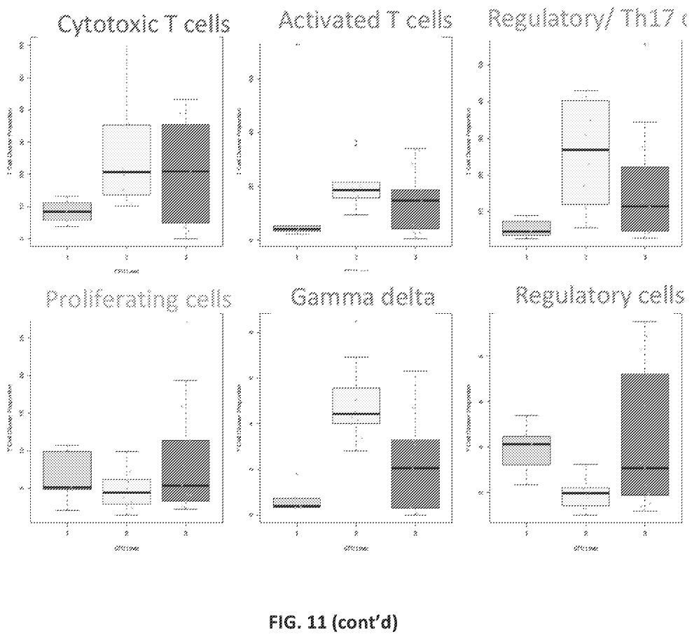

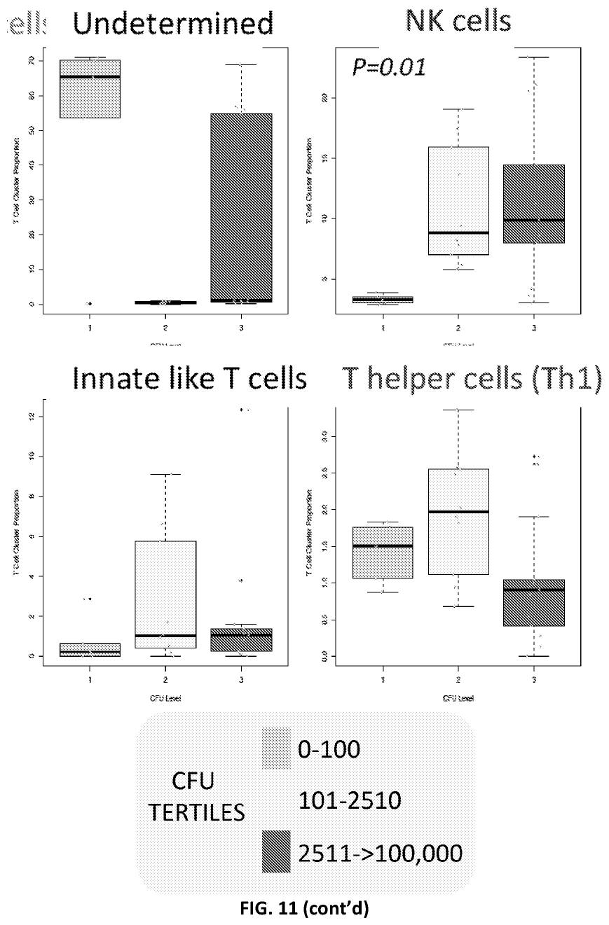

FIG. 11 shows identification of T cell clusters and correlation with control in another NHP with early progression. The results suggest that T cell subsets may be contextual, i.e., correlation with granulomas progressiveness may not be generalized across individual hosts.

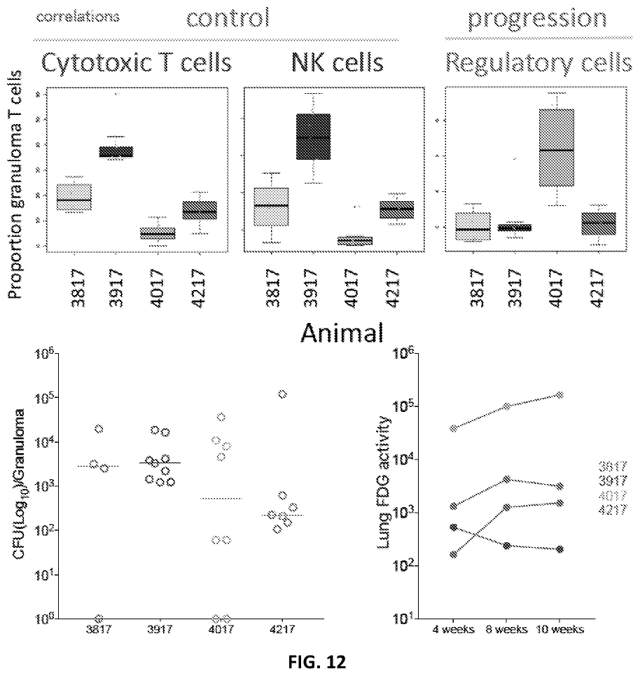

FIG. 12 shows composition of T cell compartment varies across hosts.

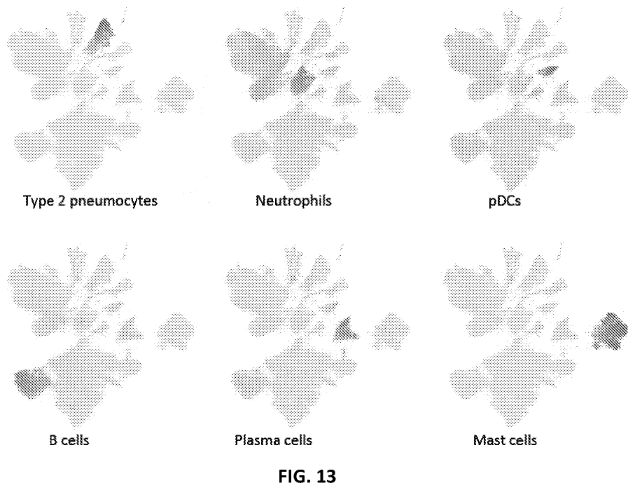

FIG. 13 shows exploration of cell populations that had correlation with progressiveness shared across hosts.

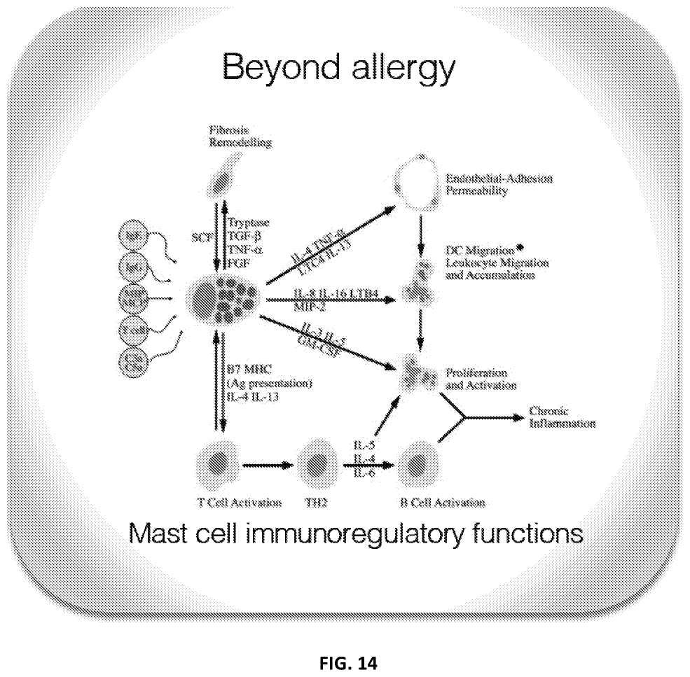

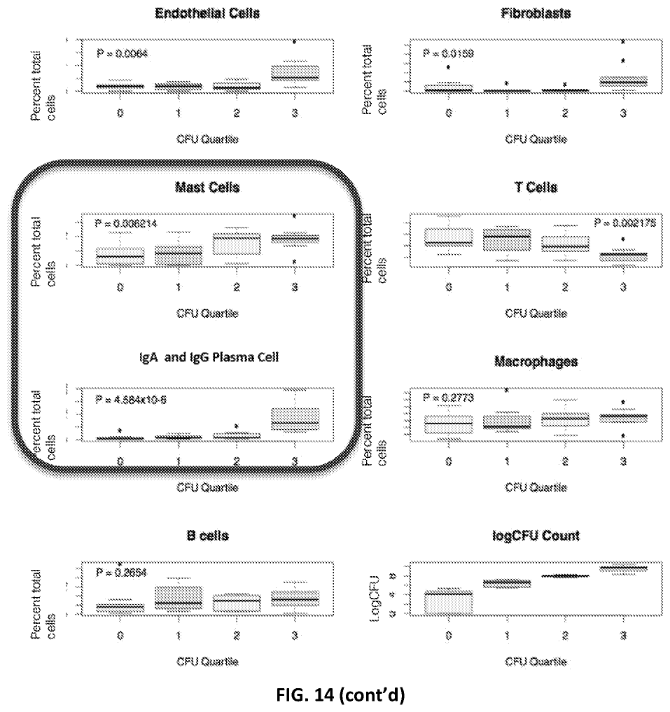

FIG. 14 shows strong correlation in all animals between progressiveness and mast cells or plasmablasts.

### Example 3—Cellular Ecology of M. tuberculosis Granulomas Identifies Cellular and Molecular Correlates of Bacterial Control

Mycobacterium tuberculosis (MTB) infection is the leading cause of death from infectious disease around the globe. Each year an estimated 1.5 million people die from complications of MTB infection, while an estimated ⅓ of the world's population is latently infected. Critically, the development a safe and effective vaccine for MTB infection is limited by an incomplete understanding of immunologic control in the setting of natural infection.

Here, Applicant presents the findings that using high-throughput single-cell genomic profiling with a non-human primate (NHP, M. fascicularis) model of MTB infection that most closely recapitulates the primary features of human disease. In this model, Applicant observed a spectrum of disease across animals that includes both latent and active disease. In both latent and active disease, granulomas simultaneously existed with variable tolerance to bacterial replication within the same animal—some lesions spontaneously resolved, while others supported active bacterial replication. In the present study, a total of 4 cynomolgus macaques were bronchoscopically infected with a low-dose of MTB, and the development of granulomas was tracked using serial PET-CT imaging. In total we harvested a total of 28 granulomas that spanned a wide range of bacterial burdens, which allowed to relate granuloma composition and cellular phenotypes to granuloma-level bacterial burden. Importantly, Applicant had sampled lesions at 10 weeks post-infection, a time-point in which instantaneous bacterial burden reflected immunologic control of infection.

In this model, Applicant performed high-throughput single-cell mRNA sequencing across 28 granulomas and generated 113,846 high-quality single-cell transcriptomes. After robust quality control, Applicant identified 13 primary cell types from lymphoid, myeloid, and stromal lineages in MTB granulomas. Applicant observed considerable heterogeneity among immune cell populations, and through directed analysis we identified multiple populations of macrophages and T cells. Applicant leveraged granuloma-level metadata including end-point colony forming units (CFU), bacterial chromosomal equivalents (CEQ), serial PET-CT imaging, and bacterial barcode sequencing to identify immune populations correlated with granuloma-level bacterial burden.

The key findings and contributions include:

Applicant performed high-throughput single-cell mRNA sequencing in a non-human primate model of TB infection that was the closest model of human infection. Across 4 animals, Applicant obtained a collection of 28 granulomas that spanned a broad range of bacterial burden that enabled interrogation of the relationship between immune populations and bacterial control.

Applicant identified immune correlates of MTB control at the level of individual granulomas. In aggregate, the abundance of T cells was strongly associated with reduced bacterial burden. Applicant further identified unexpected associations between mast cells and plasma cells and increased bacterial burden.

Applicant identified T cell expression patterns that segregated with granuloma-level bacterial burden. Applicant identified a population of Th1-Th17 cells that most strongly associated with MTB control across granulomas. Importantly, this T cell population was characterized by expression of genes previous associated with Th17 effector function and bore striking similarities to recently described T cell populations that protected against pulmonary bacterial infection. Applicant also observed increases in effector CD8 T cells in low-burden lesions.

Applicant generated high-throughput TCR reconstruction and describe patterns of clonal expansion across granulomas. In this analysis, Applicant observed patterns of clonal expansion between high and low burden lesions, along with significant clonal sharing across the spectrum of bacterial burden. Applicant charted the distribution of T cell phenotypes across expanded clones, and examined the presence of convergent clonal selection within the T cell pool.

The data revealed a nuanced relationship between timing of granuloma formation and bacterial control. Among lesions observed only at 10-weeks post-infection, Applicant observed the lowest bacterial burdens, despite similar cumulative bacterial burdens. In comparison of early-forming and late-blooming lesions, Applicant observed similar associations with Th1-Th17 cells and effector CD8 T cells. However, among early-forming lesions, Applicant observed burden association with effector CD8 T cells. These data suggest that early killing by CD8 T cells and late-onset adaptive Th1-Th17 differentiation represented distinct paths to bacterial control.

Applicant identified broad patterns of granuloma composition across granulomas that distinguished high and low burden lesions, and Applicant reported coordinated expansion of T cell populations in low-burden lesions, while high-burden lesions were show increases in stromal and myeloid populations.

Finally, Applicant generated cell-cell interaction networks across granulomas and examine the relationship between receptor-ligand networks and bacterial burden. In high burden, lesions we observed increased interactions between myeloid and stromal populations, while we observe upregulation of signaling between T cells and macrophages. Specifically, among high burden lesions Applicant observed increased IL-13 signaling from mast cells that broadly interacts with macrophages and T cells. In low-burden lesions, Applicant observed upregulation of signaling involving IFNG, CCL3, and CCL5.

In summary, Applicant identified a number of immune populations correlated with bacterial burden. The data revealed a nuanced relationship between the timing of granuloma formation and bacterial control. Collectively, the data nominated putative cellular targets for protective vaccination as well as targets for host-directed therapy in MTB infection.

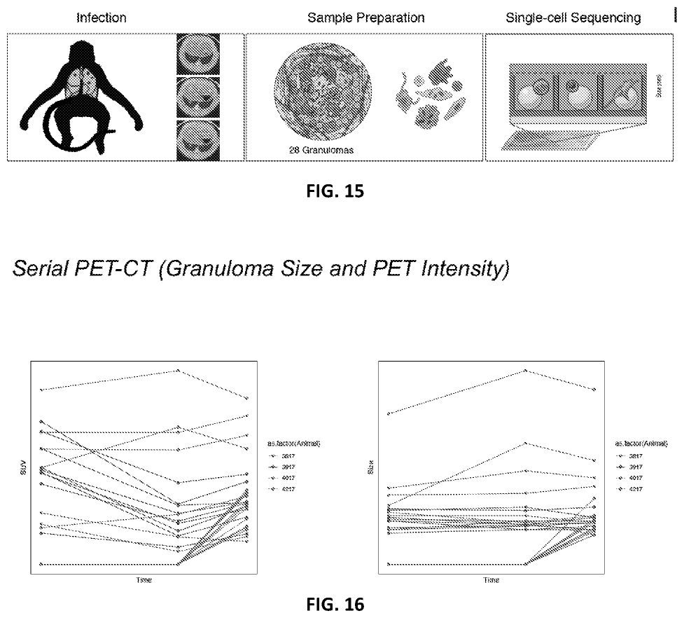

Using a non-human primate model of M. tuberculosis (MTB) infection, Applicant performed high-throughput single-cell mRNA sequencing to identify cellular and molecular features of bacterial control within MTB granulomas (FIG. 15). In total, Applicant obtained 113,864 cells from 28 granulomas across 4 cynomolgus macaques infected with MTB. Applicant performed analysis of single-cell transcriptomes to identify trends in cell type composition and cellular phenotypes that distinguish resolving and non-resolving granulomas. Applicant observed expansion of T cells in lesions with lower bacterial burden and an unexpected elevation in the frequency of mast and plasma cells among high burden lesions. Among T cells, Applicant observed an increase in the frequency of Th1-Th17 cells and effector CD8 T cells in low burden lesions. Applicant performed integrated TCR reconstruction and chart the relationship between clonal expansion, T cell phenotype and bacterial burden. Applicant further identified coordinated shifts in granuloma composition and generated cell-cell interaction networks that uncovered putative receptor-ligand axes that might underlie bacterial control. Finally, Applicant observed that timing of granuloma formation influenced the ability of lesions to control bacterial replication. Collectively, the data suggest a nuanced picture of bacterial control in which lesions that formed in the initial stages of infection create an environment that supported bacterial replication, while late-blooming lesions were better able to control bacteria following the onset of adaptive immunity.

Introduction

Mycobacterium tuberculosis (MTB) is the causative agent of human tuberculosis infection, which results in 1.3 million deaths each year globally and latently infects an estimated one third of the world's population (citation). Currently, there is no highly effective vaccine for the prevention of MTB infection, and the emergence of bacterial resistance has significantly diminished the effectiveness of mainline treatments for MTB infection (citation). Despite the prevalence and impact of the disease, a complete understanding of the host immune response at the minimal unite of infection—the granuloma is needed. Upon infection of alveolar macrophages, M. tuberculosis is sequestered in multicellular aggregates of stromal and immune cells known as granulomas.

The presence of numerous macrophages infected with MTB is a hallmark of MTB granulomas, but a comprehensive understanding of how overall cellular composition and macrophage phenotypes synergize to influence bacterial control remains elusive. Based on immunohistochemistry, a number of macrophage simultaneously existed within the granuloma micro-environment including epithelioid macrophages, foamy macrophages, and multi-nucleated giant cells. MTB granulomas were characterized by abundant lipid-laden macrophages that surround central regions of caseous necrosis.

While innate responses dominate the early stages of infection, T cell-mediated immunity is an essential feature of the host response and control of MTB infection. Ablation of instructive T cell cytokines results in profound loss of immunologic control in mice. The identity and distribution of T cell phenotypes dictates bacterial control in MTB infection. Increases in regulatory T cells, which act to limit inflammatory responses, are broadly associated with increases in bacterial burden and delayed onset of effector T cell responses. Effector T cell responses in MTB infection generally arise in response to migration of bacteria to a draining lymph node.

In this model, Applicant observed a spectrum of disease across animals that included both latent and active disease. In both latent and active disease, granulomas simultaneously existed with variable tolerance to bacterial replication within the same animal—some lesions spontaneously resolved, while others supported active bacterial replication. In this view, individual MTB granulomas was seen as distinct micro-environments whose fates were determined by local immune dynamics. Granulomas that formed in the context of pre-existing MTB infection readily eliminated bacteria upon secondary challenge. The cynomolgus macaque model of MTB infection most closely recapitulated the essential features of human tuberculosis infection in which a similar spectrum of latent and active disease MTB granulomas from a single individual display variable ability to eliminate bacteria.

Results

Dynamics of M. tuberculosis Infection in Non-Human Primates

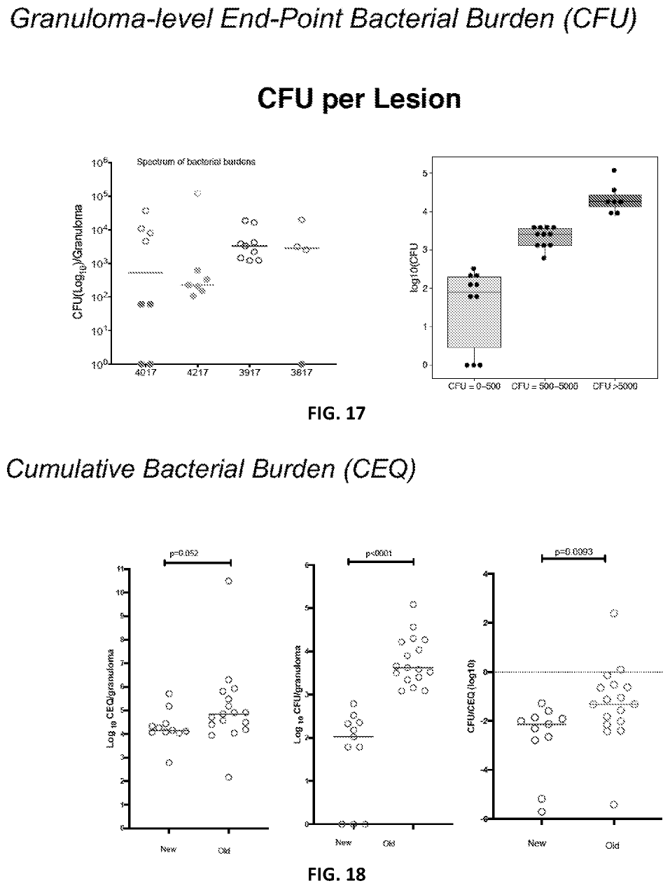

A total of 4 cynomolgus macaques were bronchoscopically infected with a low-dose inoculum of M. tuberculosis (Methods). Serial PET-CT imaging was performed to record the size, PET intensity and timing of granuloma formation for pulmonary granulomas at 4, 8 and 10 weeks post-infection (FIG. 16). On PET-CT imaging, Applicant observed lesions on scans at all timepoints, while Applicant observed other lesions only on 10-week pre-necropsy scans. Following harvest of granulomas at necropsy, granuloma-level bacterial burden was obtained through serial CFU assays (FIG. 17, Methods). Importantly, Applicant observed a wide range of bacterial burden across granulomas included in sequencing analyses (CFU Range: 0-120,600). Applicant further determined the cumulative bacterial burden for each granuloma by sequencing-based determination of bacterial chromosomal equivalents (CEQ, FIG. 18). Comparison of the cumulative bacterial burden (CEQ) to the end-point burden (CFU) provides a metric that reflects the extent of bacterial killing over time (CFU/CEQ ratio). Applicant are further able to reconstruct the dynamics of infection (i.e. original and spreading lesions) by sequencing of bacterial barcodes (Methods).

Single-Cell mRNA Sequencing of MTB Granulomas

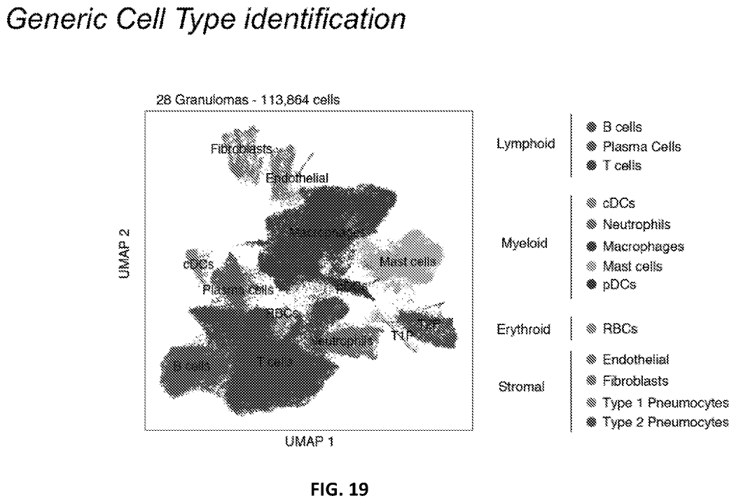

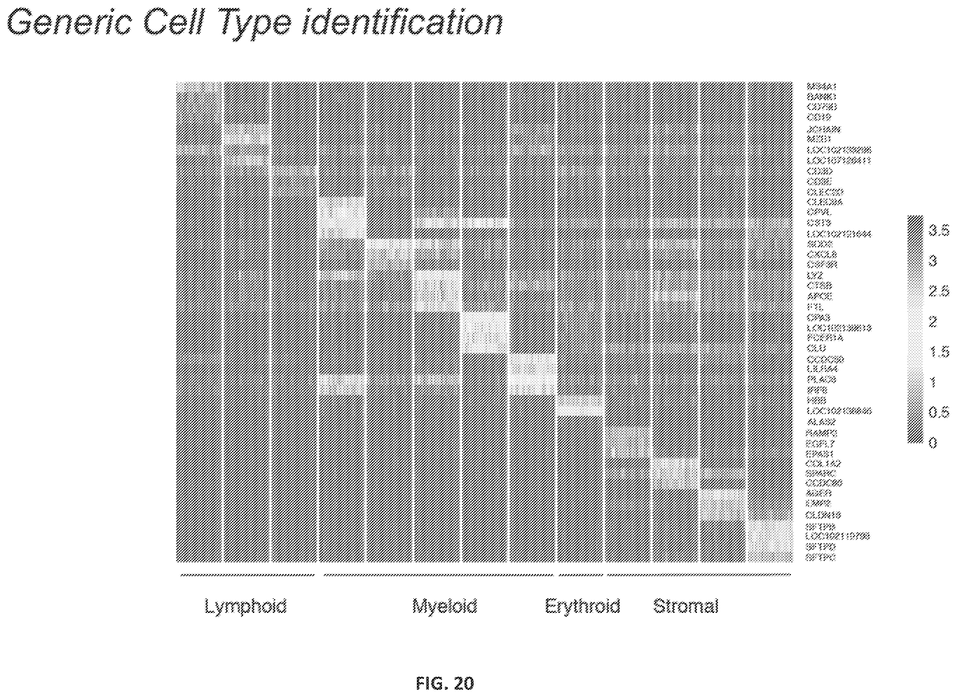

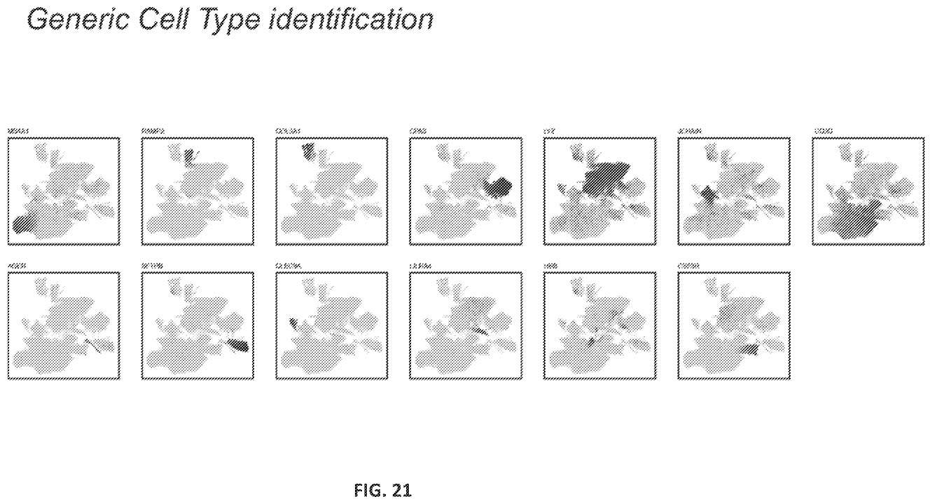

Generic Cell Type Identification: To identify the cellular and molecular correlates of bacterial control, Applicant performed high-throughput single-cell sequencing across a subset of 28 granulomas obtained from 4 cynomolgus macaques infected with M. tuberculosis using the Seq-Well platform. Individual granulomas were resected from the lungs of infected animals at 10-weeks post-infection, and a single-cell suspension was obtained for each lesion and applied to Seq-Well device (Methods). After robust technical correction, Applicant performed downstream analysis on 113,864 high-quality single-cell transcriptomes (Methods). Single-cell transcriptional profiles separated into clusters which were assigned a generic cell type identity using a combination of computational classification and manual curation (Methods). In total, Applicant identified 13 generic cell types, with numerous sub-clusters, particularly among T cells and macrophages (FIG. 19). Specifically, Applicant identified B cells (CD79A and BANK1), conventional dendritic cells (cDCs, CLEC9A), plasmacytoid dendritic cells (pDCs, LILRA4), endothelial cells (CD93), erythrocytes (HBB), fibroblasts (COL1A1), macrophages (LYZ), mast cells (CPA3), neutrophils (CSF3R and PLEK), plasma cells (JCHAIN), T cells (CD3D and IL7R), Type 1 Pneumocytes (AGER) and Type 2 Pneumocytes (SFTPB and SFTPC) (FIG. 20). Importantly, assigned cell-type classifications closely corresponded to those observed in hierarchical clustering (FIG. 20; Methods). For each generic cell type, Applicant observed exclusive enrichment of the cell-type gene expression signature with each cluster (FIG. 21). Applicant further performed extensive validation of cell-type assignments through comparison to reference expression signatures contained in the Savant database (Methods).

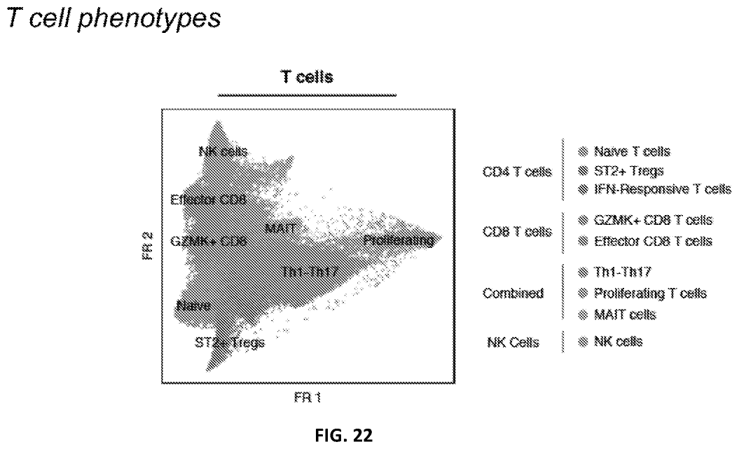

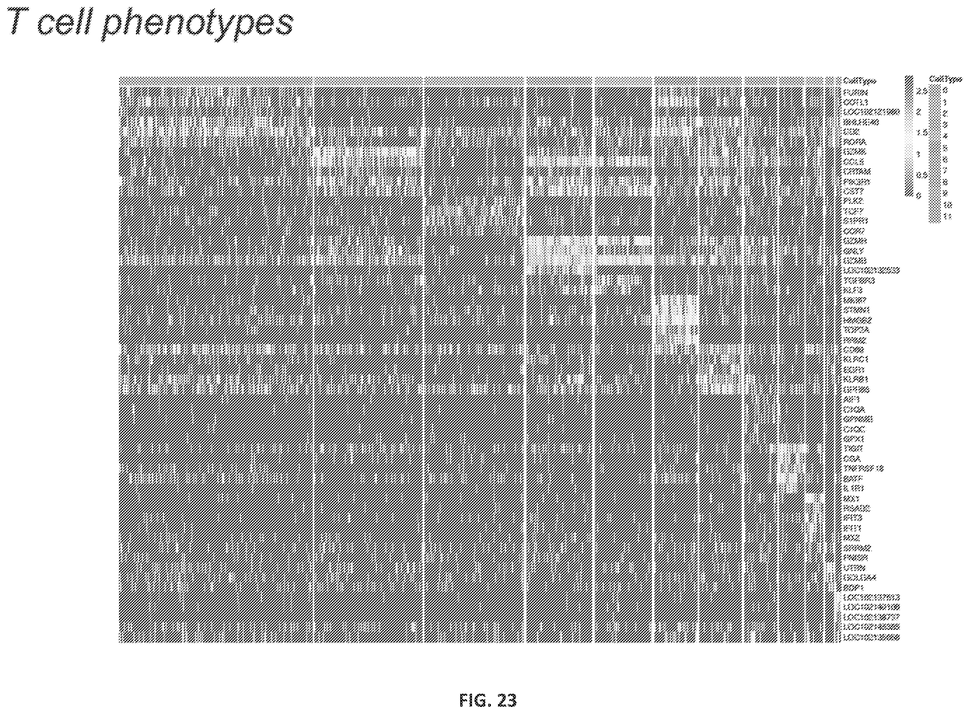

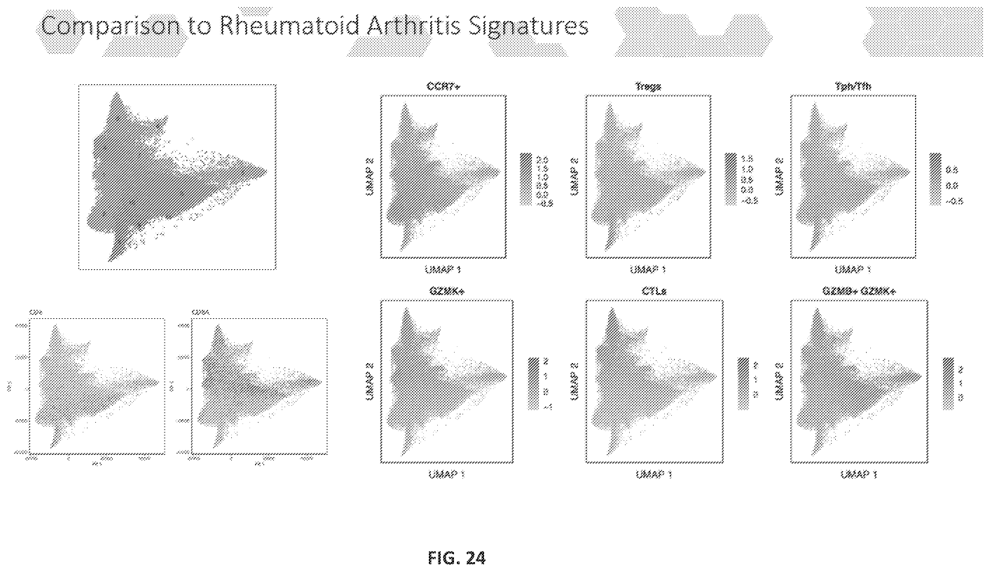

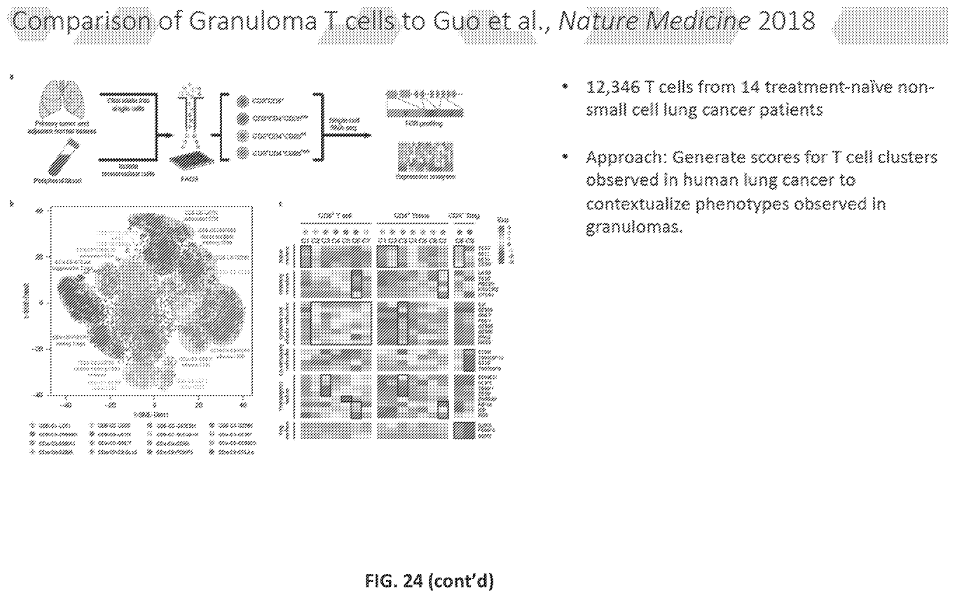

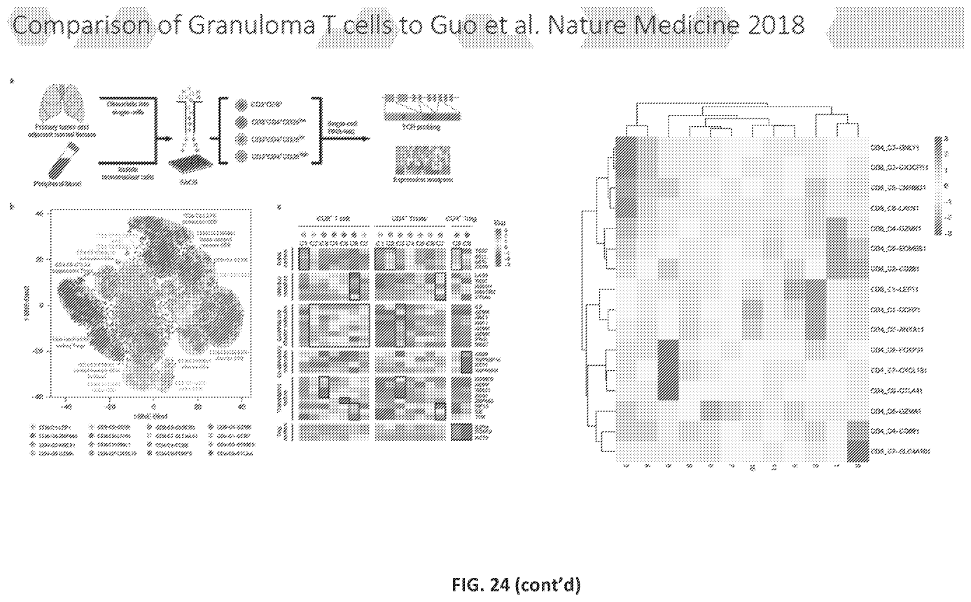

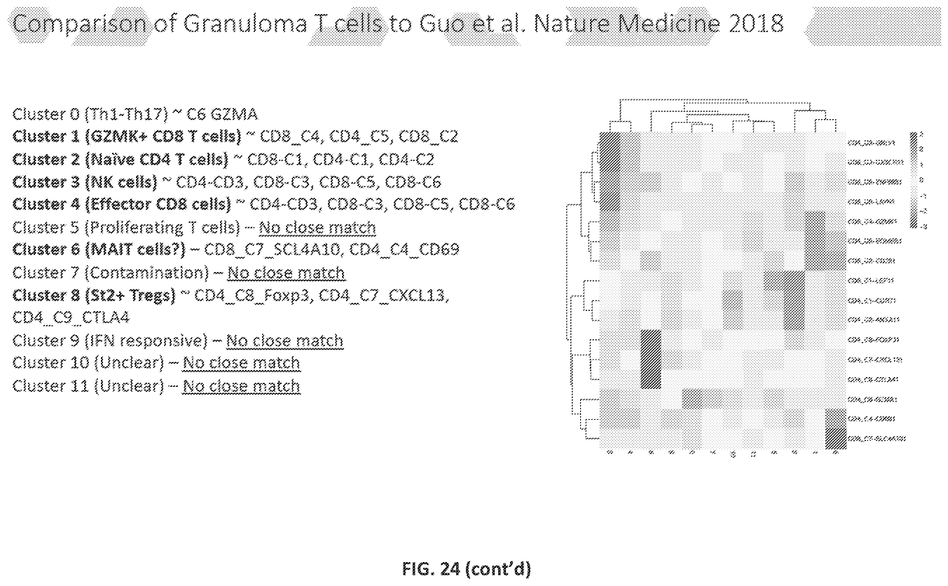

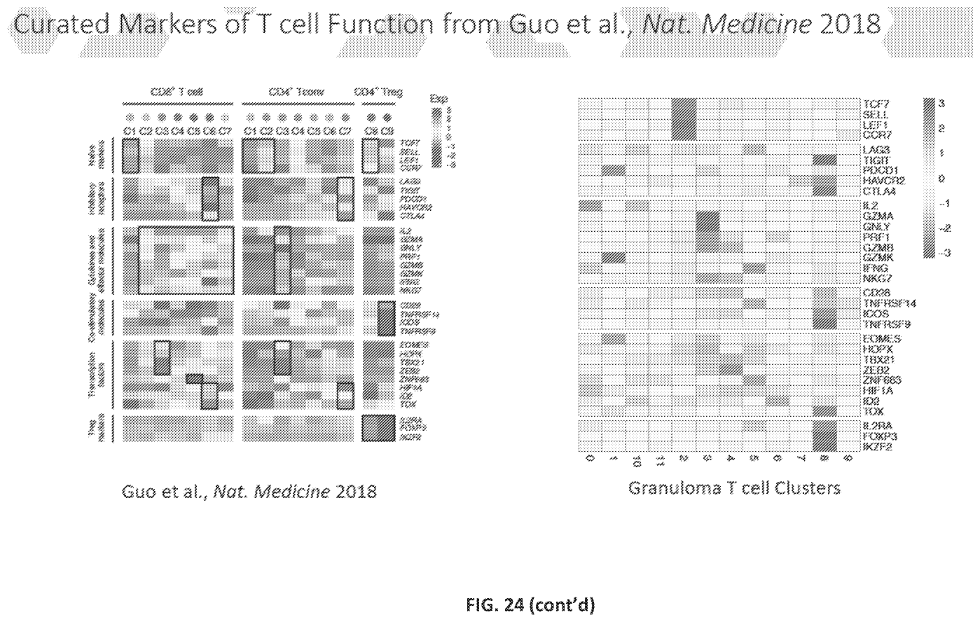

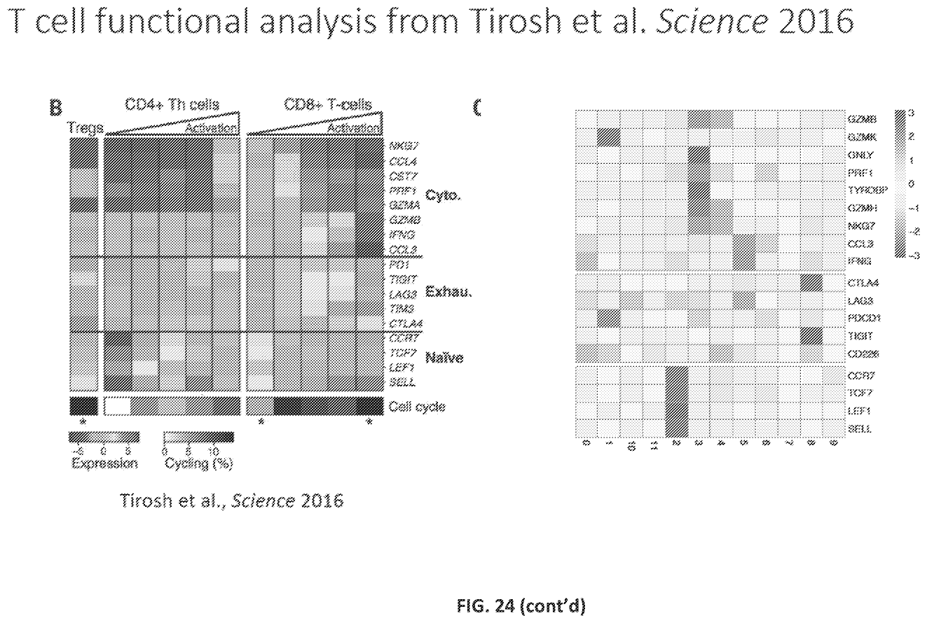

T cell population diversity: To identify granular correlates of immune protection in MTB granulomas, Applicant performed sub-clustering analysis among T cells and macrophages. Applicant performed separate dimensionality reduction among 46,460 T cells and identify 12 sub-clusters of T cells across 28 granulomas (FIG. 22). Applicant detected a population of NK cells that was distinguished by the highest expression of cytotoxic effector molecules (GZMA, GZMB, GNLY, and PRF1) and the lowest detection of TCR gene expression. Applicant identify 3 clusters that consistent of primarily CD4 expressing T cells. Specifically, Applicant identify a population of Naïve T cells (CCR7, LEF1, and SELL), regulatory T cells (FOXP3, IKZF2, and IL1RL1), and interferon-responsive cells (OAS2, MX1, and ISG15) (FIG. 3B). Applicant identify 2 primary population of CD8+ T cells: GZMK+ CD8 T cells (GZMK, CCL5, and CXCR4) and effector CD8 T cells (CX3CR1, GZMB, and ZEB2) (FIG. 23). Applicant further 2 populations of T cells that consist of both CD4+ and CD8+ T cells: one population of proliferating T cells (MKI67 and TOP2A) and another population of Th1-Th17 cells. Applicant performed extensive comparison to literature-derived expression signatures from bulk and single-cell RNA sequencing (FIG. 24). Applicants generated gene-expression signatures for numerous T cell clusters across the literature and compared these signatures to granuloma T cells. In most cases, the signature scores were based on the top ˜20 cell-type defining genes. Fundamentally, these comparisons are helpful for understanding T cell state rather than functional programs (FIG. 24). In comparison to signatures from Guo et al. Nature Medicine 2018, Applicants observed similarities between a number of populations including GZMK+ CD8 T cells, Naïve CD4 T cells, NK cells, Effector CD8 T cells and regulatory T cells. Further, Applicants observed enrichment for a signature associated with MAIT cells from Guo et al. in cluster 6, which was also contains TCRs associated with MAIT cells (FIG. 24).

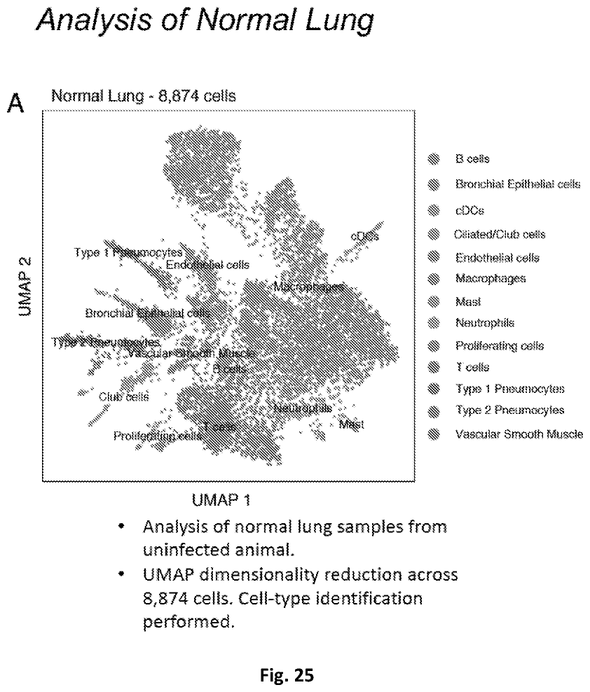

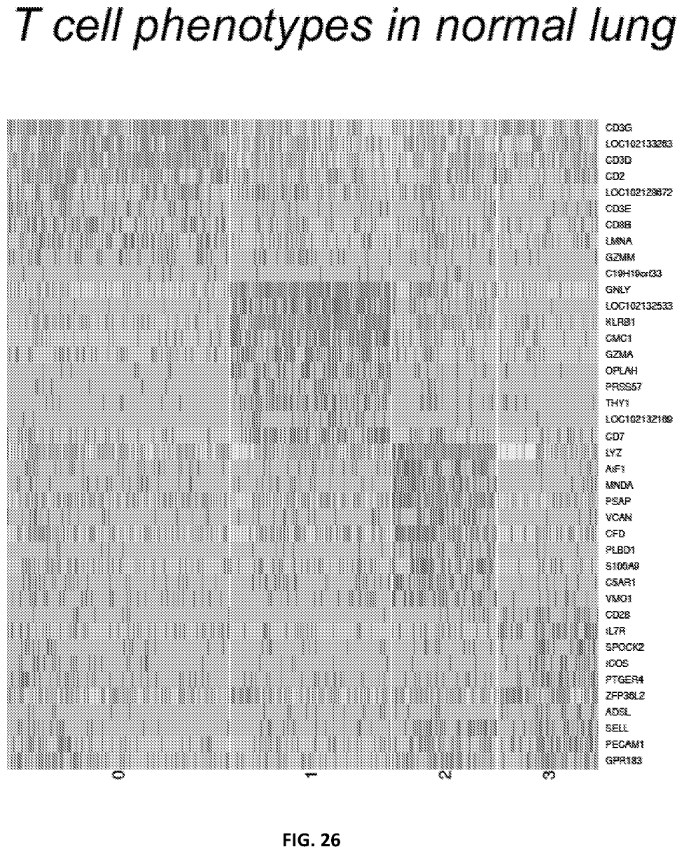

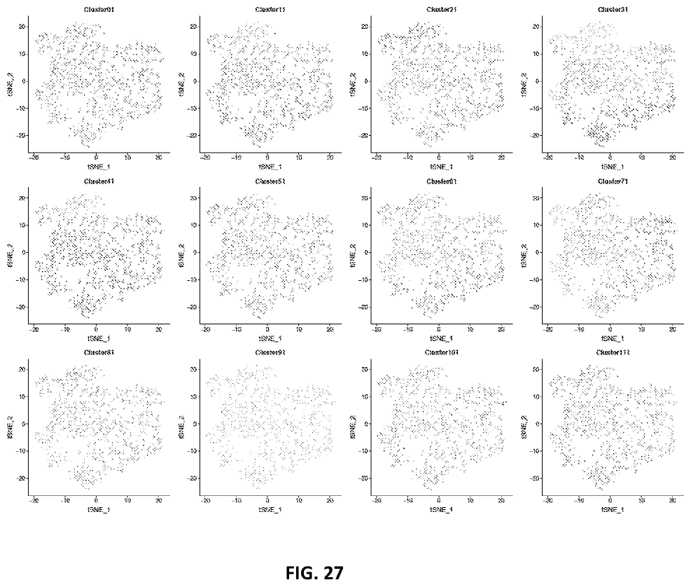

Whether T cell signatures identified in granulomas conserved in normal lung was investigated. Scores from top cluster-defining genes from granulomas (12 clusters in total) were generated. Score T cells from normal lung for expression of granuloma cluster-defining genes (FIG. 25). 4 primary clusters of T cells across the analyzed T cells from normal lung were identified (FIG. 26). In general, there were 2 populations of cytotoxic T cells, and a single CD4+ T cell population (FIG. 26). Comparison of T cells phenotypes in normal T cells (FIG. 27). CD8 T cells in normal lung were most consistent with cytotoxic signatures that define NK cells, GZMK+ T cells, and effector CD8s. Among CD4 T cells, Applicant observed enrichment of signature-defining genes for naïve T cells. In general, Applicant primarily observed CD8 T cells in normal lung. Among CD4 T cells, Applicant observed enrichment of naïve CD4+ T cells based on similarity to granuloma naïve T cell signatures. Notably, we don't observe a population of Th1-Th17 cells present in the normal lung.

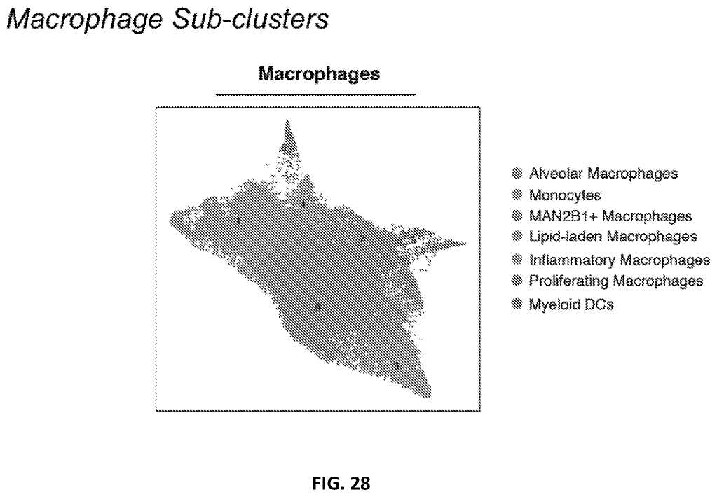

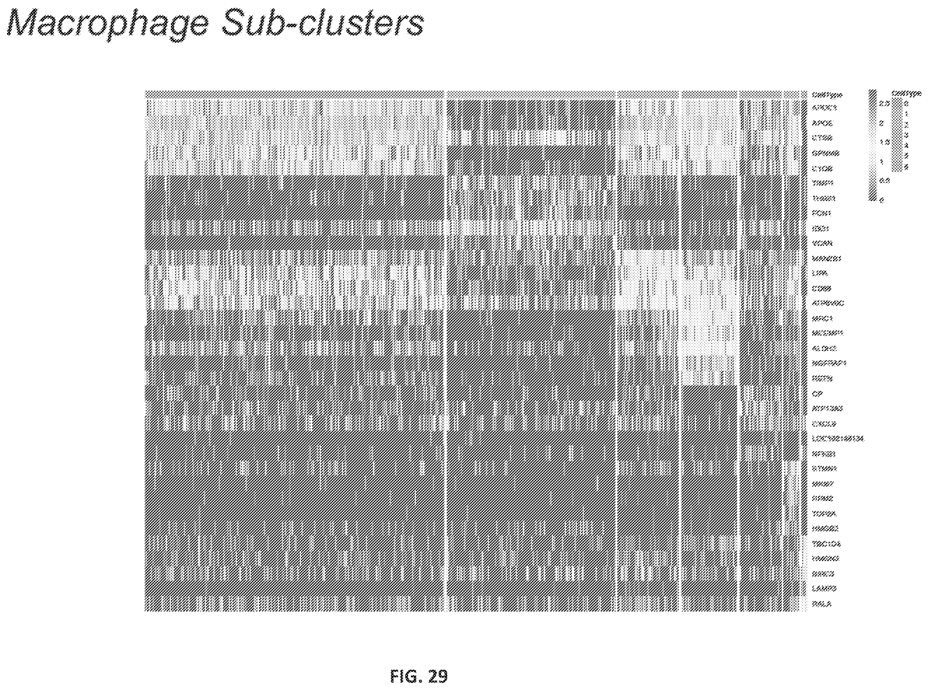

Macrophage population diversity: In macrophages, Applicant performed dimensionality reduction and clustering among 29,993 macrophages and identify 7 phenotypic sub-clusters (FIG. 28). Specifically, Applicant detect a population of alveolar macrophages (APOC1, APOE, C1QB), monocytes (TIMP1 and IDO1), MAN2B1+ macrophages (LIPA and MAN2B1), lipid-laden macrophages (MRC1, FABP5, PPARG), inflammatory macrophages (CP, CXCL9, NFKB1), proliferating macrophages (MKI67 and TOP2A), and myeloid dendritic cells (BIRC3, CCR7, LAMP3) (FIG. 29).

Granuloma Cell-Type Composition is Associated with Granuloma-Level Bacterial Burden

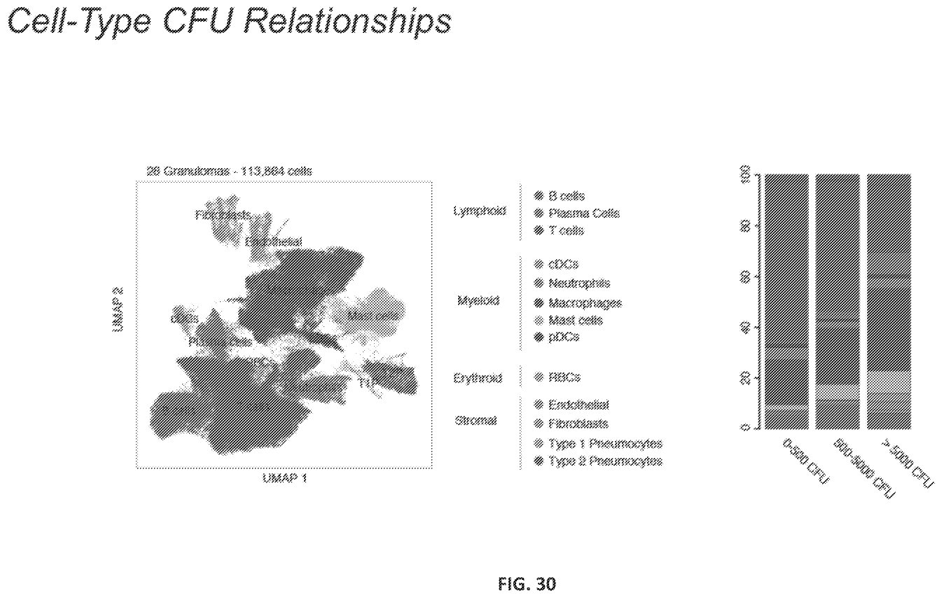

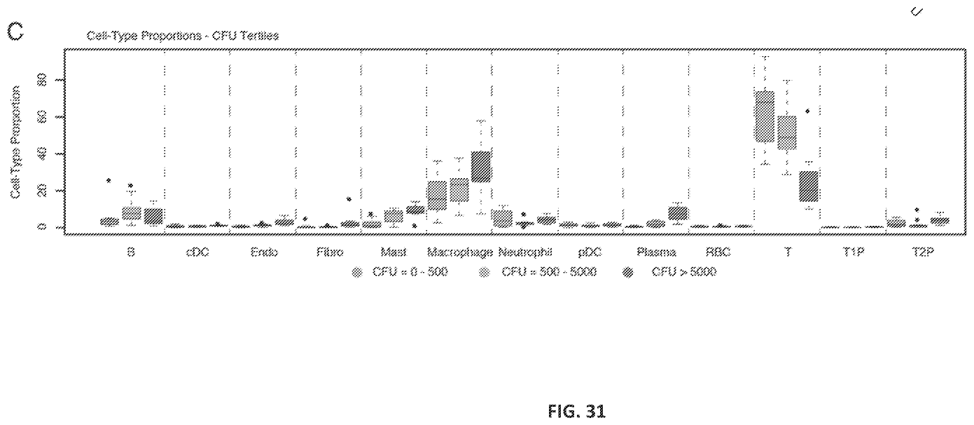

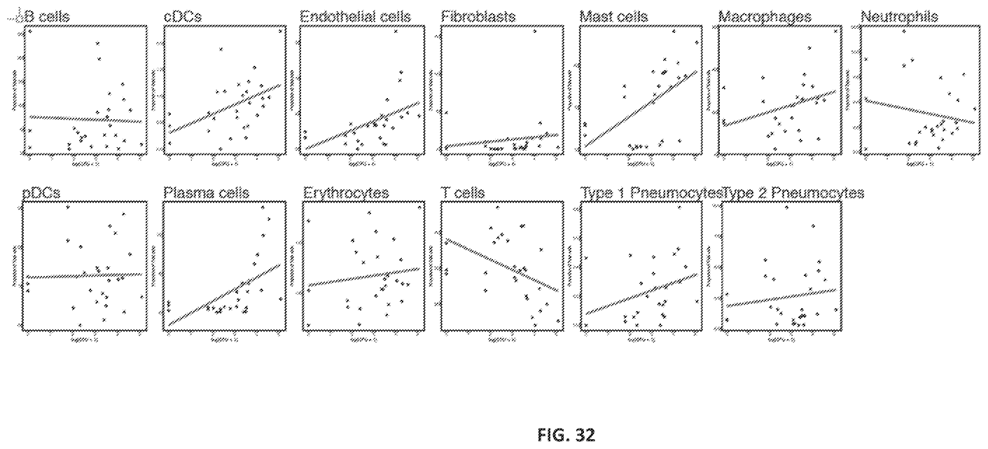

Generic Cell-type Relationships: Applicant observed significant relationships between cell-type between the abundance of multiple cell types and granuloma-level bacterial burden (CFU) and bacterial killing (CFU/CEQ ratio) (FIG. 30; Methods). Using a rank-correlation test, Applicant observed higher proportions of T cells in granulomas with lower end-point bacterial burden (FIGS. 31 and 32). Applicant further observed a non-significant trend of increased bacterial burden with increased proportion of macrophages (FIGS. 31 and 32). Notably, Applicant observed an unexpected relationship between the proportion of mast and plasma cells and increased granuloma-level bacterial burden (FIGS. 31 and 32). Further, Applicant reported significant associations with fibroblasts, endothelial cells, and conventional DCs (FIGS. 31 and 32). For each granuloma, Applicant calculated the proportion of each cell type. Applicant then examined the relationship between % composition of each cell type and granuloma-level CFU across 28 lesions.

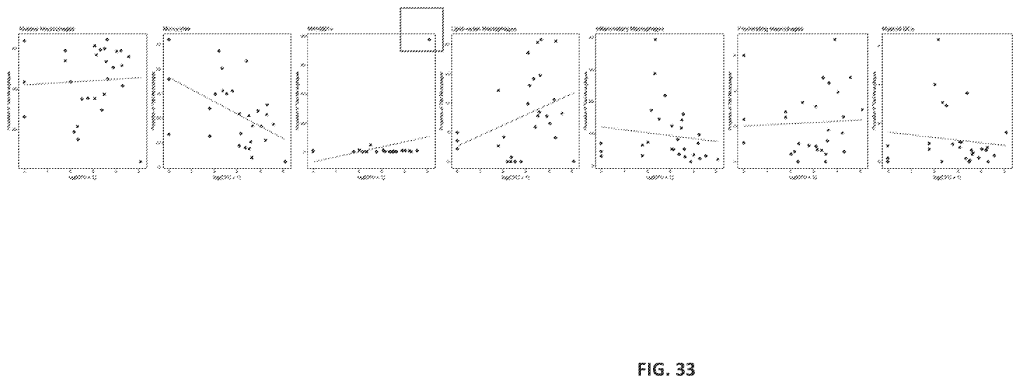

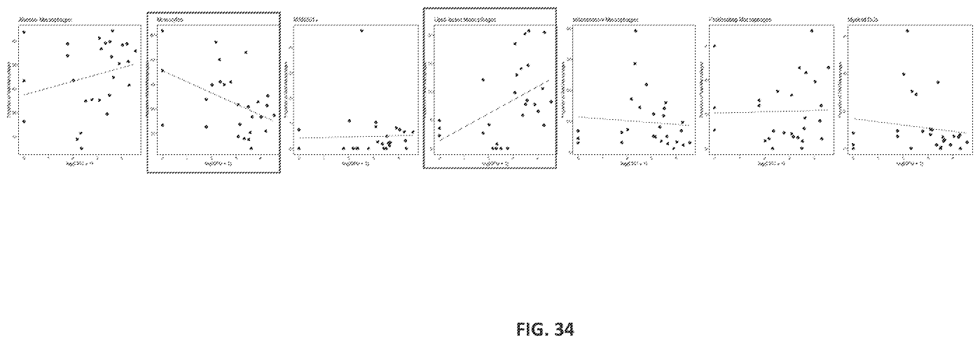

Macrophage CFU Relationships: Applicant observed a suggestive but non-significant relationship between macrophage frequency and granuloma-level CFU (FIG. 33). There was a single lesion that was dominated by MAN2B1 macrophages. Notably, this lesion was the highest burden lesion (CFU=120600), which is a granuloma cluster. If Applicant excluded this outlier lesion (Granuloma 23), significant relationships between Monocytes and lipid-laden macrophages were observed (FIG. 34). Applicant further examined the relationship between composition of macrophage populations and granuloma-level bacterial burden, and did not observe associations between the relative abundance of macrophage sub-populations and granuloma CFU. Applicant observed a granuloma “cluster” with the highest bacterial burden in which the composition was dominated by MAN2B1+ macrophages. Upon exclusion of this outlier lesions, Applicant observed an increased proportion of monocytes in low-burden lesions, while Applicant observed more lipid-laden macrophages in high burden lesions.

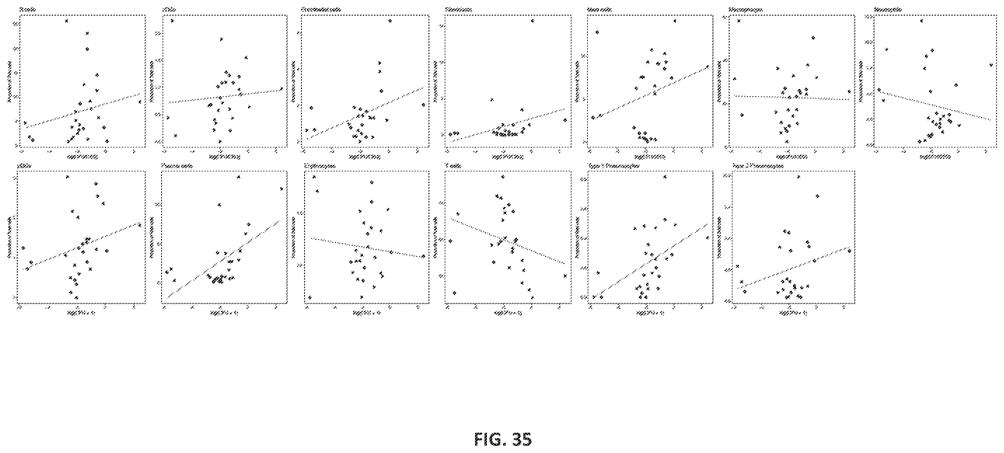

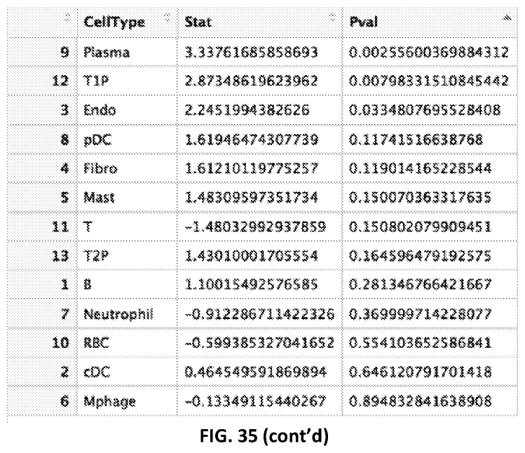

CFU-CEQ relationships: In addition to end-point CFU, Applicant examined the relationship between cell type composition and CFU-CEQ ratio. Applicant observe significant positive relationships between the CFU-CEQ ratio and plasma cells, endothelial cells and Type 1 pneumocytes (FIG. 35). Applicant performed spearman rank correlation tests to examine the relationship between the proportion of each cell-type and log(CFU/CEQ) across 28 granulomas.

T Cell Phenotypic Diversity was Associated with Granuloma-Level Bacterial Control

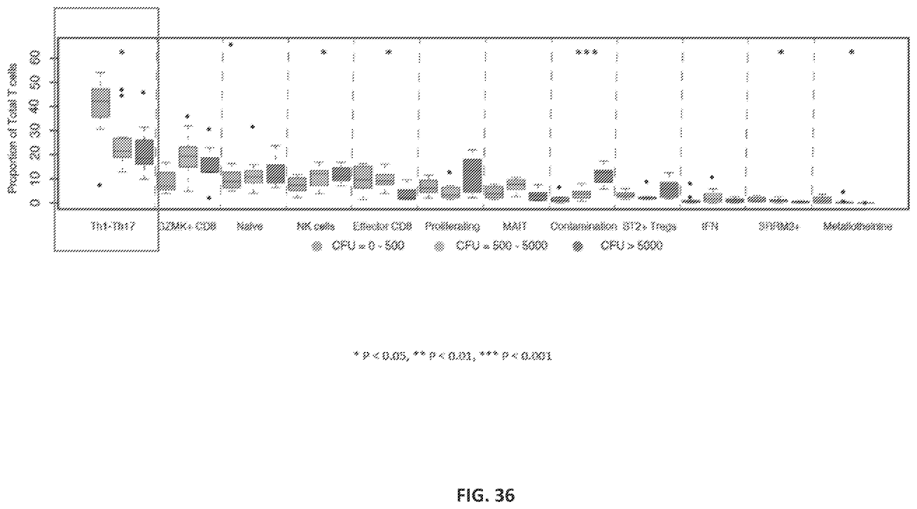

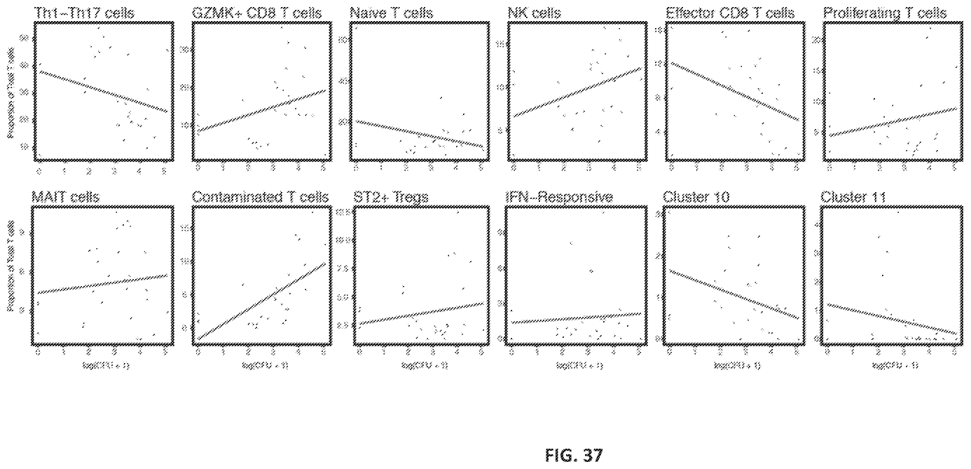

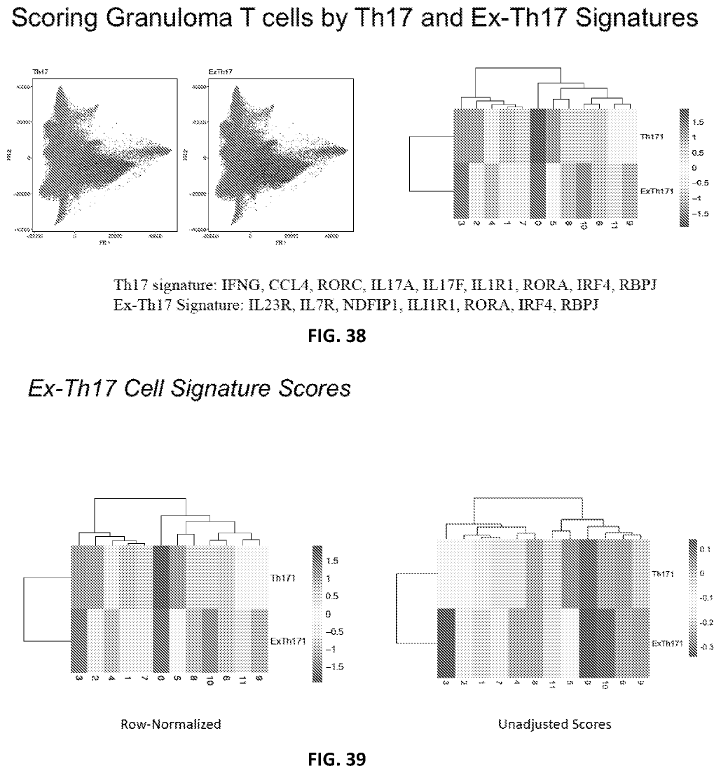

Th1-Th17: Based on the observation that T cells most strongly correlated with protection in aggregate, Applicant sought to understand how the phenotypic composition of T cells across granulomas related to bacterial burden. Th1-Th17 cells had previously been observed to correlate with protective MTB vaccine responses and in clearance of pulmonary infection. Applicant observed a marked expansion of Th1-Th17 cells in granulomas with the lowest bacterial burdens (FIGS. 36 and 37). To understand the functional capacity of Th1-Th17 cells, Applicant examined expression patterns of genes associated with Th1 and Th17 effector function. Among Th1-Th17 cells, Applicant observed enrichment of genes associated with pathogenic Th17 function (BHLHE40, SATB1 and RBPJ) (FIGS. 38 and 39).

Applicant performed differential expression between high and low burden lesions. Applicant further examined correlations between CFU and gene expression across all Th1-Th17 cells.

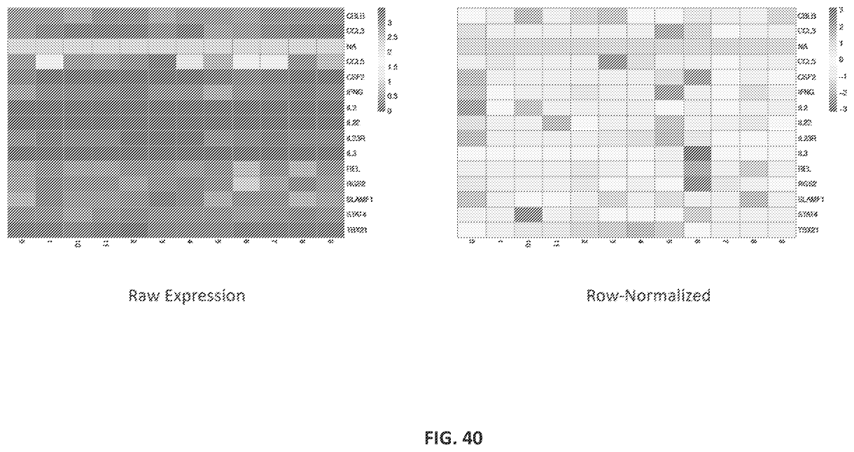

Distribution of Genes that Distinguished Th17 and Th1-Th17 Cells was Examined (FIG. 40)

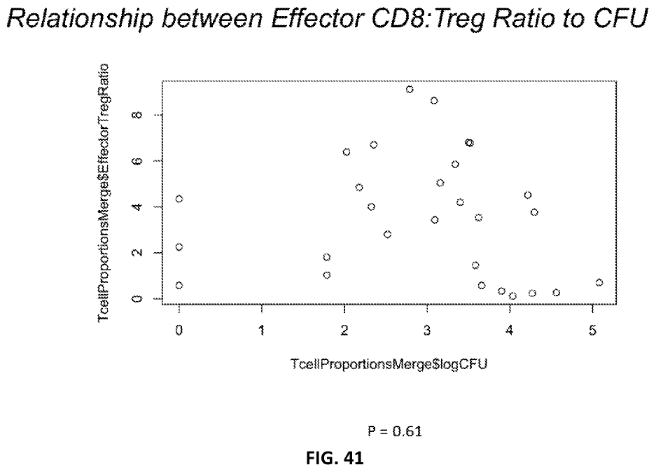

Effector CD8: Applicant also observed significant increases in the frequency of effector CD8 T cells in low-burden lesions. Effector CD8 T cells were marked by elevated expression of transcription factors (KLF2), surface receptors (TGFBR3, CX3CR1) and cytotoxic effector molecules (GZMB). The balance between regulatory and effector function might influence granuloma-level bacterial burden. Applicant did not observe a relationship between the ratio of Effector CD8 T cells to Tregs and granuloma-level CFU (FIG. 41).

T cell Phenotypes—Animal 4017: Applicant examined the distribution of T cell phenotypes within a single animal with the broadest distribution of bacterial burden. In this animal, Applicant observed expansion of proliferating and ST2+ regulatory T cells among high burden lesions in addition to the association of Th1-Th17 and effector CD8 T cells. Among ST2+ Tregs, Applicant observed expression of canonical regulatory T cell genes (FOXP3, TIGIT, GITR) as well as expression of Th2-associated genes (GATA3). While regulatory T cells were associated with reduced bacterial clearance in mice, a positive relationship between regulatory T cells and elevated bacterial burden was only observed in Animal 4017.

T cell Functional Analysis: Applicant sought to understand differences in functional potential of T cell subsets between high and low burden granulomas. Applicant examined differences in expression of cytotoxic effector molecules and canonical markers of T cell exhaustion between high and low burden lesions. Within T cell subsets, Applicant did not observe differential expression of PDCD1, HAVCR2, or TOX between high and low burden lesions. Applicant further examined the distribution of cytotoxic gene expression within GZMK+ CD8 T cells, effector CD8 T cells and NK cells between high and low burden lesions. In these comparisons, Applicant failed to observe significant differences in expression of cytotoxic effector molecules (e.g. GNLY, GZMB, and GZMK). Applicant further performed differential expression analysis within each T cell clusters to identify genes up-regulated in either TB-restrictive or permissive lesions. In these analyses, Applicant did not observe strong functional differences within T cell subsets obtained from high or low burden lesions. These findings suggested that relative composition of T cell phenotypes was a primary contributor to bacterial control rather than gain/loss of function within T cell subsets.

Approach to Understanding T Cell States (Identity)

Definition: Expression program that is central to cellular identity that varies between T cell subsets, but will be invariant within T cell subsets. (e.g. Effector CD8 T cells by virtue of being Effector CD8 T cells express high levels of cytotoxic effector molecules). Between Cluster Differential Expression (Relative): To understand how functional programs vary across T cell subsets. For example, NK cells and effector CD8 T cells express higher levels of cytotoxic effector molecules relative other T cell clusters. In this case, this reflects both an identity (i.e. cytotoxic lymphoid cells) as well as a functional program (cytotoxic function). In this case, applicant inferred the function of restrictive/permissive T cell subsets based on their relative expression of functional T cell programs/effector molecules. Applicant examines the expression of cytokines and effector molecules.

Approach to Understanding T Cell Function (Differential Expression)

Definition: Expression program that is secondary to cellular identity that can vary within a given T cell population. (e.g. T cell Exhaustion, Effector function, cytokine production, etc.). Within Cluster Differential Expression: To understand how functional programs vary between cells derived from high and low burden lesions.

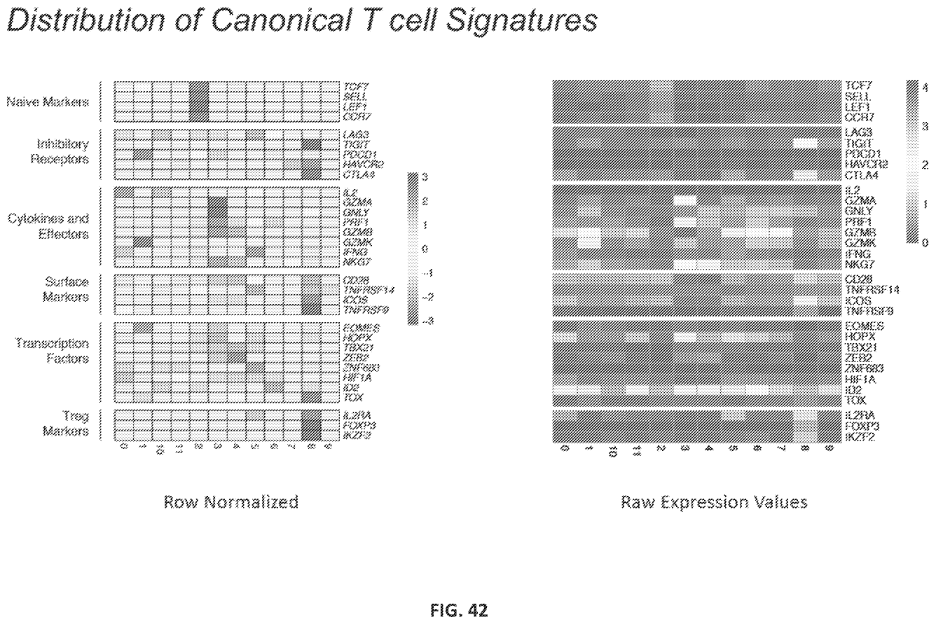

Distribution of canonical T cell genes across cell populations was determined (FIG. 42).

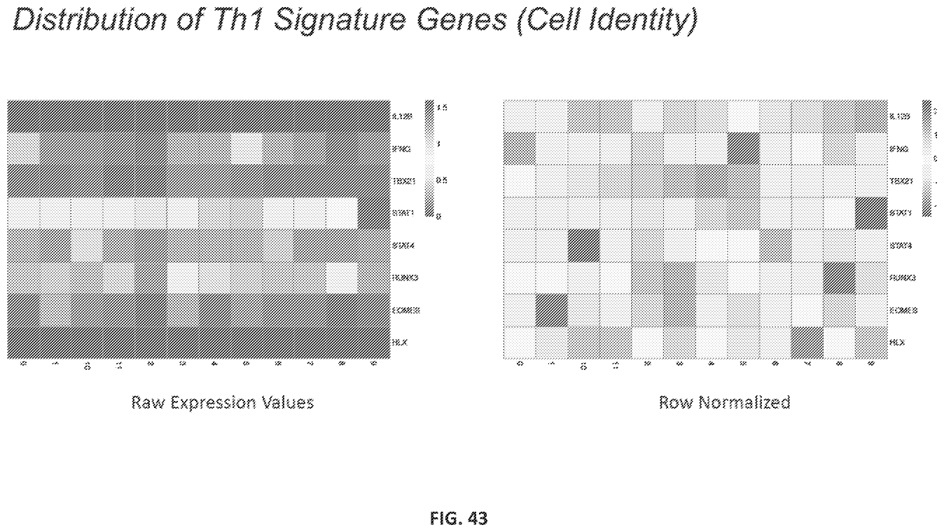

Applicant failed to observe clear signatures of purely Th1 polarized T cells. Instead, Applicant observed highest expression of STAT1 among IFN-responsive T cells (T cell cluster 9) (FIG. 43).

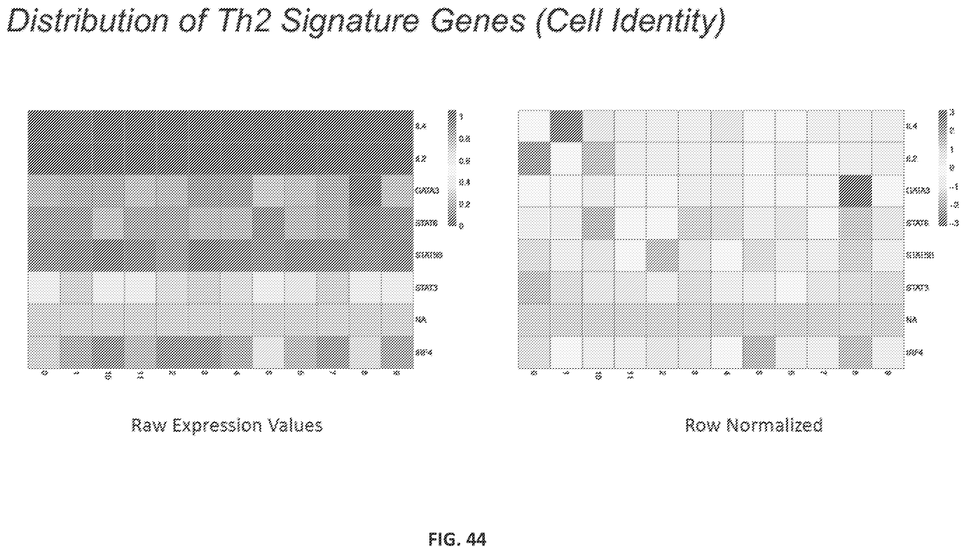

Applicant observed the highest expression of GATA3, the Th2 lineage defining cytokine, among a population of ST2+ Tregs. However, Applicant failed to detect appreciable levels of Type 2 cytokine production from any T cell cluster (FIG. 44).

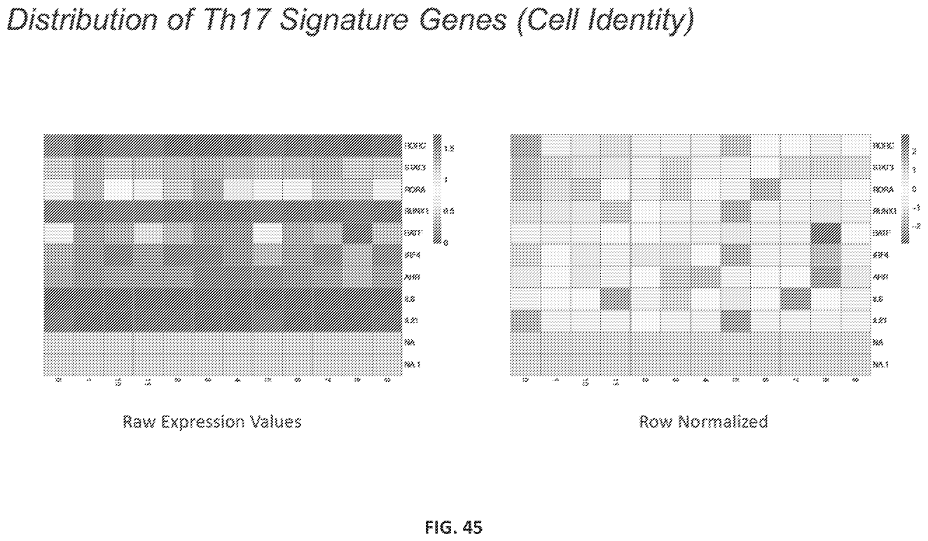

Applicant observed the highest expression of multiple type 17 genes primarily cells from Clusters 0 (Th1-Th17) and cluster 5 (proliferating T cells) (FIG. 45).

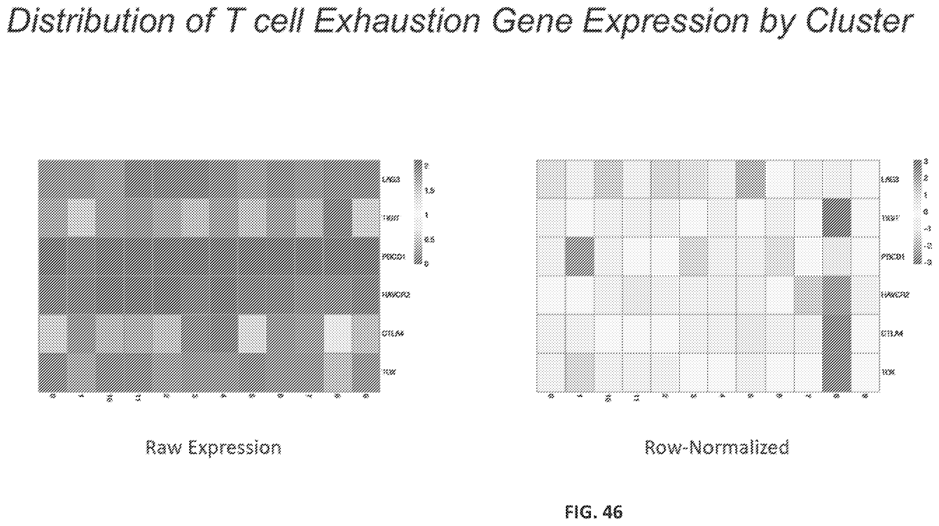

Examination of the distribution of canonical exhaustion markers across T cell clusters. Applicant examined the distribution of PDCD1, HAVCR2, LAG3, TOX, CTLA4, and TIGIT (FIG. 46). Applicant observed highest expression of inhibitory receptor expression in ST2+ regulatory T cells. While Applicant observed the highest relative expression of PD1 in GZMK+ CD8 T cells, the absolute expression was low. While Applicant observed the highest relative expression of PDCD1 in GZMK+ CD8 T cells, the overall expression of PDCD1 was low relative to other inhibitory surface receptors. For example, Applicant observed the highest levels of expression of TIGIT and CTLA4 on ST2+ Tregs rather than CD8 T cells.

Applicant identified exhausted cells on the basis of co-expression of TIM3, PD1 and TOX. [0378] Applicant examined the distribution of T cell exhaustion between high and low-burden lesions with particular focus on GZMK+ CD8 T cells and Effector CD8 T cells. This approach revealed very few cells to be exhausted, as co-expression of 3 lowly expressed markers quickly diminishes the number of cells. (Only 2/46640 T cells co-express all 3 markers). If co-expression of any 2 of 3 markers is used as to determined exhaustion 133/46640 T cells were “exhausted”.

Benchmarking Against Differential Expression Results—Applicant further examined the distribution and differences in expression of canonical exhaustion markers in both GZMK+ CD8 T cells and effector CD8 T cells. In both cases, Applicant failed to observe significant differences in the expression of canonical exhaustion genes (PDCD1, HAVCR2, TOX, LAG3).

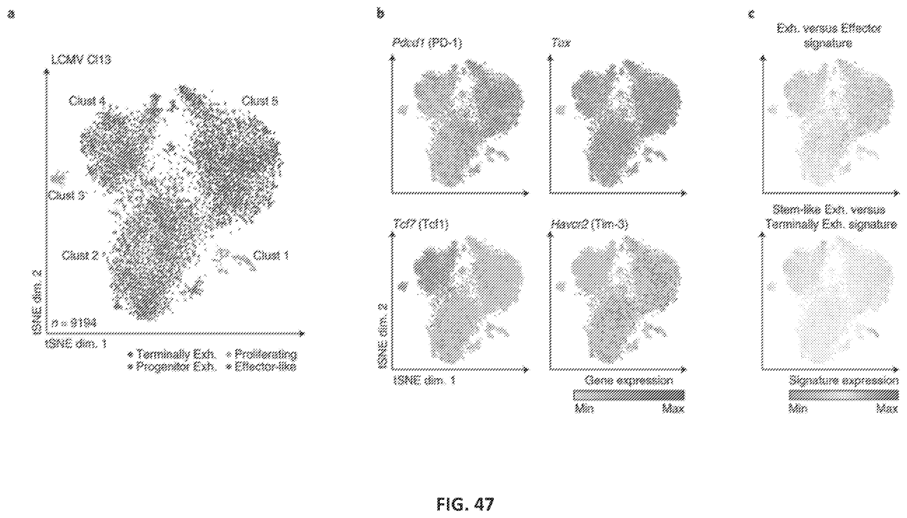

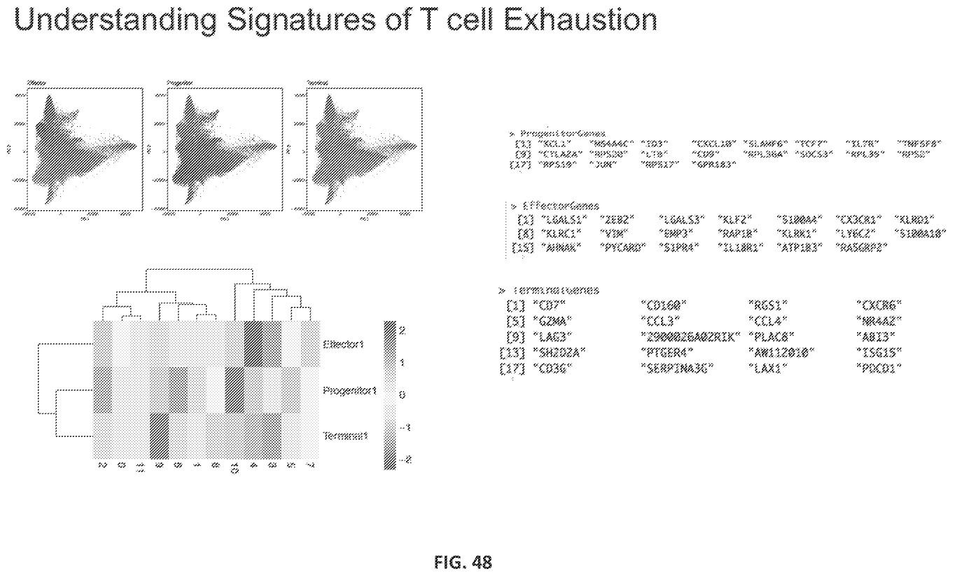

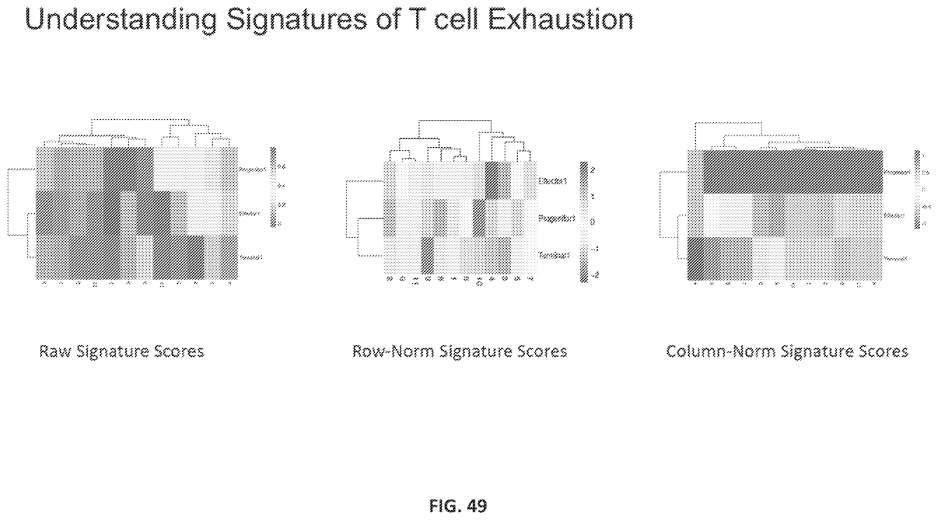

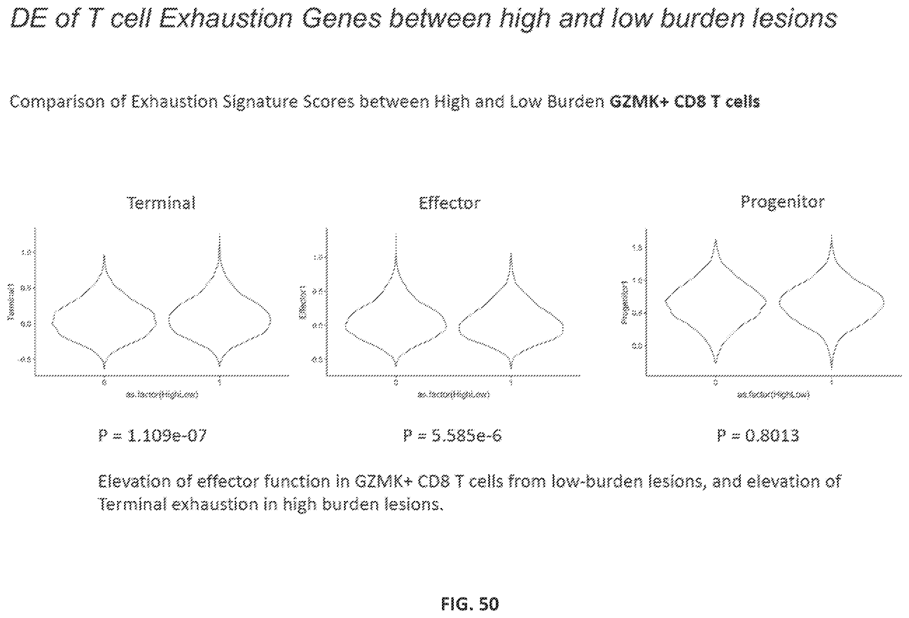

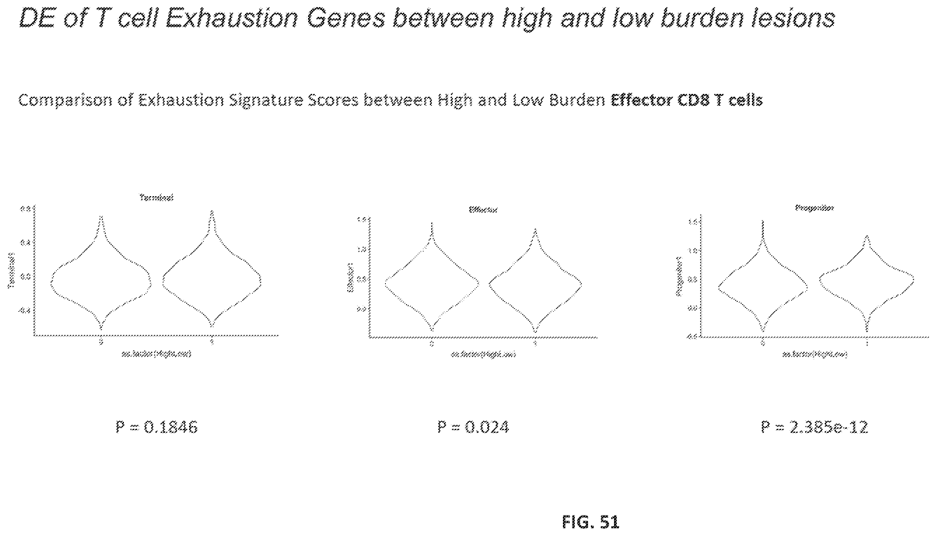

Distribution of exhaustion signatures from Miller et al. Nature Immunology 2019 was examined (FIG. 47). T cell exhaustion was examined (FIGS. 48-49). Comparison of exhaustion signature scores between high and low burden GZMK+ CD8 T cells was performed (FIG. 50). Comparison of exhaustion signature scores between high and low burden effector CD8 T cells (FIG. 51).

Applicant observed the highest overall expression of many exhaustion markers among regulatory T cells. “Gating-based” identification of Exhausted CD8 T cells in either GZMK+ or Effector CD8 populations using co-expression of canonical exhaustion markers TIM3 (HAVCR2), PD-1 (PDCD1), and TOX resulted in extremely low numbers of exhausted T cells. Relaxing the stringency of co-expression requirements results in higher overall #s of “exhausted” T cells. Using these numbers, the proportion of exhausted and non-exhausted T cells was similar between GZMK+ CD8 T cells derived from high and low burden lesions. Literature-based signatures of exhaustion (Miller et al. Nat. Immunology 2019) were examined specifically within GZMK+ CD8 T cells and Effector CD8 T cells. Within the GZMK+ CD8 T cells, Applicant observed elevated expression of terminal exhaustion signatures in high-burden T cells. However, while these differences were statistically different, the effect size or lack thereof likely precluded biological meaning. Similarly, Applicant observed a slight (but significant) increase in effector signature score in GZMK+ CD8 T cells.

Applicant initially examined the distribution of cytotoxic effector molecules across T cell clusters.

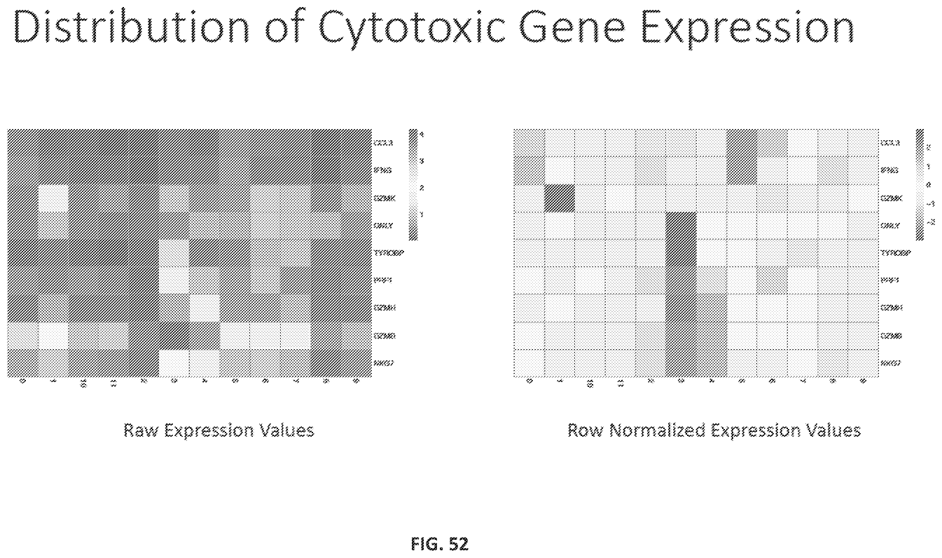

Applicant observed the highest degree of cytotoxic gene expression among NK cells. Notably, Applicant observed the highest levels of granulysin, perforin and granzyme B expression in NK cells. However, effector CD8 T cells express reduced/absent levels of granulysin perforin. Finally, Applicant observed the highest levels of GZMK expression in the GZMK+ CD8 T cell cluster (FIG. 52).

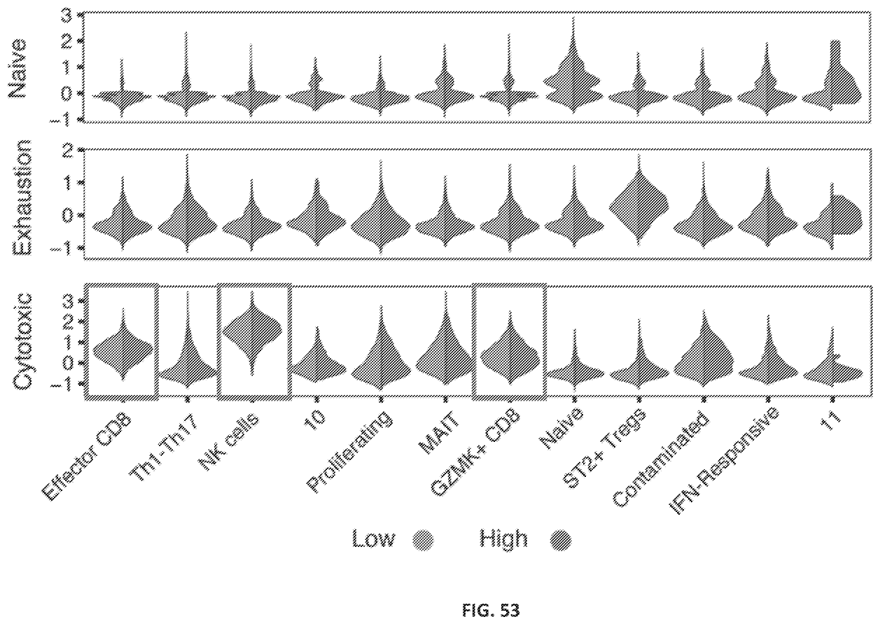

To examine potential differences in cytotoxic gene expression between high and low burden lesions. Specifically, Applicant generated a cytotoxic expression score using the genes (FIG. 53). Among cell types with the highest levels of cytotoxic gene expression (NK cells, Effector CD8 T cells, and GZMK+ CD8 T cells, Applicant observed similar levels of cytotoxic gene expression patterns between high and low burden lesions. The absence of difference in cytotoxic expression suggests that these genes represented a core set of genes that reflected the “identity” these cells rather than a unique aspect of function in the diverse granuloma environments.

Functional enrichment of differentially expressed genes—Applicant identified genes that were differentially expressed within each T cell cluster between high and low burden lesions. To understand potential enrichment of functional pathways that were overrepresented (distinct from the underlying T cell subtype identity), Applicant performed gene-set enrichment analysis among differentially expressed genes.

Differential expression analysis did not reveal significant insight into functional differences between high and low-burden lesions. In most cases, differences in gene expression between high low burden lesions were dominated by ribosomal, mitochondrial and soup-defining genes. In particular, the analysis had focused on T cell clusters associated with bacterial burden (Th1-Th17, NK Cells, Effector CD8 T cells).

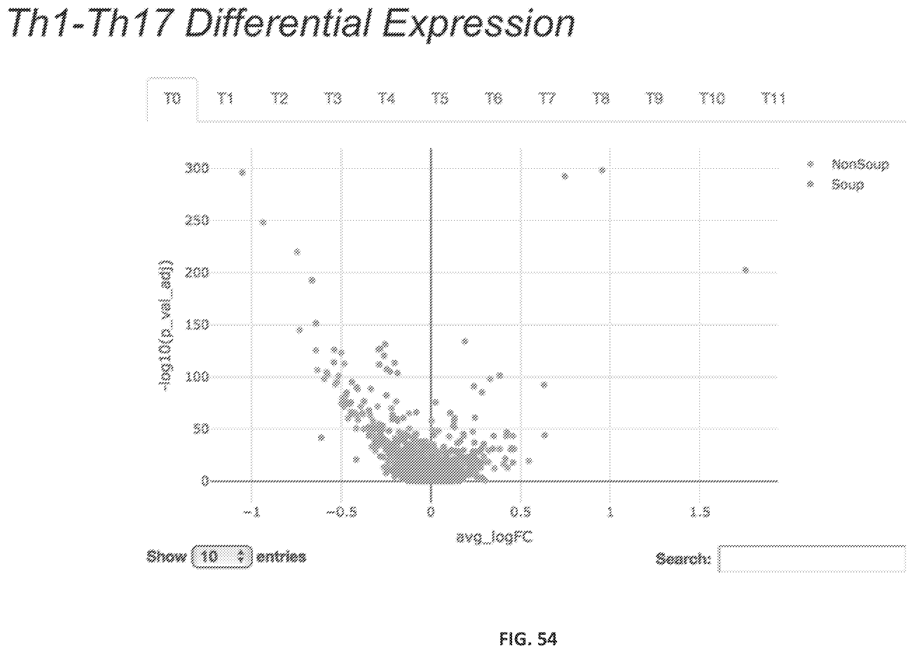

Th1-Th17: Soup-defining genes were significantly enriched in high-low differential expression analyses (FIG. 54). Genes over-expressed among high-burden Th1-Th17 cells included: IL2RG, IFNG, IFI27, LAG3, TIGIT, CD8A, NKG7, CCL20, CCL3, CCL5. These differences in gene expression were driven by the presence of a sub-cluster of primarily CD8+ T cells within the Th1-Th17 cluster.

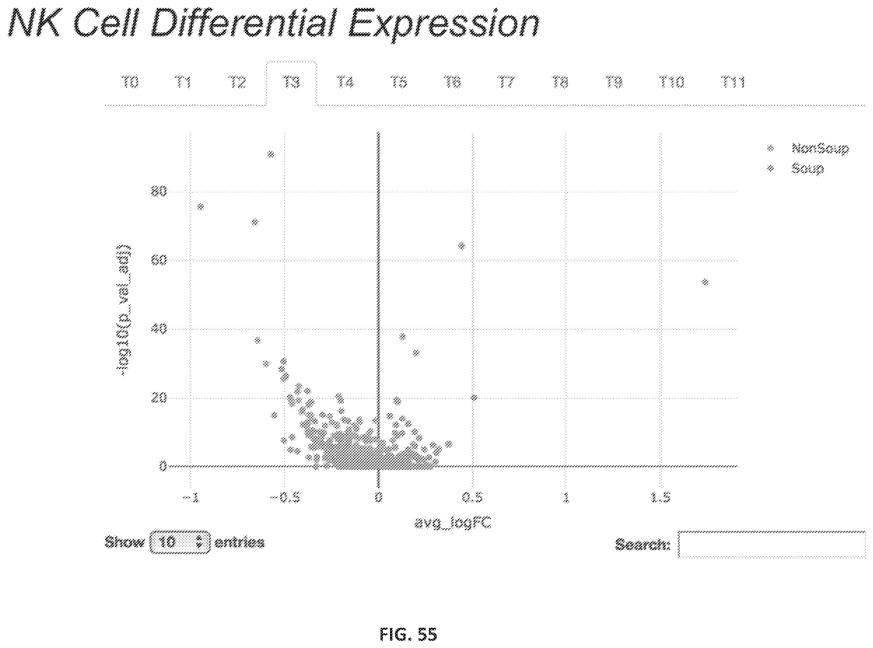

NK Cells: Soup-defining genes are significantly enriched in high-low differential expression analyses (FIG. 55).

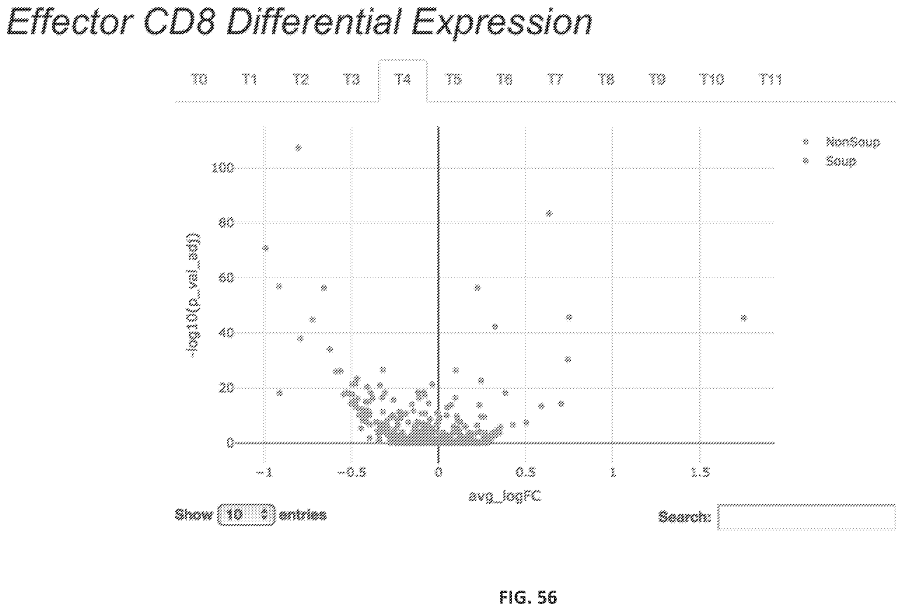

Effector CD8 T cells: Soup-defining genes are significantly enriched in high-low differential expression analyses (FIG. 56).

Expression of Cytokines and Effector Molecules in T cells subsets—Applicant calculated the proportion of each T cell sub-type relative the total number of T cells in each lesion. Then Applicant examined the relationship between % composition of each T cell sub-cluster (i.e. proportion of total T cells with each granuloma) and granuloma-level bacterial burden across 28 granulomas. Applicant performed differential expression analysis to explore differences in expression of key cytokines across clusters between high and low burden lesions.

Applicant sought to understand differences in functional potential of T cell subsets between high and low burden granulomas. Applicant examined differences in expression of cytotoxic effector molecules and canonical markers of T cell exhaustion between high and low burden lesions. Within T cell subsets, we did not observe differential expression of PDCD1, HAVCR2, or TOX between high and low burden lesions. Applicant further examined the distribution of cytotoxic gene expression within GZMK+ CD8 T cells, effector CD8 T cells and NK cells between high and low burden lesions. In these comparisons, Applicant failed to observe significant differences in expression of cytotoxic effector molecules (e.g. GNLY, GZMB, and GZMK). We further performed differential expression analysis within each T cell clusters to identify genes up-regulated in either TB-restrictive or permissive lesion. In these analyses, Applicant did not observe strong functional differences within T cell subsets obtained from high or low burden lesions. These findings suggest that relative composition of T cell phenotypes is a primary contributor to bacterial control rather than gain/loss of function within T cell subsets.

Clonotype-Phenotype Relationships Influence Bacterial Control in MTB Granulomas

TCR Recovery: Differences in antigen specificity might contribute to differences in bacterial burden between high and low burden lesions. To understand the extent of T cell clonality and its relationship to MTB-restrictive T cell phenotypes, Applicant performed enrichment of alpha and beta TCR sequences from whole-transcriptome amplification libraries and recovered CDR3 sequences through targeted sequencing (Methods).

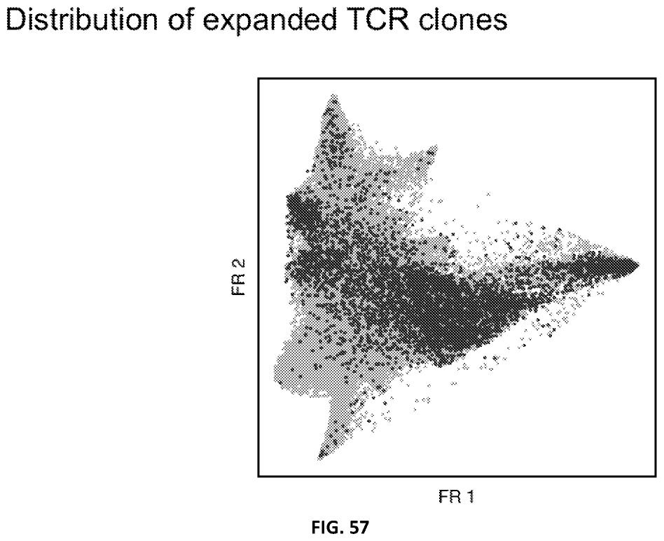

Extent of Clonal Expansion: Applicant compared the extent of clonal expansion between high and low burden lesions. Applicant identified expanded TCR clones as those clones occurring more than once within an animal. In high burden lesions, Applicant observed [higher/lower/similar] levels of clonal expansion compared to low-burden lesions (FIG. 57).

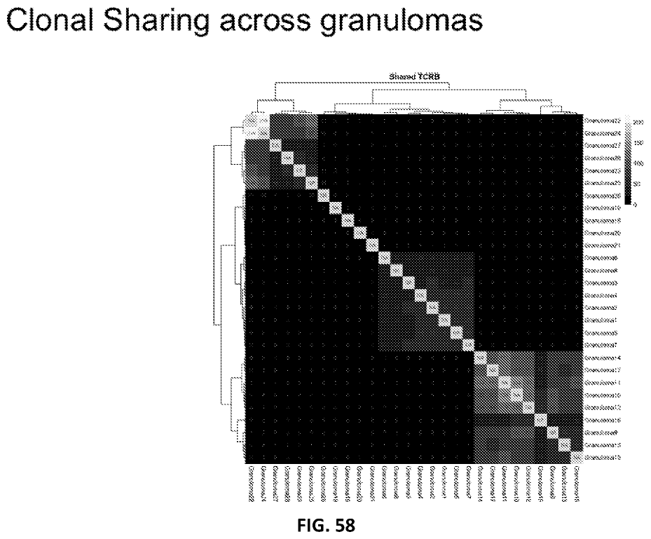

Clonal Sharing between Granulomas: Applicant further examined the extent of clonal sharing between high and low Applicant examined the extent of clonal expansion between high and low burden lesions. Applicant identified expanded TCR clones as those clones occurring more than once within an animal. In burden lesions within animals, Applicant observed a high degree clonal sharing between high and low burden granulomas (FIG. 58). In cases in which Applicant observed clonal sharing between high and low burden lesions, Applicant examined both the extent of clone expansion and contraction. For example, in animal 4017, Applicant calculated a metric of clonal diversity and performed comparison between high and low burden lesions.

Convergent clonotype selection: Applicant sought to understand convergent clonotype selection, and Applicant examined similar TCR sequences (convergent clonotype selection) to understand how shared antigens might drive the expansion of multiple clones. In this analysis

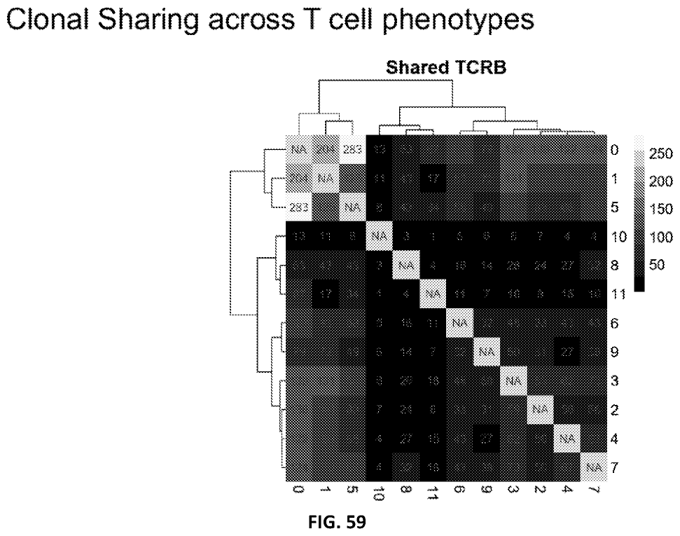

TCR Phenotype Relationship: Applicant next sought to understand the relationship between TCR clonality and T cell phenotype and found the highest degree of clonal expansion among GZMK+ CD8 T cells, effector CD8 T cells, Th1-Th17 cells and proliferating T cells (FIG. 59). Applicant observed the highest extent of clonal expansion within Th1-17 cells, Effectors CD8 T cells, GZMK+ CD8 T cells, and proliferating T cells. When Applicant examined the phenotypic landscape of individual TCR clones, Applicant observed that individual clones are largely limited to a single T cell phenotype.

To determine the distribution of phenotypes within expanded clones, Barplot was made to show the percent phenotypic composition of each clone (CDR3 sequence) irrespective of granuloma of origin. The barplot was ranked by degree of expansion.

To determine whether this distribution vary based on bacterial burden, barplot showing the percent phenotypic composition of each clone (CDR3 sequence) between high and low burden lesions was made and ranked by degree of expansion.

Identification of DURTS: Applicant used TCR sequences to identify rare populations on donor-unrestricted T cells (DuRTs), which included MAIT cells, GEMs, iNKTs. Applicant observed the highest frequency of T cells bearing MAIT-associated TCRs (TRAV1-2/TRAJ1-33) in T cell cluster 6. Applicant identified MAIT cells (and iNKT cells) based on 2 primary strains of evidence: 1. Enrichment of TCR VJ regions associated with MAIT cells. 2. Similarity to literature-based expression signatures of MAIT cells (Guo et al., Nature Medicine 2018).

Gamma-Delta T cells: Applicant further examined the presence and distribution of gamma delta T cells across granulomas. Applicant identified gamma-delta T cells based on expression of either gamma or delta constant genes.

In-Situ Validation of Cell-Type Composition

To validate the presence and relative abundance of cell-types in MTB granulomas, Applicant performed IHC staining on a matched set of non-human primate granulomas with known bacterial burden (Methods).

Relationship Between Timing of Granuloma Formation and Granuloma Composition

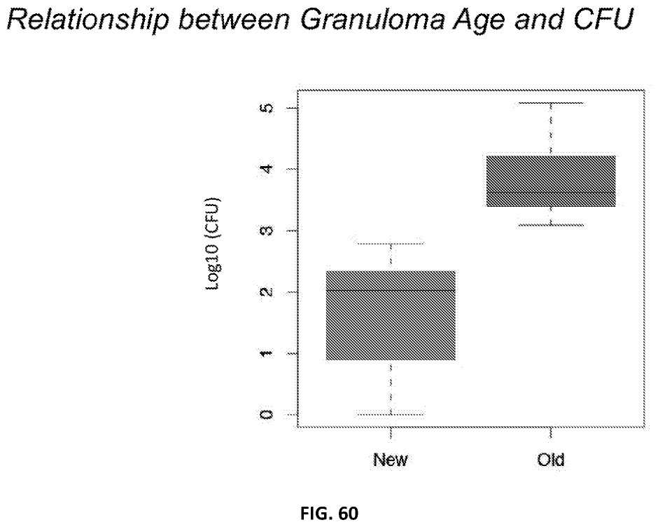

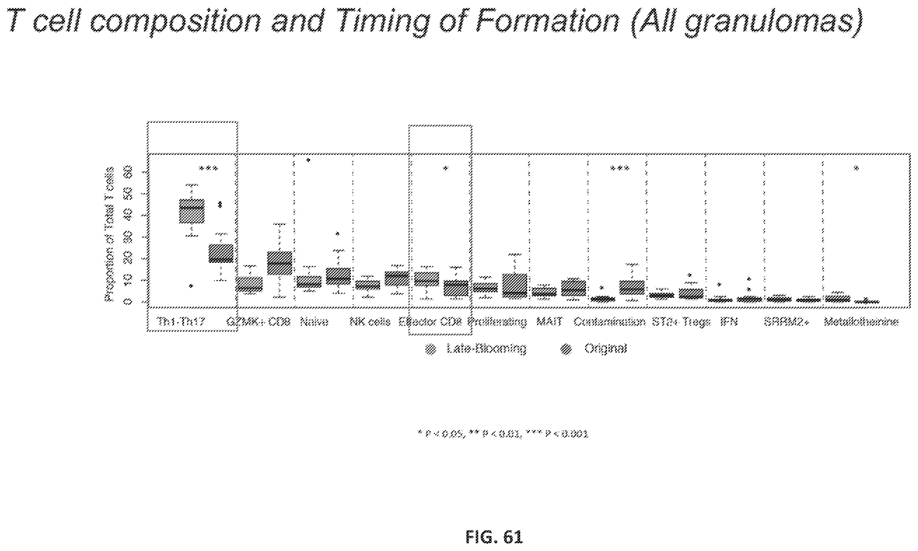

CFU-Age Association/Spreading Lesions: For a subset of lesions, Applicant observed granulomas only at 10-week scans immediately prior to necropsy (FIG. 60). Applicant examined the distribution of overlapping bacterial barcodes within the late-blooming lesions and observed enrichment of ‘spreading’ lesions in this group. Among the ‘late-blooming’ lesions Applicant observed significantly lower bacterial burden. Comparison of cell-type frequencies between original and late-blooming granulomas.

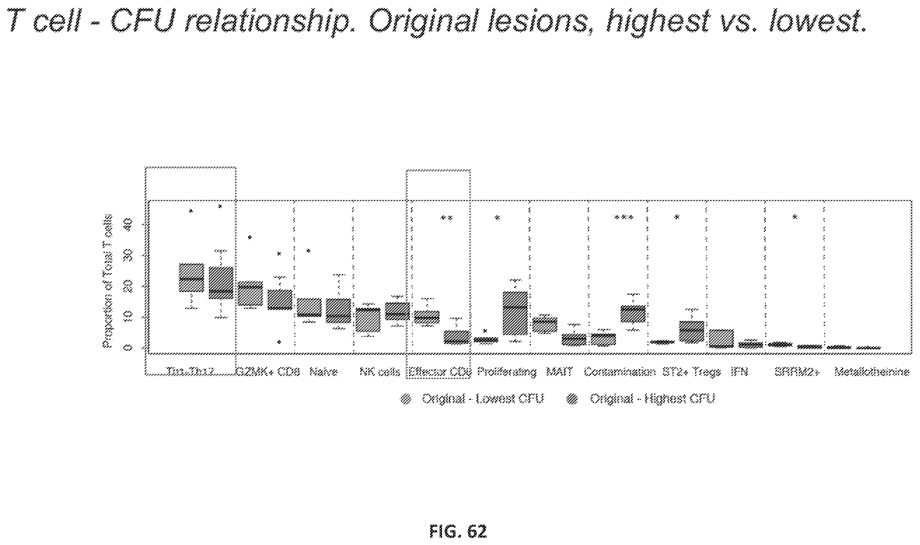

Age-CFU Associations viz. T cell populations: To understand how temporal dynamics of granuloma formation influence associations with bacterial burden, Applicant examined the distribution of T cell phenotypic states between lesions that were observed at all PET-CT timepoints with the highest and lowest bacterial burdens (FIGS. 61 and 62 and Methods). Among lesions observed only at 10-weeks post-infection, Applicant observed the lowest bacterial burdens, despite similar cumulative bacterial burdens. In comparison of early-forming and late-blooming lesions, Applicant observed similar associations with Th1-Th17 cells and effector CD8 T cells. However, among early-forming lesions, Applicant observed burden association with effector CD8 T cells but fail to observe a significant relationship for Th1-Th17 cells. These data suggested that early killing by CD8 T cells and late-onset adaptive Th1-Th17 differentiation represented distinct paths to bacterial control.

Ecological Composition Between High and Low-Burden Granulomas

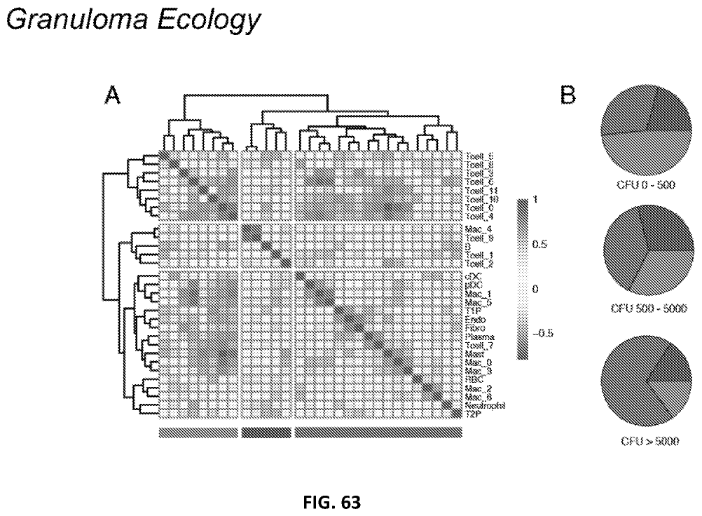

Correlation of cell populations across granulomas: Applicant examined coordinated shifts in cell type composition across granulomas by calculating pairwise Pearson correlation coefficients using cell-type proportions (Methods, FIG. 63). Across the complete set of 28 granulomas, Applicant observed 3 primary groups of cell-types that correspond closely to shifts in bacterial burden across lesions. Specifically, Applicant observed coordinated expansion of T cell sub-populations (Group 1) in low-burden lesions, while in high burden lesions Applicant observe broad increases in myeloid and stromal population (Group 3).

Cell-Cell Interactions Correlate with Granuloma-Level Bacterial Burden

Cell-Cell Interaction Networks: To broadly profile differences in cell-cell interactions between high and low burden lesions, Applicant constructed edge weights for receptor-ligand pairs. For each potential interacting cell-type pair Applicant adjusted receptor-ligand edge weights to account for differences in the abundance of the sender cell type, relative receptor expression, and the percent of receptor positive cells (Methods). Applicant performed comparisons within cell-cell interaction networks to identify receptor-ligand edge weights over-represented in high or low burden lesions.

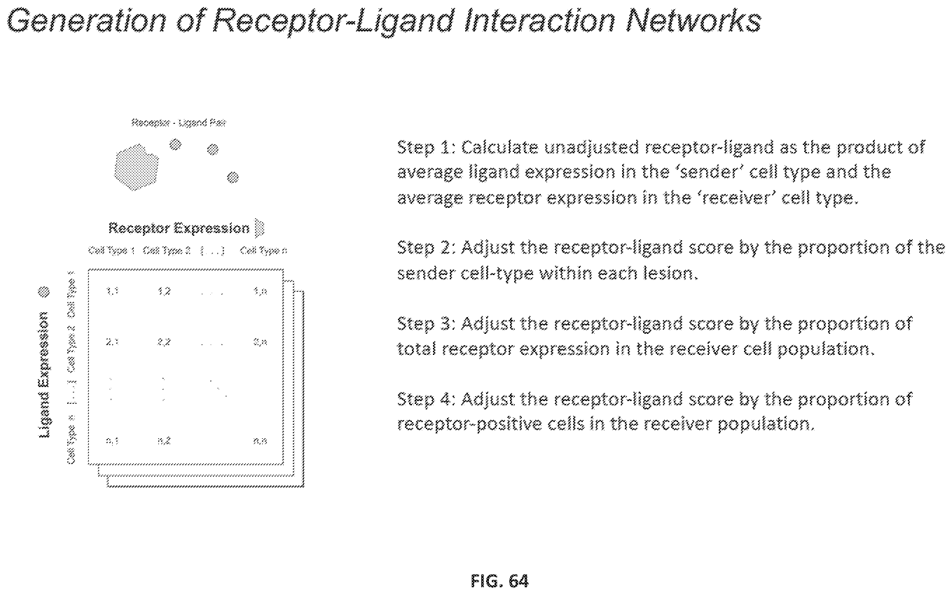

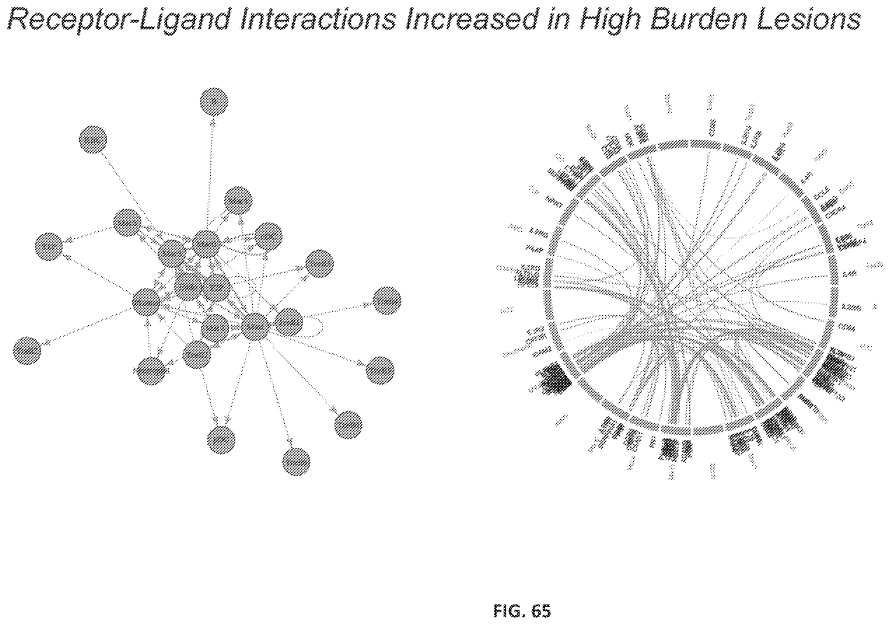

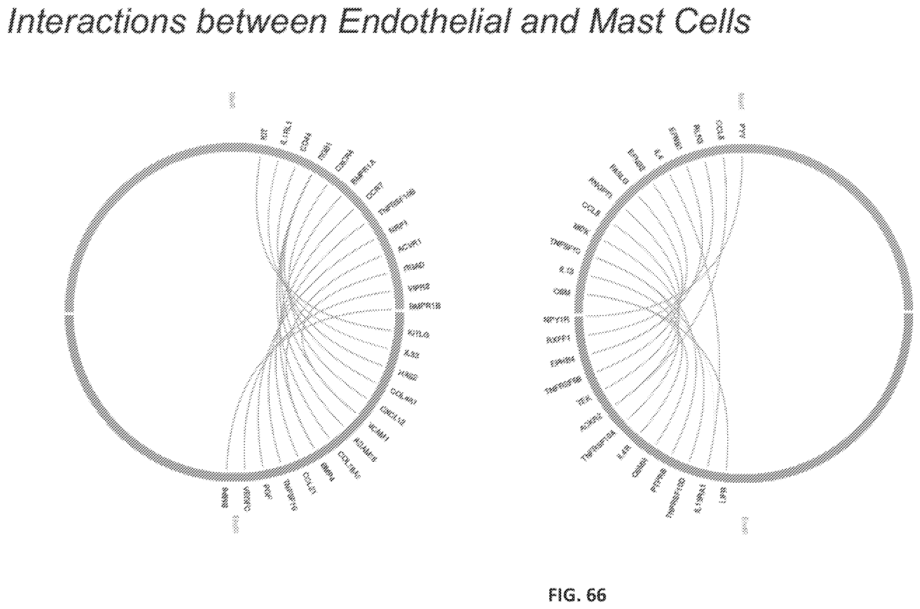

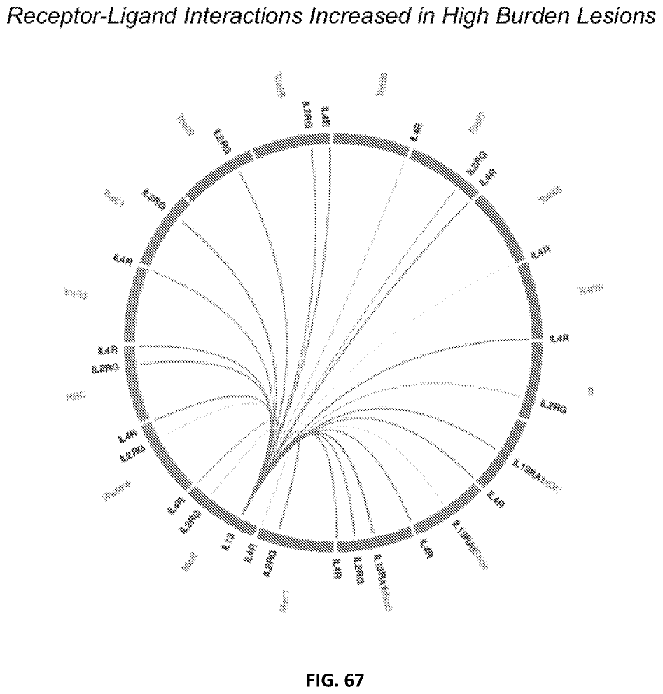

Receptor-Ligand Interactions in High Burden Lesions: Initially, Applicant examined interacting cell partners over-represented in high burden lesions by summarizing receptor-ligand edge weights (FIG. 64, Methods). Applicant generated a network map of cell-cell interactions generated from receptor-ligand edge weights that are significantly different between high and low burden lesions (over-represented in high burden). In high burden lesions, Applicant broadly observed increased communication between stromal and immune population including increased interactions between Type2 pneumocytes, endothelial cells, alveolar macrophages and lipid-laden macrophages (FIG. 65). Applicant observed elevated interaction potentials between endothelial and mast cells, including elevated IL-33 acting on mast cells through ST2 (IL1R1) (FIG. 66). Further, in high-burden lesions Applicant observed significant increases in receptor-ligand edge weights involving mast cell IL-13 signaling. Specifically, Applicant observed that IL-13 broadly acted on T cells and alveolar and lipid-laden macrophages (FIG. 67).

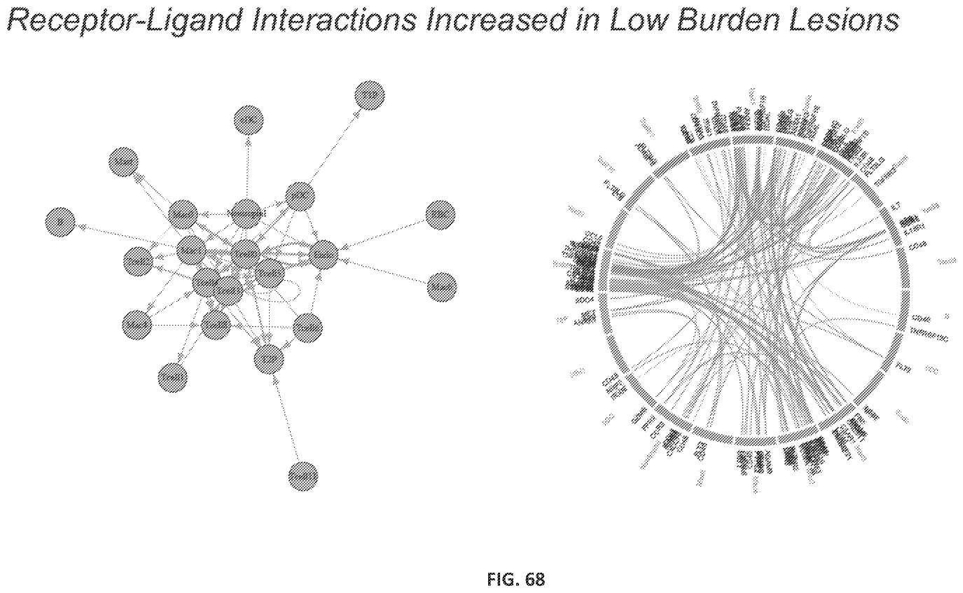

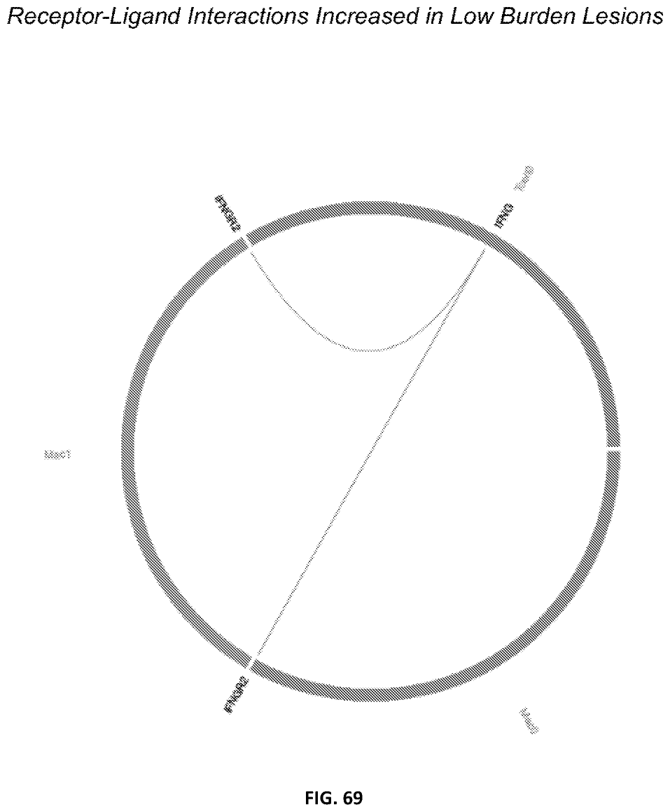

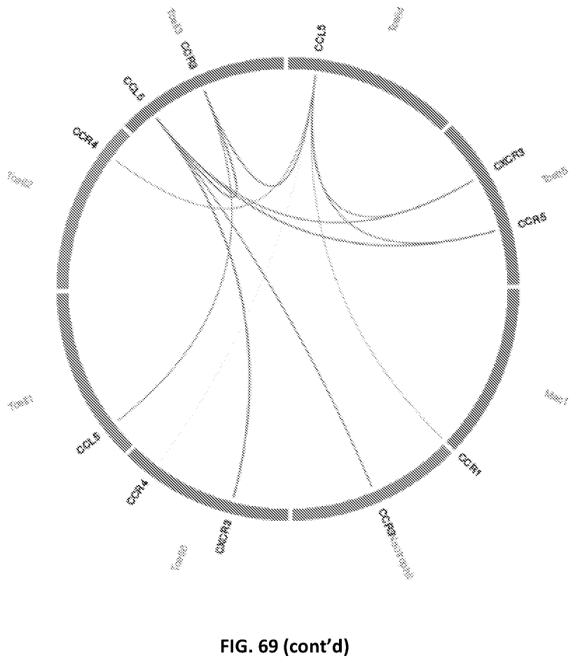

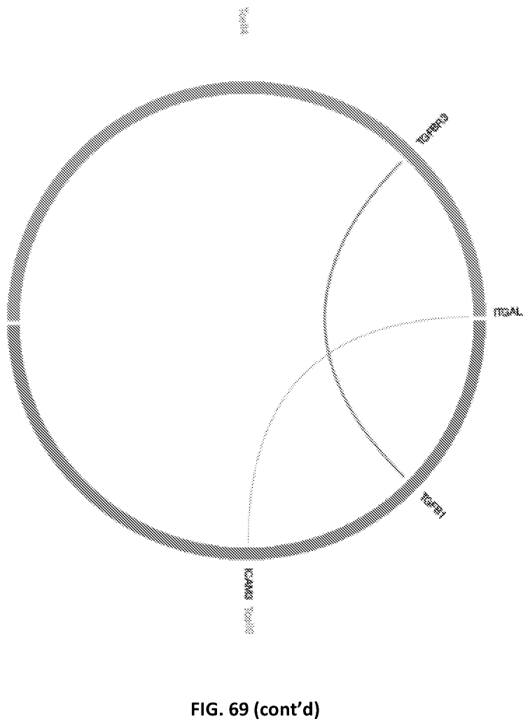

Receptor-Ligand Interactions in Low-Burden Lesions: Applicant generated a network map of cell-cell interactions generated from receptor-ligand edge weights that are significantly different between high and low burden lesions (over-represented in low burden). In low-burden lesions, Applicant observed significant increases in communication potentials involving Th1-Th17 cells and Effector CD8 T cells. Specifically, Applicant observed elevation of IFNγ signaling from Th1-Th17 cells acting on alveolar macrophages and monocytes via IFNγR2. Applicant further observe increased receptor-ligand edge weights involving CCL5 in low burden lesions. Applicant observed elevated crosstalk between Th1-Th17 cells and effector CD8 T cells via TGFβ1 and TGFβR3 signaling (FIG. 69). In low-burden lesions, Applicant observed significant increases in communication potentials involving Th1-Th17 cells and Effector CD8 T cells. Applicant observed increased network centrality between Th1-Th17 cells, Effector CD8 T cells, NK cells, proliferating T cells, and monocytes (FIG. 68).

Discussion

Applicant performed high-throughput single-cell profiling of 28 granulomas that spanned a wide range of bacterial burdens in a model that most closely recapitulated the features of human disease. Critically, by collecting lesions at 10 weeks post-infection, Applicant were able to profile lesions where differences in instantaneous bacterial burden were most likely to result from immune activity. This study design afforded a unique window into immune factors that mediate resistance to MTB infection at the level of individual granulomas. While Applicant observed broad similarity in the spectrum of immune cell states across high and low burden granulomas, the composition of immune populations varies significantly.

Th1-Th17 cells were described to result in protective immunity in the setting of both natural infection and vaccination. Emergence of a Th1-Th17 population in the lung played a central role in protective vaccination and natural immunity against numerous pathogens including B. pertussis, C. albicans, H. influenzae, P. aeruginosa, S. pneumoniae, Leishmaniasis, C. neoformans. Further, Th17 cells played an important role in M. tuberculosis infection and vaccine response.

Pathogenic Th17 Cells:

Studies of murine neuroinflammation (EAE) revealed the genetic basis of pathogenic Th17 differentiation and function. Notably, Th1-Th17 cells in MTB granulomas displayed elevated expression of many Th17 pathogenicity factors previously described in EAE including SATB126, FURIN27, BHLHE40, and RBPJ31. Specifically, BHLHE40 acted to drive a pathogenic transcriptional program in Th17 cells (CD93, IL-7R, CSF2, CCL3, CCL4, CCL5, TGFB3, ICOS, and LGALS3). SATB1 was observed to directly augment expression of genes that underlie pathogenic effector function in Th17 cells. Expression analysis between SATB1-competent and SATB1−/− Th17 cells revealed genes that govern the development of pathogenic and nonpathogenic Th17 cells (Bhlhe40, Csf2, Hif1a, Il17a, Il22, Il23r, Tbx21), many of which characterized Th1-Th17 cells in MTB granulomas. In pathogenic Th17 cells, RBPJ acted to directly activate expression of IL-23R while repressing the expression of IL-1031. Among granuloma Th1-Th17 cells, Applicant also observed elevated expression of HIF1-alpha, which has been shown to reinforce Th17 differentiation. In murine studies of pathogenic Th17-differentiation, much of the pathogenic effect has been shown to derive from expression of GM-CSF (CSF2); however, Applicant failed to observe significant expression of GM-CSF among Th1-Th17 cells in MTB granulomas.

Within the cluster of Th1-Th17 cells in MTB granulomas, Applicant observed both CD4+ and CD8+ T cells. Previous studies reported a class of CD8+ Th17 cells that arose in the lung following primary influenza infection.

Ex-Th17:

While the Th1-Th17 cells observed in MTB granulomas shared many features of classical Th17 cells, there was significant phenotypic plasticity of Th17 cells in vivo. Numerous studies have observed the generation of so-called ‘ex-Th17 cells’ or ‘Non-classical Th1 cells’, which represent a population of memory T cells that differentiate from Th17 cells. Fate-mapping experiments have shown the development of population of IFN-gamma producing cells that previously produced IL-17 prior to their conversion to an ex-Th17 phenotype via IL-2335. Consistent with the role of IL-23-IL-23R signaling in Ex-Th17 function, Applicant observed elevated expression of IL-23R expression among Th1-Th17 cells in MTB granulomas. Most recently, effector Th17 cells have been shown to give rise to a lung-resident memory population that is responsible for protection against Klebsiella infection. Importantly, this study performed expression-based characterization of Ex-Th17 cells which reveals key differences between Th17 and ex-Th17 cells including elevated expression of RBPJ and RORA. As previously observed, Ex-Th17 cells display reduced expression of IL-17A and RORC.

Following immunization in the lung, Th17 cells give rise to tissue-resident memory cells.

Model of Granuloma-Level Bacterial Control:

During the early stages of infection, dendritic cells migrate to draining lymph nodes, where they preferentially induce differentiation of Th1 and Th17 cells. The emergence of clonally expanded Th1-Th17 cells shapes the ability of newly formed granulomas to control bacteria by altering ecologic balance from immune tolerance to bacterial control. Granulomas that form at the earliest stages of infection do so in the absence of a robust adaptive immune response. Instead, tissue-level and innate immune responses are the predominate axis of response.

For example, coincident helminth infection ablate MTB immune control and correlate with reduced frequency of Th1-Th17 cells. In Schistoma mansoni infection, IL-13 is a critical dampener of the host immune response that enables granuloma formation and pathogen viability while ablating a pyrrhic host-immune response. In MTB granulomas, Applicant observed elevated expression of IL-13 in mast cells from high burden lesions. Moreover, Applicant observe higher mast-cell IL-13 receptor-ligand edge weights in high-burden lesions that influence numerous T cell and macrophage subsets. IL-13 was also observed to directly antagonize Th17 expression of IL-17. IL-13 deficiency augmented pathogen elimination and survival of mice infected with C. neoformans.

Collectively, the data suggest a model of granuloma development in which tissue-mediated cytokine signaling likely leads to early expansion of ST2+ immune populations (i.e. mast cells and tissue Tregs) that generated a tolerogenic immune environment, primarily directed at mitigation of tissue damage and maintenance of tissue function. Notably, Applicant observed proportional expansion of mast cells, elevated mast cell expression of IL-13, and increased IL-13 receptor-ligand edge weights in high burden granulomas.

The work provided a framework for understanding cellular and molecular correlates of bacterial control in MTB granulomas. Applicant identified changes in cell type composition and gene expression that associated with granuloma-level bacterial burden. This work provided evidence for the role of specific T cell populations in immunologic control of MTB infection, while simultaneously implicating immune and stromal populations in loss of bacterial control.

### REFERENCES

1. Gierahn, T. M. et al. Seq-Well: portable, low-cost RNA sequencing of single cells at high throughput. Nature Methods 14, 395-398 (2017).
2. Warfel, J. M. & Merkel, T. J. Bordetella pertussis infection induces a mucosal IL-17 response and long-lived Th17 and Th1 immune memory cells in nonhuman primates. Mucosal Immunology 6, 787-796 (2013).
3. Ross, P. J. et al. Relative Contribution of Th1 and Th17 Cells in Adaptive Immunity to Bordetella pertussis: Towards the Rational Design of an Improved Acellular Pertussis Vaccine. PLOS Pathogens 9, e1003264 (2013).
4. Fedele, G., Bianco, M., Debrie, A.-S., Locht, C. & Ausiello, C. M. Attenuated Bordetella pertussis Vaccine Candidate BPZE1 Promotes Human Dendritic Cell CCL21-Induced Migration and Drives a Th1/Th17 Response. The Journal of Immunology 186, 5388-5396 (2011).
5. Schnoeller, C. et al. Attenuated Bordetella pertussis Vaccine Protects against Respiratory Syncytial Virus Disease via an IL-17-Dependent Mechanism. Am J Respir Crit Care Med 189, 194-202 (2013).
6. Higgins, S. C., Jarnicki, A. G., Lavelle, E. C. & Mills, K. H. G. TLR4 Mediates Vaccine-Induced Protective Cellular Immunity to Bordetella pertussis: Role of IL-17-Producing T Cells. The Journal of Immunology 177, 7980-7989 (2006).
7. Pandiyan, P. et al. CD4+CD25+Foxp3+ Regulatory T Cells Promote Th17 Cells In Vitro and Enhance Host Resistance in Mouse Candida albicans Th17 Cell Infection Model. Immunity 34, 422-434 (2011).
8. Lin, L. et al. Th1-Th17 Cells Mediate Protective Adaptive Immunity against Staphylococcus aureus and Candida albicans Infection in Mice. PLOS Pathogens 5, e1000703 (2009).
9. Li, W. et al. Recognition of conserved antigens by Th17 cells provides broad protection against pulmonary Haemophilus influenzae infection. PNAS 115, E7149-E7157 (2018).
10. Wu, W. et al. Th17-stimulating Protein Vaccines Confer Protection against Pseudomonas aeruginosa Pneumonia. Am J Respir Crit Care Med 186, 420-427 (2012).
11. Liu, J. et al. Early production of IL-17 protects against acute pulmonary Pseudomonas aeruginosa infection in mice. FEMS Immunology & Medical Microbiology 61, 179-188 (2011).
12. Wright, A. K. A. et al. Experimental Human Pneumococcal Carriage Augments IL-17A-dependent T-cell Defence of the Lung. PLOS Pathogens 9, e1003274 (2013).
13. Moffitt, K. L. et al. TH17-Based Vaccine Design for Prevention of Streptococcus pneumoniae Colonization. Cell Host & Microbe 9, 158-165 (2011).
14. Moffitt, K. L., Malley, R. & Lu, Y.-J. Identification of Protective Pneumococcal TH17 Antigens from the Soluble Fraction of a Killed Whole Cell Vaccine. PLOS ONE 7, e43445 (2012).
15. Wu, W., Huang, L. & Mendez, S. A live Leishmania major vaccine containing CpG motifs induces the de novo generation of Th17 cells in C57BL/6 mice. European Journal of Immunology 40, 2517-2527 (2010).
16. Zhang, Y. et al. Robust Th1 and Th17 Immunity Supports Pulmonary Clearance but Cannot Prevent Systemic Dissemination of Highly Virulent Cryptococcus neoformans H99. The American Journal of Pathology 175, 2489-2500 (2009).
17. Müller, U. et al. IL-13 Induces Disease-Promoting Type 2 Cytokines, Alternatively Activated Macrophages and Allergic Inflammation during Pulmonary Infection of Mice with Cryptococcus neoformans. The Journal of Immunology 179, 5367-5377 (2007).
18. Murdock, B. J. et al. Early or Late IL-10 Blockade Enhances Th1 and Th17 Effector Responses and Promotes Fungal Clearance in Mice with Cryptococcal Lung Infection. The Journal of Immunology 193, 4107-4116 (2014).
19. Scriba, T. J. et al. Distinct, Specific IL-17- and IL-22-Producing CD4+ T Cell Subsets Contribute to the Human Anti-Mycobacterial Immune Response. The Journal of Immunology 180, 1962-1970 (2008).
20. Van Dis, E. et al. STING-Activating Adjuvants Elicit a Th17 Immune Response and Protect against Mycobacterium tuberculosis Infection. Cell Reports 23, 1435-1447 (2018).
21. Khader, S. A. et al. IL-23 and IL-17 in the establishment of protective pulmonary CD4+ T cell responses after vaccination and during Mycobacterium tuberculosis challenge. Nature Immunology 8, 369-377 (2007).
22. Gopal, R. et al. Unexpected Role for IL-17 in Protective Immunity against Hypervirulent Mycobacterium tuberculosis HN878 Infection. PLOS Pathogens 10, e1004099 (2014).
23. Khader, S. A. et al. IL-23 Compensates for the Absence of IL-12p70 and Is Essential for the IL-17 Response during Tuberculosis but Is Dispensable for Protection and Antigen-Specific IFN-γ Responses if IL-12p70 Is Available. The Journal of Immunology 175, 788-795 (2005).
24. Wozniak, T. M., Saunders, B. M., Ryan, A. A. & Britton, W. J. Mycobacterium bovis BCG-Specific Th17 Cells Confer Partial Protection against Mycobacterium tuberculosis Infection in the Absence of Gamma Interferon. Infection and Immunity 78, 4187-4194 (2010).
25. Costa, A. C. da et al. A New Recombinant BCG Vaccine Induces Specific Th17 and Th1 Effector Cells with Higher Protective Efficacy against Tuberculosis. PLOS ONE 9, e112848 (2014).
26. Yasuda, K. et al. Satb1 regulates the effector program of encephalitogenic tissue Th17 cells in chronic inflammation. Nat Commun 10, 1-14 (2019).
27. Oksanen, A. et al. Proprotein Convertase FURIN Constrains Th2 Differentiation and Is Critical for Host Resistance against Toxoplasma gondii. The Journal of Immunology 193, 5470-5479 (2014).
28. Lin, C.-C. et al. Bhlhe40 controls cytokine production by T cells and is essential for pathogenicity in autoimmune neuroinflammation. Nature Communications 5, (2014).
29. Lin, C.-C. et al. IL-1-induced Bhlhe40 identifies pathogenic T helper cells in a model of autoimmune neuroinflammation. Journal of Experimental Medicine 213, 251-271 (2016).
30. Yu, F. et al. The transcription factor Bhlhe40 is a switch of inflammatory versus antiinflammatory Th1 cell fate determination. Journal of Experimental Medicine 215, 1813-1821 (2018).
31. Horste, G. M. zu et al. RBPJ Controls Development of Pathogenic Th17 Cells by Regulating IL-23 Receptor Expression. Cell Reports 16, 392-404 (2016).
32. Shi, L. Z. et al. HIF1α-dependent glycolytic pathway orchestrates a metabolic checkpoint for the differentiation of TH17 and Treg cells. Journal of Experimental Medicine 208, 1367-1376 (2011).
33. Hamada, H. et al. Tc17, a Unique Subset of CD8 T Cells That Can Protect against Lethal Influenza Challenge. The Journal of Immunology 182, 3469-3481 (2009).
34. Yen, H.-R. et al. Tc17 CD8 T Cells: Functional Plasticity and Subset Diversity. The Journal of Immunology 183, 7161-7168 (2009).
35. Hirota, K. et al. Fate mapping of IL-17-producing T cells in inflammatory responses. Nature Immunology 12, 255-263 (2011).
36. Vesely, M. C. A. et al. Effector TH17 Cells Give Rise to Long-Lived TRM Cells that Are Essential for an Immediate Response against Bacterial Infection. Cell 178, 1176-1188.e15 (2019).
37. Christensen, D., Mortensen, R., Rosenkrands, I., Dietrich, J. & Andersen, P. Vaccine-induced Th17 cells are established as resident memory cells in the lung and promote local IgA responses. Mucosal Immunology 10, 260-270 (2017).
38. Modulation of Mycobacterial-Specific Th1 and Th17 Cells in Latent Tuberculosis by Coincident Hookworm Infection The Journal of Immunology. Available at: www.jimmunol.org/content/190/10/5161.short. (Accessed: 3rd Sep. 2019)
39. Mentink-Kane, M. M. et al. IL-13 receptor α2 down-modulates granulomatous inflammation and prolongs host survival in schistosomiasis. PNAS 101, 586-590 (2004).
40. Liu, Q. et al. IL-33-mediated IL-13 secretion by ST2+ Treg controls inflammation after lung injury. JCI Insight (2019). doi:10.1 172/jci.insight.123919

Materials and Methods

Ethics Statement

All experimental manipulations, protocols, and care of the animals were approved by the University of Pittsburgh School of Medicine Institutional Animal Care and Use Committee (IACUC). The IACUC adheres to national guidelines established in the Animal Welfare Act (7 U.S.C. Sections 2131-2159) and the Guide for the Care and Use of Laboratory Animals (8th Edition) as mandated by the U.S. Public Health Service Policy.

All macaques used in this study were housed at the University of Pittsburgh in rooms with autonomously controlled temperature, humidity, and lighting. Animals were singly housed in caging at least 2 square meters apart that allowed visual and tactile contact with neighboring conspecifics. The macaques were fed twice daily with biscuits formulated for nonhuman primates, supplemented at least 4 days/week with large pieces of fresh fruits or vegetables. Animals had access to water ad libitem. Because our macaques were singly housed due to the infectious nature of these studies, an enhanced enrichment plan was designed and overseen by our nonhuman primate enrichment specialist. This plan had three components. First, species-specific behaviors were encouraged. All animals have access to toys and other manipulata, some of which were filled with food treats (e.g. frozen fruit, peanut butter, etc.). These were rotated on a regular basis. Puzzle feeders foraging boards, and cardboard tubes containing small food items also were placed in the cage to stimulate foraging behaviors. Adjustable mirrors accessible to the animals stimulated interaction between animals. Second, routine interaction between humans and macaques were encouraged. These interactions occurred daily and consisted mainly of small food objects offered as enrichment and adhere to established safety protocols. Animal caretakers were encouraged to interact with the animals (by talking or with facial expressions) while performing tasks in the housing area. Routine procedures (e.g. feeding, cage cleaning, etc.) were done on a strict schedule to allow the animals to acclimate to a routine daily schedule. Third, all macaques were provided with a variety of visual and auditory stimulation. Housing areas contained either radios or TV/video equipment that played cartoons or other formats designed for children for at least 3 hours each day. The videos and radios were rotated between animal rooms so that the same enrichment was not played repetitively for the same group of animals.

All animals were checked at least twice daily to assess appetite, attitude, activity level, hydration status, etc. Following M. tuberculosis infection, the animals were monitored closely for evidence of disease (e.g., anorexia, weight loss, tachypnea, dyspnea, coughing). Physical exams, including weights, are performed on a regular basis. Animals were sedated prior to all veterinary procedures (e.g. blood draws, etc.) using ketamine or other approved drugs. Regular PET/CT imaging was conducted on most of our macaques following infection and had proved very useful for monitoring disease progression. The veterinary technicians monitored animals especially closely for any signs of pain or distress. If any were noted, appropriate supportive care (e.g. dietary supplementation, rehydration) and clinical treatments (analgesics) were given. Any animal considered to have advanced disease or intractable pain or distress from any cause was sedated with ketamine and then humanely euthanatized using sodium pentobarbital.

Research Animals

Four Cynomolgus macaques (Macaca fascicularis), >4 years of age, (Valley Biosystems, Sacramento, Calif.) were housed within a Biosafety Level 3 (BSL-3) primate facility as previously described and as above. Animals were infected with low dose M tuberculosis (Erdman strain) via bronchoscopic instillation of 7-12 colony-forming units (CFUs)/monkey to the lower lung lobe. Animals were infected for a period of 10 weeks and Infection was confirmed by tuberculin skin test conversion. Serial clinical, microbiologic, immunologic, and radiographic examinations were performed, as previously described.

Serial PET-CT Imaging

Animals underwent PET-CT scans after Mtb infection at 4 weeks, 8 weeks and pre necropsy (i.e. 10 weeks post-infection). Animals were sedated, intubated and imaged by 2-deoxy-2-18F-D-deoxyglucose (FDG) PET imaging (microPET Focus 220 preclinical PET scanner, Seimens Molecular Solutions) and CT scanner (Neurologica Corp) within our biosafety level 3 facility as previously described. The total lung FDG avidity was analyzed using Osirix viewer, an open-source PACS workstation and DICOM viewer. The whole lung was segmented on CT by using the Growing region algorithm on the Osirix viewer to create a ROI of normal lung (Hounsfield units <200). The closing tool was used to include individual nodules and other pulmonary disease. The ROI was transferred to the co-registered PET scan and manually edited to ensure all pulmonary disease was included. Voxels outside the ROI were set to zero and voxels with an SUV greater than or equal to normal lung (SUV >2.3) were isolated. Finally, the “Expert ROIs” plug-in was then used to export the data from these isolated ROIs to a spreadsheet where the total SUV per voxel were summed to represent the total lung FDG avidity.

Necropsy

Necropsy was performed as previously describe. Briefly, an 18F-FDG PET-CT scan was performed on every animal 1-3 days prior to necropsy to measure disease progression and identify individual granulomas as described. At necropsy, monkeys were maximally bled and humanely sacrificed using pentobarbital and phenytoin (Beuthanasia; Schering-Plough, Kenilworth, N.J.). Individual lesions previously identified by PET-CT and those that were not seen on imaging from lung and mediastinal lymph nodes were obtained for histological analysis, bacterial burden, and immunological studies. A veterinary pathologist described gross pathologic findings. To quantify gross pathologic disease (disease burden), a necropsy score was developed in which points were given for TB disease: number, size, and pattern of granulomas distributed in each lung lobe and mediastinal lymph node and in other organs each lung lobe, lymph node, and visceral organ were included and enumerated, and an overall score was determined as previously described. The size of each granuloma was measured at necropsy and by pre necropsy scan. Representative sections of each tissue were homogenized into single-cell suspensions for immunologic studies, flow cytometric analysis, and bacterial burden, as previously described.

Bacterial Burden

200 ul of each granuloma homogenate were plated in serial dilutions onto 7H11 medium, and the CFU of M. tuberculosis growth were enumerated 21 days later to determine the number of bacilli in each granuloma. As a quantitative measure of overall bacterial burden, a CFU score was derived from the summation of the log-transformed CFU/gram of each sample at the time of necropsy, as previously described.

Sequencing of Barcoded Bacteria Libraries

Lineage relationships were determined between granulomas through the use of bacterial barcodes. DNA was extracted from whole granuloma lysates and sequencing was performed to assign barcodes to individual granulomas. Relationships between granulomas (i.e. parent-daughter lesions) were determined through a combination of bacterial barcode, granuloma age (PET-CT), and spatial location.

Single-Cell mRNA Sequencing

Applicant performed high-throughput single-cell mRNA sequencing using the Seq-Well platform (Gierahn et al. Nature Methods 2017). Initially, a single-cell suspension was obtained from granulomas isolated at necropsy. Intact granulomas were isolated via manual dissection and carefully separated from surrounding parenchyma. Isolated granulomas were cut into smaller pieces using a razor blade, before being digested with DNAse and Collagenase I and mechanically dissociated using a GentleMac. For each granuloma, a single-cell suspension was obtained and applied to the surface of a loaded Seq-Well device. Following cell loading, Seq-Well devices were reversibly sealed with a polycarbonate membrane and incubated at 37 C for 30 minutes. After membrane sealing, Seq-Well devices were submerged in lysis buffer (5M guanidine thiocyanate, 10 mM EDTA, Beta-mercaptoethanol, Sarkosyl) and rocked for 20 minutes. Following cell lysis, arrays were rocked for 40 minutes in 2M NaCl to promote hybridization of mRNA to bead-bound capture oligos.

Reverse Transcription and PCR Amplification

Beads were removed from arrays by centrifugation and reverse transcription was performed at 52 C for 2 hours. Following reverse transcription arrays were washed with TE-SDS (TE Buffer+0.1% SDS) and twice with TE-Tween (TE Buffer+0.01% Tween20). Following ExoI digestion, PCR amplification was performed to generate whole-transcriptome amplification (WTA) libraries. Specifically, a total of 2,000 beads were amplified in each PCR reaction using 16 cycles as previously described 10. Following PCR amplification, SPRI purification was performed at 0.6× and 0.8× volumetric ratios and eluted samples were quantified using a Qubit. Sequencing libraries were prepared by tagmenting 800 pg of cDNA input using Illumina Nextera XT reagents. Tagmented libraries were purified using 0.6× and 0.8× volumetric SPRI ratios and final library concentrations were determined using a Qubit. Library size distributions were established using an Agilent TapeStation with D1000 High Sensitivity ScreenTapes (Agilent, Inc., USA).

Sequencing and Alignment

Libraries for each sample were sequenced on a NextSeq550 75 Cycle High Output sequencing kit (Illumina Inc., Sunnyvale, Calif., USA). For each library, 20 bases were sequenced in read 1, which contains information for cell barcode (12 bp) and unique molecular identifier (UMI, 8 bp), while 50 bases were obtained for each read 2 sequence. Cell barcode and UMI tagging of transcript reads was performed using DropSeqTools (Macosko et al., 2015). Barcode and UMI-tagged sequencing reads were aligned to the Macaca fascicularis v5 genome (https://useast.ensembl.org/Macaca_fascicularis/Info/Index) using the STAR aligner. Aligned reads were then collapsed by barcode and UMI sequences to generate digital gene expression matrices for each array.

Data Processing and Quality Control

Initially, Applicant generated a combined dataset of >202,000 cells after applying thresholds of 500 genes and 750 transcripts (UMIs). Applicant initially visualized cells from each array using t-SNE and performed clustering in Seurat. For many arrays, Applicant initially observed large clusters of cell barcodes that pass inclusion thresholds (i.e. >500 genes and >750 transcripts) but were marked by distinct features of cell-type defining gene expression. Instead, these cells were marked largely by genes presumed to originate from other cell types (e.g. HBB from erythrocytes, JCHAIN from plasma cells, and CPA3 from mast cells). Applicant performed UMAP dimensionality reduction and Louvain clustering (Resolution=4.25) across 202,000 barcodes, including the cell barcodes of undetermined identities. To focus the analysis conserved cell populations, Applicant excluded clusters that were derived primarily from a single lesion (40% or greater from a single lesion). In total, Applicant removed barcodes presumed to originate from a combination of extracellular mRNA and cellular debris inherent to the necrotic microenvironment of granulomas.

Correction for Background Contamination

In initial analysis, Applicant observed a significant amount of background contamination marked by ectopic expression of cell-type defining genes (e.g. widespread expression of JCHAIN, HBB, and CPA3 etc.). Specifically, Applicant observed this contamination to vary in relation to the overall distribution of cell-types recovered from each array. For each array, Applicant used SoupX 19 to (1) generate array-specific profiles of background contamination and (2) estimate per-cell contamination fractions, and (3) generate corrected background-corrected UMI counts matrices. To generate background expression profiles, Applicant first generated counts matrices containing up to 50,000 barcodes to assemble a collection of low-UMI cell barcodes that presumably represent extracellular mRNA. For each array, a UMI threshold for background expression was determined using EmptyDrops 20 to estimate the likelihood distribution of low-UMI barcodes have optimal cellular identity. Using an array-specific UMI-threshold (Range: 20-100 UMIs, Table SX), Applicant separately generated composite background profiles for each array. To estimate per-cell contamination fraction, Applicant first identified a set of bimodally expressed genes, which were subsequently used to estimate contamination fraction in cells for which endogenous gene expression was not assumed. To obtain per-cell estimates of the contamination fraction, the estimated contamination fraction was interpolated across the UMI distribution. Finally, the composite soup profile was subtracted from each cell's transcriptional profile using a correction factor based on the estimated contamination fraction. For each array, Applicant removed individual transcripts most likely to be contamination from each single-cell based on the estimated contamination fraction. Specifically, individual transcripts were sequentially removed from each single-cell transcriptome until the probability of subsequent transcripts being soup-derived was less than 0.5 to generate a background-corrected counts matrix for each array.

Separation of Doublets

Applicant performed doublet identification and separation using DoubletFinder. To account for differences in cell loading densities and expected cell doublets, Applicant generated array specific estimates of the expected number of doublets. For example, a total of 20,000 cells applied to a Seq-Well device containing 85,000 wells (lambda=20,000), Applicant would calculate an expected doublet rate of 2.37%. For each array, Applicant generated pseudo-doublets using the following parameter values in DoubletFinder: proportion.artificial=0.25 and proportion.NN=0.01. Cells were identified as doublets based on their rank order in the distribution of the proportion of artificial nearest neighbors (pANN). Specifically, Applicant identified the pANN value for the cell at the expected doublet percentile and used the corresponding pANN value as a threshold to remove additional cells with pANN greater than or equal to this value.

Cell Type Identification

Initially, cell barcodes with greater than 750 transcripts and 500 detected genes were included in subsequent analyses. For each granuloma, variable genes were identified as those genes with expression-variance ratios above a threshold and principal components analysis was performed using variable genes identified within each granuloma individually. Removal of low-quality cells was initially performed at the level of individual granulomas. Within each granuloma, cell clusters defined only by mitochondrial gene expression[AKS3] were removed from subsequent analysis. Cells from individual granulomas were combined by animal and integrated analysis was then performed at the level of each animal. Across all cells obtained from each monkey, Applicant performed variable gene identification[AKS4], principal components analysis, t-SNE dimensionality reduction, and cluster identification. Finally, cells were combined across all animals to generate a combined dataframe containing 113, cells. For the combined dataset, variable gene identification, principal components analysis, t-SNE and clustering were performed using Scanpy.

Cell Type Classification

At this point, consistency of cell type classification was used as a metric to identify and remove doublets and low-quality cells. Specifically, cell type classifications were made at the level of generic cells (e.g. ‘T cells’, ‘Macrophages’, ‘Endothelial Cells’, etc.) in datasets containing all cells from each animal and all cell combined. Only those cell consistently identified as the same generic cell type (e.g. ‘T cells’, ‘Macrophages’, ‘Mast cells’) at the level of animal and across all granulomas were retained in the final ‘cleaned’ dataset. Downstream analysis was performed using the final dataset of 113,864 cells with conserved.

Cell Type Assignment of Proliferating Cells

Applicant identified a cluster of cells primarily defined by markers of cellular proliferation (MKI67, TOP2A, and CDK1) (Figure SX). To understand the underlying cell type identity of these cells, Applicant performed separate dimensionality reduction and clustering. Specifically, Applicant performed UMAP dimensionality reduction and Louvain clustering (resolution=0.8) and identified a total of 8 clusters (Table SX). Applicant performed enrichment analysis across identified clusters and assigned cell-type identities based on similarity to other cell-type signatures. Through this approach, Applicant identified distinct clusters of proliferating macrophages, T cells, neutrophils, and mast cells that were re-assigned to their respective cell type clusters.

Analysis of T Cell Sub-Types

To identify variation in the composition of T cell and macrophage populations, Applicant performed sub-analysis across all T cells and Macrophages. To avoid artifactual associations in dimensionality reduction or sub-type clustering, Applicant removed the set of soup defining genes (per array soup scores >0.001) across arrays that defined the unit of analysis. To avoid removal of biologically informative genes (i.e. soup-defining genes derived from T cells or macrophages), Applicant excluded the top 50 cell-type defining genes for T cells or Macrophages. Across the set of all T cells and within each animal, Applicant performed dimensionality reduction to generate force-directed graphs (citation) and Louvain clustering to identify sub-types.

Single-Cell Analysis of Normal Lung

Applicant performed scRNA-seq on cells obtained from normal lung from a single non-human primate. In total, Applicant ran Seq-well arrays from multiple regions of the lung. For each array from normal lung, Applicant removed low-quality cells on the basis of the lack of distinguishing features and performed array-specific background correction using SoupX and removed doublets using DoubletFinde. After robust quality filtering, Applicant performed dimensionality reduction and Louvain clustering across the cells from normal lung. Applicant performed enrichment analysis to obtain expression signatures for each cell type cluster. Cell-types were assigned based on a combination of literature-based curation and comparison to published bulk and single-cell signatures of airway epithelium.

Comparison of T Cell Signatures

Applicant performed extensive comparisons of literature-based gene expression signatures to deeply characterize the cellular and functional identity of T cell clusters observed in MTB granulomas. Briefly, Applicant generated cell signature scores in Seurat using the AddModuleScore function using gene expression signatures across T cell clusters obtained from human lung cancer. Applicant performed similar comparisons for numerous other single-cell mRNA sequencing studies that include large-scale characterization of T cell phenotype. In each case, Applicant performed 1,000 permutations reordering both module scores and T cell cluster identities to establish significance of similarity to literature-based signatures from single-cell data.

Applicant performed comparisons to a range of gene expression signatures derived from population mRNA sequencing in the Savant database 13. First, Applicant generated population averages across all cells within each T cell sub-cluster across all genes. This aggregated data across T cell subsets was then uploaded to the Savant database. Applicant further performed gene-set enrichment analysis using gene lists obtained from the Savant database for each of the signatures. Specifically, gene-set enrichment was performed in Piano using a hypergeometric test with correction for multiple hypothesis testing using the Benjamini-Hochberg method for false discovery.

Granuloma-Level Correlates of Bacterial Burden, Granuloma Size and PET Intensity

Applicant examined cellular and molecular correlates of granuloma-level bacterial burden in a number of ways. Initially, Applicant calculated the proportion of cell type clusters observed in each granuloma individually. Applicant performed correlation analysis of cell-type proportions and end-point CFU level across all granulomas using a Spearman-rank correlation test. All spearman rank correlation tests were adjusted for animal-specific effects and corrected for multiple testing using Benjamini-Hochberg False-Discovery correction. Applicant used to time-resolved PET-CT data to classify granulomas according to their temporal dynamics in both size and PET intensity. To effectively capture the temporal dynamics of PET-CT Applicant scaled PET intensity and size data by the maximum observed value for each granuloma. Applicant then performed k-means clustering using the scaled values to identify lesions with similar temporal dynamics. Initially, Applicant examined the relationship between trajectory classifications and bacterial burden (CFU) and cell-type composition.

Association Between Cell Type Composition and Granuloma-Level Metadata

Applicant performed associations between cell type composition and granuloma-level metadata at a number of levels. For comparisons of generic cell types across the combined set of 28 granulomas, Applicant performed Spearman rank correlation tests was used to assess the relationship between bacterial burden and cell-type abundance for each cell-type independently. For direct comparisons of high and low-burden lesions, Applicant performed Student's T-test to compare the proportional cell type composition between groups. Student's T-test was used to determine the relationship between proportional cell-type composition and other dichotomous variables (e.g. timing of granuloma formation).

T Cell Functional Analysis

To identify functional gene expression signatures of bacterial control, Applicant performed differential expression analysis within each T cell subset using a Wilcox test. For each gene, comparisons were made between T cells recovered from high and low burden granulomas and correction for multiple testing was performed (Benjamini-Hochberg). Applicant further performed directed analysis of T cell exhaustion and cytotoxic effector function. In each case, Applicant examined the expression of individual genes in addition to combined metrics of gene expression across multiple genes. In the latter case, Applicant used the AddModuleScore function in Seurat to generate signature

Single-Cell TCR Reconstruction

Applicant performed single-cell TCR reconstruction across all 28 granulomas. Initially, Applicant performed targeted enrichment of TCR constant genes using biotinylated probes (Cite Tu et al.). DNA was denatured with a 15 minute incubation at 95 C, 80-mer hybridization probes (5′ biotin) specific to alpha and beta TCR constant chain were added along with a blocking oligo (SMART universal PCR primer) to prevent re-annealing of denatured cDNA molecules. Hybridization was accomplished through a 60 minute incubation at 65 C. Hybridized products were enriched using streptavidin coated magnetic beads (M-270 Dynabeads). Magnetic beads were sequentially washed with buffers contained in the IDT hybridization kit (e.g. Wash Buffer 1, Stringent Wash Buffer, Wash Buffer 2, and Wash Buffer 3; Cat #IDT DNA, Inc.). After washing beads, PCR was performed to amplify enriched cDNA molecules. Amplified TCR products were purified using custom SeraPure Beads at a 0.64× ratio. Following an initial primer extension, additional PCR amplification was performed using a series of V-specific primer sequences.

Co-Variation in Granuloma Composition

Applicant calculated correlations in cell-type proportions to identify underlying structure in the co-occurrence of cell types across all granulomas. Specifically, pearson correlations were calculated for all pair-wise cell-type comparisons and structure of correlations across cell types was examined using hierarchical clustering.

Cell-Communication Analysis

To examine cell-cell interactions, Applicant first generated a curated list of receptor-ligand pairs through a combination of publicly-available databases and literature review. Within each granuloma, Applicant generated edge weights between cell types for a given receptor ligand pair by multiplying the average receptor expression in Cell Type 1 by the average ligand expression in Cell Type 2. Edge weights were constructed for all receptor-ligand pairs and pairwise-cell type combinations within granulomas individually. Within each granuloma, Applicant performed a total of 1000 permutations for each receptor-ligand pair in which cell-type identifiers were randomly resorted and the resulting edge weight was recorded. For each receptor-ligand pair, the significance of the observed value was calculated from a z-score comparison of the observed value relative the permuted values.

Applicant further performed adjustment of receptor-ligand edge weights at multiple levels. (1) Applicant examined differences in unweighted receptor ligand edge weights Comparisons of receptor-ligand edge weights on the basis of bacterial burden (granuloma-level CFU) and timing of granuloma formation (time of first PET-CT observation). (2) To account for differences in the relative abundance of ‘sender’ cell types, Applicant multiplied receptor-ligand edge weights by the proportion of ‘sender’ cell types. In effect, this generates a pool of ‘sender’ cell derived ligand that is available to act upon cell types bearing appropriate receptors. (3) To account for variability in the expression of receptor expression across cell population, Applicant weighted receptor-ligand edge-weights by the proportion of total receptor expression within the receiving cell type cluster relative the aggregate receptor expression. In this scheme, receptors with more uniform expression across cell type clusters will be down-weighted to reflect non-autonomous sinks of extracellular ligands, while receptors predominantly expressed by a single cell type will be up-weighted. (4) Finally, Applicant adjusted receptor-ligand edge weights to account for the percent of cells within the receiver population expressing a given receptor.

To identify axes of intercellular communication with differential weights across granulomas, Applicant performed t-tests of receptor-ligand edge weights between (1) high-burden and low-burden lesions, (2) original and late-blooming lesions. Applicant filtered results based on the following criteria: (1) average permutation p-values within high or low-burden lesions <0.05, (2) p-value from student's t-test <0.05, (3) fold-change >0, and (4) receptor-ligand edge weights were ranked by receptor-ligand edge weights.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method for treatment or prophylaxis of a Mycobacterium tuberculosis (MTB) infection comprising modulating expression of one or more genes in a mast cell, a plasmablast, or a combination thereof, in a subject in need thereof, wherein the one or more genes expresses at a level different in a resolving granuloma in an MTB infected tissue comparing to a level in a progressing granuloma in the MTB infected tissue.

2. The method of claim 1, wherein the modulating comprises upregulating the expression of the one or more genes, wherein the one or more genes expresses at a higher level in a resolving granuloma in an MTB infected tissue comparing to a level in a progressing granuloma in the MTB infected tissue.

3. The method of claim 1, wherein the modulating comprises downregulating the expression of the one or more genes, wherein the one or more genes expresses at a lower level in a resolving granuloma in an MTB infected tissue comparing to a level in a progressing granuloma in the MTB infected tissue.

4. The method of claim 1, wherein the modulating comprises delivering one or more agonists of the one or more genes to a subject.

5. An engineered mast cell or plasmablast comprising elevated expression of one or more genes that expresses at a level higher in a resolving granuloma in an MTB infected tissue comparing to a level in a progressing granuloma in the MTB infected tissue.

6. An engineered mast cell or plasmablast comprising reduced expression of one or more genes that expresses at a level lower in a resolving granuloma in an MTB infected tissue comparing to a level in a progressing granuloma in the MTB infected tissue.

7. A method of identifying a population of cells correlating to a granuloma characteristic, the method comprising:
a. obtaining a first plurality of cells from one or more granuloma with the characteristic and a second plurality of cells from one or more granuloma without the characteristic;
b. sequencing nucleic acid molecules in the first and the second pluralities of cells using single cell sequencing;
c. clustering genes differently expressed between the first and the second plurality of cells; and
d. identifying the population of cells based on the clustering of the different expressed genes.

8. The method of claim 7, further comprising excluding a cluster of genes expressing only in a single granuloma.

9. The method of claim 7, further comprising identifying the population of cells in different subjects.

10. The method of claim 7, wherein the granuloma characteristic is progressiveness.

11. A method of determining a characteristic of a granuloma in a subject infected by MTB, the method comprising identifying the population of cells according to claim 7.

12. A method of treatment or prophylaxis of a Mycobacterium tuberculosis (MTB) infection comprising activating a p53 pathway in macrophages of or from a patient in need thereof.

13. A method of treatment or prophylaxis of a Mycobacterium tuberculosis (MTB) infection comprising delivering a p53 agonist to a patient in need thereof.

14. A method of treatment or prophylaxis of a Mycobacterium tuberculosis (MTB) infection comprising delivering a p53 agonist to a patient's macrophages.

15. The method of claim 13 or 14, wherein the p53 agonist is a p53 pathway agonist.

16. The method of claim 15, wherein the p53 agonist is delivered to the patient's macrophages in vivo.

17. The method of claim 12 or 13, wherein the macrophages are activated or agonized in vivo.

18. The method of claim 12 or 13, wherein the macrophages are activated or agonized in a sample from the patient or ex vivo, and optionally, subsequently returned to the patient.

19. The method of claim 15, wherein the p53 agonist is delivered to the patient's macrophages in a sample from the patient or ex vivo, and optionally, subsequently returned to the patient.

20. The method of claim 12 or 13, wherein the p53 pathway activator, p53 agonist, or p53 pathway agonist is an MDM2 inhibitor.

21. The method of claim 20, wherein the MDM2 inhibitor is nutilin-3a.

22. The method of claim 12 or 13, wherein control of MTB infection by macrophages in the patient is promoted.

23. The method of claim 12 or 13, wherein the macrophage is, or is derived from, a primary human CD14+ monocyte-derived macrophage (MDM).

24. The method of claim 12 or 13, wherein at least one of the following genes are upregulated: TOP2B, SORT1, NUDT3, IRF4, CXCL1.

25. A method of differentiating one or more macrophage subpopulations infected by MTB from one or more uninfected macrophage subpopulations, the method comprising:
a. assaying the macrophages for the presence, or overexpression compared to wt macrophages, of:
i. at least one of cytokine receptors (including IFNGR1, IL1RN), SLAM family members (including SLAM7, SLAMF5), and kinases (including HCK, CAMK1), or
ii. at least one of differentiators of macrophage state, including M1 and M2, HLA-DRB1, and CD68, in particular CD86;
b. assaying the macrophages for the presence, or overexpression compared to wt macrophages, of at least one of:
i. at least one of ApoE, CD36, CD52, and IL-8 ApoE, CD36, CD52, IL8
c. identifying the one or more infected macrophage subpopulations based on the assay in a);
d. identifying the one or more uninfected macrophage subpopulations based on the assay in b); and optionally,
e. separating the one or more infected macrophage subpopulations from the one or more uninfected macrophage subpopulations based on the identifications made in c) and d); wherein separating optionally comprises
i. by labelling or tagging one of the infected or the uninfected subpopulations; or
ii. by differentially labelling or tagging the infected and the uninfected subpopulations.

26. The method of claim 25, wherein identified, and optionally separated, infected macrophage subpopulations are contacted with a p53 agonist or p53 pathway agonist to promote a control phenotype.

27. The method of treatment of a Mycobacterium tuberculosis (MTB) infection of any one of claims 12-25, comprising activating the p53 pathway in macrophages of or from the patient to promote a control phenotype.

28. A method prophylaxis of a Mycobacterium tuberculosis (MTB) infection, comprising activating a p53 pathway in macrophages of or from a patient exposed to or at risk of MTB infection, optionally to promote a control phenotype.

29. A method of treatment or prophylaxis of an Mycobacterium tuberculosis (MTB) infection comprising activating a NF-κB pathway in macrophages of or from a patient in need thereof.

30. A method of treatment or prophylaxis of a Mycobacterium tuberculosis (MTB) infection comprising activating a Vitamin D Receptor (VDR) pathway in macrophages of or from a patient in need thereof.

31. A CD14+ macrophage model cell or cell line, wherein: at least one of the following genes are upregulated: CD206, CD86, CD32; and/or at least one of the following genes are downregulated: CD163.

32. The model cell or cell line of claim 12, which is or is derived from a primary human CD14+ monocyte-derived macrophage (MDM).

33. A method of treating or preventing a disease by modulating a microenvironment of a cell or cell mass in a subject, the method comprising administrating an effective amount of one or more modulating agents that modulate mast cells, plasma cells, Th1-Th17 cells, and/or CD8+ T cells in the subject.

34. The method of claim 33, wherein the modulating agent reduce number or function of mast cells.

35. The method of claim 33, wherein the one or more modulating agents modulates expression of one or more genes in mast cells.

36. The method of claim 35, wherein the one or more genes in mast cells comprises genes in IL-13 signaling pathway and/or genes in IL-33 signaling pathway.

37. The method of claim 35, wherein the one or more genes in mast cells comprises IL-33, IL-1R1, and/or IL-13.

38. The method of claim 33, wherein the one or more modulating agents increase number or function of Th1-Th17 cells.

39. The method of claim 33, wherein the one or more genes in Th1-Th17 cells.

40. The method of claim 39, wherein the one or more genes in Th1-Th17 cells comprises genes in INF-γ signaling pathway and/or genes in TGFβ signaling pathway.

41. The method of claim 39, wherein the one or more genes in Th1-Th17 cells comprises INF-γ, INF-γ receptor 2, TGFβ1, TGFβ receptor 3, CCL5, and/or IL-23R.

42. The method of claim 39, wherein the one or more genes in Th1-Th17 cells comprises IL-2RG, IFN-γ, IFI27, LAG3, TIGIT, CD8A, NKG7, CCL20, CCL3, and/or CCL5.

43. The method of claim 33, wherein the one or more modulating agents modulates expression of:
a. GZMA, GZMB, GNLY, and PRF1,
b. CCR7, LEF1, and SELL in Naïve T cells,
c. FOXP3, IKZF2, and IL1RL1 in regulatory T cells,
d. OAS2, MX1, and ISG15 in interferon-responsive cells,
e. GZMK, CCL5, and CXCR4 in CD8+ T cells,
f. CX3CR1, GZMB, and ZEB2 in CD8+ T cells,
g. MKI67 and TOP2A in proliferating T cells,
h. APOC1, APOE, and C1QB in alveolar macrophages,
i. TIMP1 and IDO1 in monocytes,
j. LIPA and MAN2B1 in macrophages,
k. MRC1, FABP5, and PPARG in lipid-laden macrophages,
l. CP, CXCL9, and NFKB1 in inflammatory macrophages,
m. MKI67 and TOP2A in proliferating macrophages,
n. BIRC3, CCR7, and LAMP3 in myeloid dendritic cells,
o. BHLHE40, SATB1 and RBPJ in Th17 cells,
p. IFNG, CCL4, RORC, IL17A, IL17F, IL1R1, RORA, IRF4, and RBPJ in Th17 cells,
q. IL23R, IL7R, NDFIP1, ILI1R1, RORA, IRF4, and RBPJ in Ex-Th17 cells,
r. KLF2, TGFBR3, CX3CR1, and GZMB in CD8+ T cells,
s. FOXP3, TIGIT, GITR, and GATA3 in ST2+ regulatory T cells,
t. IL2RG, IFNG, IFI27, LAG3, TIGIT, CD8A, NKG7, CCL20, CCL3, and CCL5 in Th1-Th17 cell, or
u. any combination thereof.

44. The method of claim 33, wherein the one or more modulating agents is comprised in a vaccine formulation.

45. The method of claim 33, wherein the disease is bacterial infection, tuberculosis, cancer, chronic rhinosinusitis, asthma, allergy, wound, or a combination thereof.

46. The method of claim 33, wherein the disease is a latent disease.

47. The method of claim 33, wherein the disease is an active disease.

48. The method of claim 33, wherein the cell or cell mass is a granuloma.

49. The method of claim 33, wherein the one or more modulating agents comprises an antibody, or antigen binding fragment, an aptamer, affimer, non-immunoglobulin scaffold, small molecule, genetic modifying agent, or a combination thereof.

50. A method of treating a disease in a subject comprising:
a. contacting one or more mast cells and/or Th1-Th17 cells with one or more modulating agents, wherein the one or more modulating agents activates
i. IL-33, IL-1R1, and genes in IL-13 signaling pathway in the mast cells, and/or
ii. INF-γ, INF-γ receptor 2, TGFβ1, TGFβ receptor 3, CCL5, and genes in INF-γ and TGFβ signaling pathway in the Th1-Th17 cells;
b. administering the mast cells and/or Th1-Th17 from a) to the subject.

51. The method of claim 50, wherein the mast cells and/or Th1-Th17 cells are isolated or derived from the subject.

52. A mast cell or cell line expressing one or more of: IL-33, IL-1R1, and genes in IL-13 signaling pathway.

53. A Th1-Th17 cell expressing one or more of: INF-γ, INF-γ receptor 2, TGFβ1, TGFβ receptor 3, CCL5, and genes in INF-γ and TGFβ signaling pathway.

54. A vaccine comprising the one or more modulating agents in any one of claims 33 to 49.
